(19) United States
(12) Patent Application Publication
(10) Pub. No.: US 2025/0011307 A1
(43) Pub. Date: Jan. 9, 2025

(54) CRYSTALLINE FORMS OF BIARYL YAP/TAZ-TEAD PROTEIN-PROTEIN INTERACTION INHIBITORS

(71) Applicant: Novartis AG, Basel (CH)

(72) Inventors: Runyan LI, Shanghai (CN); Bo LIU, Shanghai (CN); Michael MUTZ, Freiburg im Breisgau (DE); Lina YUAN, Shanghai (CN); Yingcong ZHOU, Shanghai (CN)

(21) Appl. No.: 18/687,609

(22) PCT Filed: Aug. 30, 2022
(86) PCT No.: PCT/IB2022/058131

§ 371 Date: Sep. 24, 2024

(30) Foreign Application Priority Data
Sep. 1, 2021 (WO) PCT/CN2021/115963

(51) Int. Cl. C07D 405/04; A61K 31/343; A61K 31/4025; C07D 307/81

(52) U.S. Cl. CPC C07D 405/04; A61K 31/343; A61K 31/4025; C07D 307/81

(57) ABSTRACT

The present invention generally relates to crystalline polymorphic forms of the biaryl YAP/TAZ-TEAD protein-protein interaction inhibitors 4-((2S,4S)-5-Chloro-6-fluoro-2-phenyl-2-((S)-pyrrolidin-2-yl)-2,3-dihydrobenzofuran-4-yl)-5-fluoro-6-(2-hydroxyethoxy)-N-methylnicotinamide and 2-((2S,3S,4S)-5-Chloro-6-fluoro-3-methyl-2-((methylamino)methyl)-2-phenyl-2,3-dihydrobenzofuran-4-yl)-3-fluoro-4-methoxybenzamide and salts thereof, as well as methods of using the forms in the treatment of cancer.

### FIELD OF THE DISCLOSURE

The present invention generally relates to crystalline polymorphic forms of the biaryl YAP/TAZ-TEAD protein-protein interaction inhibitors 4-((2S,4S)-5-Chloro-6-fluoro-2-phenyl-2-((S)-pyrrolidin-2-yl)-2,3-dihydrobenzofuran-4-yl)-5-fluoro-6-(2-hydroxyethoxy)-N-methylnicotinamide and 2-((2S,3S,4S)-5-Chloro-6-fluoro-3-methyl-2-((methylamino)methyl)-2-phenyl-2,3-dihydrobenzofuran-4-yl)-3-fluoro-4-methoxybenzamide and salts thereof, as well as methods of using the forms in the treatment of cancer.

### BACKGROUND

Normal tissue growth, as well as tissue repair and remodeling, require specific control and regulated balance of transcriptional activity. Transcriptional output is coordinated through a number of key signaling modules, one of which is the Hippo pathway. Genetic studies in Drosophila and mammals have defined a conserved core signaling cassette, composed of Mst1/2 and Lats1/2 kinases which inhibit the transcriptional co-activators YAP and TAZ (official gene name: WWTR1).

An activated Hippo pathway translates to YAP and TAZ being phosphorylated and sequestered/degraded in the cytoplasm. Upon inactivation of the Hippo pathway, YAP and TAZ translocate to the nucleus and associate with transcription factors, namely members of the TEAD family (TEAD1-4). The YAP/TAZ-TEAD complexes in turn promote transcription of downstream genes involved in cellular proliferation, death and differentiation. While YAP and TAZ can also interact with a number of other factors, TEADs are commonly accepted to be the key mediators of the growth-promoting and tumorigenic potential of YAP and TAZ (pathway reviewed in Yu et al., 2015; Holden and Cunningham, 2018).

Accordingly, a hyperactivation of YAP and/or TAZ (and subsequent hyperactivity of the YAP/TAZ-TEAD transcriptional complex) is commonly observed in several human cancers. This is evidenced by the levels and nuclear localization of YAP/TAZ being elevated in many tumors, including breast, lung (e.g., non-small cell; NSCLC), ovarian, colorectal, pancreas, prostate, gastric, esophagus, liver and bone (sarcoma) (Steinhardt et al., 2008; Harvey et al., 2013; Moroishi et al., 2015; extensively reviewed in Zanconato et al., 2016 and references therein).

While genetic alterations of the core Hippo pathway components have thus far been detected with limited frequency in primary samples, the most prominent cancer malignancy associated with inactivating mutations in NF2 or Lats1/2 and associated YAP/TEAD hyperactivity is malignant pleural mesothelioma (MPM) (reviewed in Sekido, 2018). Similarly, a number of human tumors are characterized by amplification of YAP at the 11q22.1 locus (e.g., hepatocellular carcinomas, medulloblastomas, esophageal squamous cell carcinomas), TAZ (WWTR1) at the 3q25.1 locus (e.g., rhabdomyosarcomas, triple negative breast cancer) or gene fusions involving YAP or TAZ (epithelioid hemangioendotheliomas, ependymal tumors) (reviewed in Yu et al., 2015 and references therein). As is the case for MPM, such tumors are also anticipated to depend on their elevated YAP/TAZ-TEAD activity.

Disruption of the YAP/TAZ-TEAD PPI as the most distal effector node of the Hippo pathway is anticipated to abolish the oncogenic potential of this complex.

Notably, tumor cells with activated YAP/TAZ-TEAD display resistance to chemotherapeutic drugs, possibly related to YAP/TAZ conferring cancer stem cell-like characteristics. Moreover, YAP/TAZ-TEAD activation also confers resistance to molecularly targeted therapies, such as BRAF, MEK or EGFR inhibitors, as reported from the outcome of various genetic and pharmacological screens (Kapoor et al., 2014; Shao et al., 2014; Lin et al., 2015). This in turn suggests that inhibiting YAP/TAZ-TEAD activity—either in parallel or sequentially to other cancer treatments—may provide a beneficial therapeutic impact by reducing growth of tumors resistant to other treatments. The inhibition of YAP/TAZ-TEAD activity upon PPI disruption with above mentioned polymorphic forms may also blunt the tumor's escape from immune surveillance. This is, for instance, evidenced by reported data on YAP promoting the expression of chemokine CXCL5 which results in the recruitment of myeloid cells that suppress T-cells (Wang et al., 2016). YAP in Tregs (regulatory T-cells) has also been demonstrated to support FOXP3 expression via activin signaling and Treg function. Accordingly, YAP deficiency results in dysfunctional Tregs which are no longer able to suppress antitumor immunity. Selective inhibition of YAP/TEAD activity may therefore contribute to bolster antitumor immunity by preventing Treg function (Ni et al., 2018). Recent literature also suggests that YAP upregulates PD-L1 expression and by this mechanism directly mediates evasion of cytotoxic T-cell immune responses, for instance in BRAF inhibitor-resistant melanoma cells (Kim et al., 2018).

### See for Example:

Yu, F-X., Zhao, B. and Guan, K.-L. (2015). Hippo pathway in organ size control, tissue homeostasis, and cancer. Cell, 163, 811-828.
Holden, J. K. and Cunningham, C. N. (2018). Targeting the Hippo pathway and cancer through the TEAD family of transcription factors. Cancers (Basel), 10, E81.
Steinhardt, A. A., Gayyed, M. F., Klein, A. P., Dong, J., Maitra, A., Pan, D., Montgomery, E. A., Anders, R. A. (2008). Expression of Yes-associated protein in common solid tumors. Hum. Pathol., 39, 1582-1589.
Harvey, K. F., Zhang, X., and Thomas, D. M. (2013). The Hippo pathway and human cancer. Nat. Rev. Cancer, 13, 246-257.
Moroishi, T., Hansen, C. G., and Guan, K.-L. (2015). Nat. Rev. Cancer, 15, 73-79.
Zanconato, F., Cordenonsi, M., and Piccolo, S. (2016). YAP/TAZ at the roots of cancer. Cancer Cell, 29, 783-803.
Sekido, Y. (2018). Cancers (Basel), 10, E90.
Kapoor, A., Yao, W., Ying, H., Hua, S., Liewen, A., Wang, Q., Zhong, Y., Wu, C. J., Sadanandam, A., Hu, B. et al. (2014). Yap1 activation enables bypass of oncogenic Kras addiction in pancreatic cancer. Cell, 158, 185-197.
Shao, D. D., Xue, W., Krall, E. B., Bhutkar, A., Piccioni, F., Wang, X., Schinzel, A. C., Sood, S., Rosenbluh, J., Kim, J. W., et al. (2014). KRAS and YAP1 converge to regulate EMT and tumor survival. Cell, 158, 171-184.
Lin, L., Sabnis, A. J., Chan, E., Olivas, V., Cade, L., Pazarentzos, E., Asthana, S., Neel, D., Yan, J. J., Lu, X. et al. (2015). The Hippo effector YAP promotes resistance to RAF- and MEK-targeted cancer therapies. Nat. Genet., 47, 250-256.
Wang, G., Lu, X., Dey, P., Deng, P., Wu, C. C., Jiang, S., Fang, Z., Zhao, K., Konaprathi, R., Hua, S., et al. (2016). Cancer Discov., 6, 80-95.
Ni, X., Tao, J., Barbi, J., Chen, Q., Park B. V., Li, Z., Zhang, N., Lebid, A., Ramaswamy, A., Wei, P., et al. (2018). YAP is essential for Treg-mediated suppression of antitumor immunity. Cancer Discov., 8, 1026-1043.
Kim, M. H., Kim, C. G., Kim, S. K., Shin, S. J., Choe, E. A., Park, S. H., Shin, E. C., and Kim, J. (2018). Cancer Immunol Res., 6, 255-266.

Solid state form of the active pharmaceutical ingredient (API) of a particular drug is often an important determinant of the drug's ease of preparation, hygroscopicity, stability, solubility, storage stability, ease of formulation, rate of dissolution in gastrointestinal fluids and in vivo bioavailability. Crystalline forms occur where the same composition of matter crystallizes in a different lattice arrangement resulting in different thermodynamic properties and stabilities specific to the particular crystalline form. Crystalline forms may also include different hydrates or solvates of the same compound. In deciding which form is preferable, the numerous properties of the forms are compared and the preferred form chosen based on the many physical property variables. It is entirely possible that one form can be preferable in some circumstances where certain aspects such as ease of preparation, stability, etc. are deemed to be critical. In other situations, a different form may be preferred for greater dissolution rate and/or superior bioavailability.

Therefore, this ability of a chemical substance to crystallize in more than one crystalline form can have a profound effect on the shelf life, solubility, formulation properties, and processing properties of a drug. In addition, the action of a drug can be affected by the polymorphism of the drug molecule. Different polymorphs can have different rates of uptake in the body, leading to lower or higher biological activity than desired. In extreme cases, an undesired polymorph can even show toxicity. The occurrence of an unknown crystalline form during manufacture can have a significant impact.

It is not yet possible to predict whether a particular compound or salt of a compound will form polymorphs, whether any such polymorphs will be suitable for commercial use in a therapeutic composition, or which polymorphs will display such desirable properties.

### SUMMARY

The polymorphic forms of this invention are designed and optimized to bind to TEADs and selectively disrupt their interaction with YAP and TAZ, which is believed to result in drugs useful in the treatment of above-mentioned cancers. In particular, such cancers may be characterized by (but not restricted to) some of the described aberrations. In certain aspects, advantages of the polymorphic forms of the invention include improved stability, hygroscopicity and morphology (which can improves flow properties).

There is a need in the art for new polymorphic crystalline forms of 4-((2S,4S)-5-Chloro-6-fluoro-2-phenyl-2-((S)-pyrrolidin-2-yl)-2,3-dihydrobenzofuran-4-yl)-5-fluoro-6-(2-hydroxyethoxy)-N-methylnicotinamide and 2-((2S,3S,4S)-5-Chloro-6-fluoro-3-methyl-2-((methylamino)methyl)-2-phenyl-2,3-dihydrobenzofuran-4-yl)-3-fluoro-4-methoxybenzamide. Such forms may possess desirable physicochemical properties which are particularly advantageous in drug product development, e.g. which exhibit improved stability, hygroscopicity and/or morphology (so as to improve flow properties).

According to a first aspect of the invention, there is hereby provided a crystalline form of 2-((2S,3S,4S)-5-Chloro-6-fluoro-3-methyl-2-((methylamino)methyl)-2-phenyl-2,3-dihydrobenzofuran-4-yl)-3-fluoro-4-methoxybenzamide (Compound B) or pharmaceutically acceptable solvate and/or salt thereof.

According to a second aspect of the invention, there is hereby provided a crystalline form of 4-((2S,4S)-5-Chloro-6-fluoro-2-phenyl-2-((S)-pyrrolidin-2-yl)-2,3-dihydrobenzofuran-4-yl)-5-fluoro-6-(2-hydroxyethoxy)-N-methylnicotinamide (Compound A) or pharmaceutically acceptable solvate and/or salt thereof.

According to a third aspect of the invention, there is hereby provided a pharmaceutical composition comprising the crystalline form of the first or second aspect of the invention and a pharmaceutically acceptable carrier.

According to a fourth aspect of the invention, there is hereby provided the crystalline form of the first or second aspect of the invention or the pharmaceutical composition of the third aspect of the invention for use as a medicament.

According to a fifth aspect of the invention, there is hereby provided a combination comprising a crystalline form of the first or second aspect of the invention and one or more therapeutically active agents.

According to a sixth aspect of the invention, there is hereby provided the crystalline form of the first or second aspect of the invention or the pharmaceutical composition of the third aspect of the invention for use in treating a disease or condition mediated by YAP overexpression and/or YAP amplification and/or YAP/TAZ-TEAD interaction; or for use in treating a cancer or tumor harboring (i) one or more YAP/TAZ fusions; (ii) one or more NF2/LATS1/LATS2 truncating mutations or deletions; or (iii) one or more functional YAP/TAZ fusions.

According to a seventh aspect of the invention, there is hereby provided a method of treating a disease or condition mediated by YAP overexpression and/or YAP amplification and/or YAP/TAZ-TEAD interaction, a method of treating a cancer or tumor harboring (i) one or more YAP/TAZ fusions; (ii) one or more NF2/LATS1/LATS2 truncating mutations or deletions; or (iii) one or more functional YAP/TAZ fusions; said method comprising administering to a subject in need thereof, a therapeutically effective amount of a crystalline form according to the invention (e.g., the first or second aspect of the invention), the pharmaceutical composition of the third aspect of the invention, or the combination of the fifth aspect of the invention.

### BRIEF DESCRIPTION OF THE DRAWINGS

FIG. 1 is an X-ray powder diffraction pattern of the succinate salt of Compound B (2-((2S,3S,4S)-5-Chloro-6-fluoro-3-methyl-2-((methylamino)methyl)-2-phenyl-2,3-dihydrobenzofuran-4-yl)-3-fluoro-4-methoxybenzamide) at room temperature.

FIG. 2 is a differential scanning calorimetry (DSC) thermogram of the succinate salt of Compound B (2-((2S,3S,4S)-5-Chloro-6-fluoro-3-methyl-2-((methylamino)methyl)-2-phenyl-2,3-dihydrobenzofuran-4-yl)-3-fluoro-4-methoxybenzamide). Differential scanning calorimetry was conducted for each crystalline form using a TA Discovery DSC instrument. For each analysis, 1-3 mg of sample was placed in an aluminum T-zero crucible that closed with a pin-hole lid. The heating rate was 10° C. per minute in the temperature range between 0 and 300° C. Temperatures are reported in degrees Celsius (° C.) and enthalpies are reported in Joules per gram (J/g). Plots are showing endothermic peaks as down. The endothermic melt peak (melting point) was evaluated for extrapolated onset temperature. The accuracy of the measured sample temperature with this method is within about ±1° C., and the heat of fusion can be measured within a relative error of about ±5%.

### Melting Endotherm: Tₒₙₛₑₜ=200.0° C. (Melting Under Decomposition)

FIG. 3 is a thermogravimetric analysis (TGA) diagram of the succinate salt of Compound B (2-((2S,3S,4S)-5-Chloro-6-fluoro-3-methyl-2-((methylamino)methyl)-2-phenyl-2,3-dihydrobenzofuran-4-yl)-3-fluoro-4-methoxybenzamide)TGA curves were obtained using a TA Discovery TGA instrument. For each analysis, 2-10 mg of sample was placed into an aluminum crucible and closed with a pin-hole lid. The TGA curve was measured at a heating rate of 10° C./min between 30-300° C. The LoD (Loss of drying) was calculated between 30° C. and 200° C. The weight loss is plotted against the measured sample temperature. Temperatures are reported in degrees Celsius (° C.) and weight loss in %.

### Loss of Drying: LoD=0.45%

FIG. 4 is an X-ray powder diffraction pattern of the L-malate salt of Compound B (2-((2S,3S,4S)-5-Chloro-6-fluoro-3-methyl-2-((methylamino)methyl)-2-phenyl-2,3-dihydrobenzofuran-4-yl)-3-fluoro-4-methoxybenzamide) at room temperature.

FIG. 5 is a differential scanning calorimetry (DSC) thermogram of the L-malate salt of Compound B (2-((2S,3S,4S)-5-Chloro-6-fluoro-3-methyl-2-((methylamino)methyl)-2-phenyl-2,3-dihydrobenzofuran-4-yl)-3-fluoro-4-methoxybenzamide) Differential scanning calorimetry was conducted for each crystalline form using a TA Discovery DSC instrument. For each analysis, 1-3 mg of sample was placed in an aluminum T-zero crucible that closed with a pin-hole lid. The heating rate was 10° C. per minute in the temperature range between 0 and 300° C. Temperatures are reported in degrees Celsius (° C.) and enthalpies are reported in Joules per gram (J/g). Plots are showing endothermic peaks as down. The endothermic melt peak (melting point) was evaluated for extrapolated onset temperature. The accuracy of the measured sample temperature with this method is within about ±1° C., and the heat of fusion can be measured within a relative error of about ±5%.

### Melting Endotherm: Tₒₙₛₑₜ=195.4° C. (Melting Under Decomposition)

FIG. 6 is a thermogravimetric analysis (TGA) diagram of the L-malate of Compound B (2-((2S,3S,4S)-5-Chloro-6-fluoro-3-methyl-2-((methylamino)methyl)-2-phenyl-2,3-dihydrobenzofuran-4-yl)-3-fluoro-4-methoxybenzamide) TGA curves were obtained using a TA Discovery TGA instrument. For each analysis, 2-10 mg of sample was placed into an aluminum crucible and closed with a pin-hole lid. The TGA curve was measured at a heating rate of 10° C./min between 30-300° C. The LoD (Loss of drying) was calculated between 30° C. and 200° C. The weight loss is plotted against the measured sample temperature. Temperatures are reported in degrees Celsius (° C.) and weight loss in %.

### Loss of Drying: LoD=0.81%

FIG. 7 is an X-ray powder diffraction pattern of the L-lactate salt of Compound B (2-((2S,3S,4S)-5-Chloro-6-fluoro-3-methyl-2-((methylamino)methyl)-2-phenyl-2,3-dihydrobenzofuran-4-yl)-3-fluoro-4-methoxybenzamide) at room temperature.

FIG. 8 is a differential scanning calorimetry (DSC) thermogram of the L-lactate salt of Compound B (2-((2S,3S,4S)-5-Chloro-6-fluoro-3-methyl-2-((methylamino)methyl)-2-phenyl-2,3-dihydrobenzofuran-4-yl)-3-fluoro-4-methoxybenzamide) Differential scanning calorimetry was conducted for each crystalline form using a TA Discovery DSC instrument. For each analysis, 1-3 mg of sample was placed in an aluminum T-zero crucible that closed with a pin-hole lid. The heating rate was 10° C. per minute in the temperature range between 0 and 300° C. Temperatures are reported in degrees Celsius (° C.) and enthalpies are reported in Joules per gram (J/g). Plots are showing endothermic peaks as down. The endothermic melt peak (melting point) was evaluated for extrapolated onset temperature. The accuracy of the measured sample temperature with this method is within about ±1° C., and the heat of fusion can be measured within a relative error of about ±5%.

### Melting Endotherm: Tₒₙₛₑₜ=207.1° C. (Melting Under Decomposition)

FIG. 9 is a thermogravimetric analysis (TGA) diagram of the L-lactate salt of Compound B (2-((2S,3S,4S)-5-Chloro-6-fluoro-3-methyl-2-((methylamino)methyl)-2-phenyl-2,3-dihydrobenzofuran-4-yl)-3-fluoro-4-methoxybenzamide) TGA curves were obtained using a TA Discovery TGA instrument. For each analysis, 2-10 mg of sample was placed into an aluminum crucible and closed with a pin-hole lid. The TGA curve was measured at a heating rate of 10° C./min between 30-300° C. The LoD (Loss of drying) was calculated between 30° C. and 200° C. The weight loss is plotted against the measured sample temperature. Temperatures are reported in degrees Celsius (° C.) and weight loss in %.

### Loss of Drying: LoD=0.91%

FIG. 10 is an X-ray powder diffraction pattern of the benzoate salt of Compound B (2-((2S,3S,4S)-5-Chloro-6-fluoro-3-methyl-2-((methylamino)methyl)-2-phenyl-2,3-dihydrobenzofuran-4-yl)-3-fluoro-4-methoxybenzamide) at room temperature.

FIG. 11 is a differential scanning calorimetry (DSC) thermogram of the benzoate salt of Compound B (2-((2S,3S,4S)-5-Chloro-6-fluoro-3-methyl-2-((methylamino)methyl)-2-phenyl-2,3-dihydrobenzofuran-4-yl)-3-fluoro-4-methoxybenzamide) Differential scanning calorimetry was conducted for each crystalline form using a TA Discovery DSC instrument. For each analysis, 1-3 mg of sample was placed in an aluminum T-zero crucible that closed with a pin-hole lid. The heating rate was 10° C. per minute in the temperature range between 0 and 300° C. Temperatures are reported in degrees Celsius (° C.) and enthalpies are reported in Joules per gram (J/g). Plots are showing endothermic peaks as down. The endothermic melt peak (melting point) was evaluated for extrapolated onset temperature. The accuracy of the measured sample temperature with this method is within about ±1° C., and the heat of fusion can be measured within a relative error of about ±5%.

### Melting Endotherm: Tₒₙₛₑₜ=166.8° C. (Melting Under Decomposition)

FIG. 12 is a thermogravimetric analysis (TGA) diagram of the benzoate salt of Compound B (2-((2S,3S,4S)-5-Chloro-6-fluoro-3-methyl-2-((methylamino)methyl)-2-phenyl-2,3-dihydrobenzofuran-4-yl)-3-fluoro-4-methoxybenzamide) TGA curves were obtained using a TA Discovery TGA instrument. For each analysis, 2-10 mg of sample was placed into an aluminum crucible and closed with a pin-hole lid. The TGA curve was measured at a heating rate of 10° C./min between 30-300° C. The LoD (Loss of drying) was calculated between 30° C. and 170° C. The weight loss is plotted against the measured sample temperature. Temperatures are reported in degrees Celsius (° C.) and weight loss in %.

### Loss of Drying: LoD=0.72%

FIG. 13 is an X-ray powder diffraction pattern of the glutamate salt of Compound B (2-((2S,3S,4S)-5-Chloro-6-fluoro-3-methyl-2-((methylamino)methyl)-2-phenyl-2,3-dihydrobenzofuran-4-yl)-3-fluoro-4-methoxybenzamide) at room temperature.

FIG. 14 is a differential scanning calorimetry (DSC) thermogram of the glutamate salt (of Compound B (2-((2S,3S,4S)-5-Chloro-6-fluoro-3-methyl-2-((methylamino)methyl)-2-phenyl-2,3-dihydrobenzofuran-4-yl)-3-fluoro-4-methoxybenzamide) Differential scanning calorimetry was conducted for each crystalline form using a TA Discovery DSC instrument. For each analysis, 1-3 mg of sample was placed in an aluminum T-zero crucible that closed with a pin-hole lid. The heating rate was 10° C. per minute in the temperature range between 0 and 300° C. Temperatures are reported in degrees Celsius (° C.) and enthalpies are reported in Joules per gram (J/g). Plots are showing endothermic peaks as down. The endothermic melt peak (melting point) was evaluated for extrapolated onset temperature. The accuracy of the measured sample temperature with this method is within about ±1° C., and the heat of fusion can be measured within a relative error of about ±5%.

Melting Endotherm: Tₒₙₛₑₜ=26° C. (Dehydration) and Tₒₙₛₑₜ=158.6° C. (Melting)

FIG. 15 is a thermogravimetric analysis (TGA) diagram of the glutamate salt of Compound B (2-((2S,3S,4S)-5-Chloro-6-fluoro-3-methyl-2-((methylamino)methyl)-2-phenyl-2,3-dihydrobenzofuran-4-yl)-3-fluoro-4-methoxybenzamide) TGA curves were obtained using a TA Discovery TGA instrument. For each analysis, 2-10 mg of sample was placed into an aluminum crucible and closed with a pin-hole lid. The TGA curve was measured at a heating rate of 10° C./min between 30-300° C. The LoD (Loss of drying) was calculated between 30° C. and 135° C. The weight loss is plotted against the measured sample temperature. Temperatures are reported in degrees Celsius (° C.) and weight loss in %.

### Loss of Drying: LoD=1.45%

FIG. 16 is an X-ray powder diffraction pattern of the malate salt of Compound B (2-((2S,3S,4S)-5-Chloro-6-fluoro-3-methyl-2-((methylamino)methyl)-2-phenyl-2,3-dihydrobenzofuran-4-yl)-3-fluoro-4-methoxybenzamide) at room temperature.

FIG. 17 is a differential scanning calorimetry (DSC) thermogram of the malate salt of Compound B (2-((2S,3S,4S)-5-Chloro-6-fluoro-3-methyl-2-((methylamino)methyl)-2-phenyl-2,3-dihydrobenzofuran-4-yl)-3-fluoro-4-methoxybenzamide). Differential scanning calorimetry was conducted for each crystalline form using a TA Discovery DSC instrument. For each analysis, 1-3 mg of sample was placed in an aluminum T-zero crucible that closed with a pin-hole lid. The heating rate was 10° C. per minute in the temperature range between 0 and 300° C. Temperatures are reported in degrees Celsius (° C.) and enthalpies are reported in Joules per gram (J/g). Plots are showing endothermic peaks as down. The endothermic melt peak (melting point) was evaluated for extrapolated onset temperature. The accuracy of the measured sample temperature with this method is within about ±1° C., and the heat of fusion can be measured within a relative error of about ±5%.

### Melting Endotherm: Tₒₙₛₑₜ=204.0° C. (Melting Under Decomposition)

FIG. 18 is a thermogravimetric analysis (TGA) diagram of the malate salt of Compound B (2-((2S,3S,4S)-5-Chloro-6-fluoro-3-methyl-2-((methylamino)methyl)-2-phenyl-2,3-dihydrobenzofuran-4-yl)-3-fluoro-4-methoxybenzamide) TGA curves were obtained using a TA Discovery TGA instrument. For each analysis, 2-10 mg of sample was placed into an aluminum crucible and closed with a pin-hole lid. The TGA curve was measured at a heating rate of 10° C./min between 30-300° C. The LoD (Loss of drying) was calculated between 30° C. and 200° C. The weight loss is plotted against the measured sample temperature. Temperatures are reported in degrees Celsius (° C.) and weight loss in %.

### Loss of Drying: LoD=0.60%

FIG. 19 is an X-ray powder diffraction pattern of the malonate salt (type 1) of Compound B (2-((2S,3S,4S)-5-Chloro-6-fluoro-3-methyl-2-((methylamino)methyl)-2-phenyl-2,3-dihydrobenzofuran-4-yl)-3-fluoro-4-methoxybenzamide) at room temperature.

FIG. 20 is a differential scanning calorimetry (DSC) thermogram of the malonate salt (type 1) of Compound B (2-((2S,3S,4S)-5-Chloro-6-fluoro-3-methyl-2-((methylamino)methyl)-2-phenyl-2,3-dihydrobenzofuran-4-yl)-3-fluoro-4-methoxybenzamide). Differential scanning calorimetry was conducted using a TA Discovery DSC instrument. 1-3 mg of sample was placed in an aluminum T-zero crucible that closed with a pin-hole lid. The heating rate was 10° C. per minute in the temperature range between 0 and 300° C. Temperatures are reported in degrees Celsius (° C.) and enthalpies are reported in Joules per gram (J/g). Plots are showing endothermic peaks as down. The endothermic melt peak (melting point) was evaluated for extrapolated onset temperature. The accuracy of the measured sample temperature with this method is within about ±1° C., and the heat of fusion can be measured within a relative error of about ±5%.

### Melting Endotherm: Tₒₙₛₑₜ=186.6° C. (Melting Under Decomposition)

FIG. 21 is a thermogravimetric analysis (TGA) diagram of the malonate salt (type 1) of Compound B (2-((2S,3S,4S)-5-Chloro-6-fluoro-3-methyl-2-((methylamino)methyl)-2-phenyl-2,3-dihydrobenzofuran-4-yl)-3-fluoro-4-methoxybenzamide). TGA curves were obtained using a TA Discovery TGA instrument. 2-10 mg of sample was placed into an aluminum crucible and closed with a pin-hole lid. The TGA curve was measured at a heating rate of 10° C./min between 30-300° C. The LoD (Loss of drying) was calculated between 30° C. and 182° C. The weight loss is plotted against the measured sample temperature. Temperatures are reported in degrees Celsius (° C.) and weight loss in %.

### Loss of Drying: LoD=0.51%

FIG. 22 is an X-ray powder diffraction pattern of the malonate salt (type 1l) of Compound B (2-((2S,3S,4S)-5-Chloro-6-fluoro-3-methyl-2-((methylamino)methyl)-2-phenyl-2,3-dihydrobenzofuran-4-yl)-3-fluoro-4-methoxybenzamide) at room temperature.

FIG. 23 is a differential scanning calorimetry (DSC) thermogram of the malonate salt (type 1l) of Compound B (2-((2S,3S,4S)-5-Chloro-6-fluoro-3-methyl-2-((methylamino)methyl)-2-phenyl-2,3-dihydrobenzofuran-4-yl)-3-fluoro-4-methoxybenzamide). Differential scanning calorimetry was conducted using a TA Discovery DSC instrument. 1-3 mg of sample was placed in an aluminum T-zero crucible that closed with a pin-hole lid. The heating rate was 10° C. per minute in the temperature range between 0 and 300° C. Temperatures are reported in degrees Celsius (° C.) and enthalpies are reported in Joules per gram (J/g). Plots are showing endothermic peaks as down. The endothermic melt peak (melting point) was evaluated for extrapolated onset temperature. The accuracy of the measured sample temperature with this method is within about ±1° C., and the heat of fusion can be measured within a relative error of about ±5%.

### Melting Endotherm: Tₒₙₛₑₜ=122.2° C. (Melting and Desolvation)

FIG. 24 is a thermogravimetric analysis (TGA) diagram of the malonate salt (type II) of the malonate salt (type II) of Compound B (2-((2S,3S,4S)-5-Chloro-6-fluoro-3-methyl-2-((methylamino)methyl)-2-phenyl-2,3-dihydrobenzofuran-4-yl)-3-fluoro-4-methoxybenzamide). TGA curves were obtained using a TA Discovery TGA instrument. 2-10 mg of sample was placed into an aluminum crucible and closed with a pin-hole lid. The TGA curve was measured at a heating rate of 10° C./min between 30-300° C. The LoD (Loss of drying) was calculated between 30° C. and 200° C. The weight loss is plotted against the measured sample temperature. Temperatures are reported in degrees Celsius (° C.) and weight loss in %.

### Type II: Loss of Drying: LoD=26.7%

FIG. 25 is an X-ray powder diffraction pattern of the mesylate salt of Compound B (2-((2S,3S,4S)-5-Chloro-6-fluoro-3-methyl-2-((methylamino)methyl)-2-phenyl-2,3-dihydrobenzofuran-4-yl)-3-fluoro-4-methoxybenzamide) at room temperature.

FIG. 26 is a differential scanning calorimetry (DSC) thermogram of the mesylate salt of Compound B (2-((2S,3S,4S)-5-Chloro-6-fluoro-3-methyl-2-((methylamino)methyl)-2-phenyl-2,3-dihydrobenzofuran-4-yl)-3-fluoro-4-methoxybenzamide). Differential scanning calorimetry was conducted for each crystalline form using a TA Discovery DSC instrument. For each analysis, 1-3 mg of sample was placed in an aluminum T-zero crucible that closed with a pin-hole lid. The heating rate was 10° C. per minute in the temperature range between 0 and 300° C. Temperatures are reported in degrees Celsius (° C.) and enthalpies are reported in Joules per gram (J/g). Plots are showing endothermic peaks as down. The endothermic melt peak (melting point) was evaluated for extrapolated onset temperature. The accuracy of the measured sample temperature with this method is within about ±1° C., and the heat of fusion can be measured within a relative error of about ±5%.

Melting Endotherm: Tₒₙₛₑₜ=22° C. (Dehydration) and Tₒₙₛₑₜ=267.9° C. (Melting)

FIG. 27 is a thermogravimetric analysis (TGA) diagram of the mesylate salt of Compound B (2-((2S,3S,4S)-5-Chloro-6-fluoro-3-methyl-2-((methylamino)methyl)-2-phenyl-2,3-dihydrobenzofuran-4-yl)-3-fluoro-4-methoxybenzamide). TGA curves were obtained using a TA Discovery TGA instrument. For each analysis, 2-10 mg of sample was placed into an aluminum crucible and closed with a pin-hole lid. The TGA curve was measured at a heating rate of 10° C./min between 30-300° C. The LoD (Loss of drying) was calculated between 30° C. and 100° C. The weight loss is plotted against the measured sample temperature. Temperatures are reported in degrees Celsius (° C.) and weight loss in %.

### Loss of Drying: LoD=0.34%

FIG. 28 is an X-ray powder diffraction pattern of the free form (2-methyl-2-butanol solvate) of Compound B (2-((2S,3S,4S)-5-Chloro-6-fluoro-3-methyl-2-((methylamino)methyl)-2-phenyl-2,3-dihydrobenzofuran-4-yl)-3-fluoro-4-methoxybenzamide) at room temperature.

FIG. 29 is a differential scanning calorimetry (DSC) thermogram of the free form (2-methyl-2-butanol solvate) of Compound B (2-((2S,3S,4S)-5-Chloro-6-fluoro-3-methyl-2-((methylamino)methyl)-2-phenyl-2,3-dihydrobenzofuran-4-yl)-3-fluoro-4-methoxybenzamide) Differential scanning calorimetry was conducted for each crystalline form using a TA Discovery DSC instrument. For each analysis, 1-3 mg of sample was placed in an aluminum T-zero crucible that closed with a pin-hole lid. The heating rate was 10° C. per minute in the temperature range between 0 and 300° C. Temperatures are reported in degrees Celsius (° C.) and enthalpies are reported in Joules per gram (J/g). Plots are showing endothermic peaks as down. The endothermic melt peak (melting point) was evaluated for extrapolated onset temperature. The accuracy of the measured sample temperature with this method is within about ±1° C., and the heat of fusion can be measured within a relative error of about ±5%.

### Melting Endotherm: Tₒₙₛₑₜ=68° C. (Melt and Desolvation)

FIG. 30 is a thermogravimetric analysis (TGA) diagram of the free form (2-methyl-2-butanol solvate) of Compound B (2-((2S,3S,4S)-5-Chloro-6-fluoro-3-methyl-2-((methylamino)methyl)-2-phenyl-2,3-dihydrobenzofuran-4-yl)-3-fluoro-4-methoxybenzamide). TGA curves were obtained using a TA Discovery TGA instrument. For each analysis, 2-10 mg of sample was placed into an aluminum crucible and closed with a pin-hole lid. The TGA curve was measured at a heating rate of 10° C./min between 30-300° C. The LoD (Loss of drying) was calculated between 30° C. and 100° C. The weight loss is plotted against the measured sample temperature. Temperatures are reported in degrees Celsius (° C.) and weight loss in %.

### Loss of Drying: LoD=5.91%

FIG. 31 is an X-ray powder diffraction pattern of the “Modification A” free form of Compound A (4-((2S,4S)-5-Chloro-6-fluoro-2-phenyl-2-((S)-pyrrolidin-2-yl)-2,3-dihydrobenzofuran-4-yl)-5-fluoro-6-(2-hydroxyethoxy)-N-methylnicotinamide) at room temperature.

FIG. 32 is a differential scanning calorimetry (DSC) thermogram of the “Modification A” free form of Compound A (4-((2S,4S)-5-Chloro-6-fluoro-2-phenyl-2-((S)-pyrrolidin-2-yl)-2,3-dihydrobenzofuran-4-yl)-5-fluoro-6-(2-hydroxyethoxy)-N-methylnicotinamide). Differential scanning calorimetry was conducted for each crystalline form using a TA Discovery DSC instrument. For each analysis, 1-3 mg of sample was placed in an aluminum T-zero crucible that closed with a pin-hole lid. The heating rate was 10° C. per minute in the temperature range between 0 and 300° C. Temperatures are reported in degrees Celsius (° C.) and enthalpies are reported in Joules per gram (J/g). Plots are showing endothermic peaks as down. The endothermic melt peak (melting point) was evaluated for extrapolated onset temperature. The accuracy of the measured sample temperature with this method is within about ±1° C., and the heat of fusion can be measured within a relative error of about ±5%.

### Melting Endotherm: Tₒₙₛₑₜ=117.5° C. (Melt)

FIG. 33 is a thermogravimetric analysis (TGA) diagram of the “Modification A” free form of Compound A (4-((2S,4S)-5-Chloro-6-fluoro-2-phenyl-2-((S)-pyrrolidin-2-yl)-2,3-dihydrobenzofuran-4-yl)-5-fluoro-6-(2-hydroxyethoxy)-N-methylnicotinamide). TGA curves were obtained using a TA Discovery TGA instrument. For each analysis, 2-10 mg of sample was placed into an aluminum crucible and closed with a pin-hole lid. The TGA curve was measured at a heating rate of 10° C./min between 30-300° C. The LoD (Loss of drying) was calculated between 27° C. and 110° C. The weight loss is plotted against the measured sample temperature. Temperatures are reported in degrees Celsius (° C.) and weight loss in %.

### Loss of Drying: LoD=0.38%

FIG. 34 is an X-ray powder diffraction pattern of the 4-hydroxybenzoate salt of Compound A (4-((2S,4S)-5-Chloro-6-fluoro-2-phenyl-2-((S)-pyrrolidin-2-yl)-2,3-dihydrobenzofuran-4-yl)-5-fluoro-6-(2-hydroxyethoxy)-N-methylnicotinamide) at room temperature.

FIG. 35 is a differential scanning calorimetry (DSC) thermogram of the 4-hydroxybenzoate salt of Compound A (4-((2S,4S)-5-Chloro-6-fluoro-2-phenyl-2-((S)-pyrrolidin-2-yl)-2,3-dihydrobenzofuran-4-yl)-5-fluoro-6-(2-hydroxyethoxy)-N-methylnicotinamide). Differential scanning calorimetry was conducted for each crystalline form using a TA Discovery DSC instrument. For each analysis, 1-3 mg of sample was placed in an aluminum T-zero crucible that closed with a pin-hole lid. The heating rate was 10° C. per minute in the temperature range between 0 and 300° C. Temperatures are reported in degrees Celsius (° C.) and enthalpies are reported in Joules per gram (J/g). Plots are showing endothermic peaks as down. The endothermic melt peak (melting point) was evaluated for extrapolated onset temperature. The accuracy of the measured sample temperature with this method is within about ±1° C., and the heat of fusion can be measured within a relative error of about ±5%.

Melting Endotherm: Tₒₙₛₑₜ=216.7° C. (Melt with Decomposition)

FIG. 36 is a thermogravimetric analysis (TGA) diagram of the 4-hydroxybenzoate salt of Compound A (4-((2S,4S)-5-Chloro-6-fluoro-2-phenyl-2-((S)-pyrrolidin-2-yl)-2,3-dihydrobenzofuran-4-yl)-5-fluoro-6-(2-hydroxyethoxy)-N-methylnicotinamide). TGA curves were obtained using a TA Discovery TGA instrument. For each analysis, 2-10 mg of sample was placed into an aluminum crucible and closed with a pin-hole lid. The TGA curve was measured at a heating rate of 10° C./min between 30-300° C. The LoD (Loss of drying) was calculated between 27° C. and 110° C. The weight loss is plotted against the measured sample temperature. Temperatures are reported in degrees Celsius (° C.) and weight loss in %.

### Loss of Drying: LoD=0.46%

FIG. 37 is an X-ray powder diffraction pattern of the 3,4-dihydroxybenzoate salt of Compound A (4-((2S,4S)-5-Chloro-6-fluoro-2-phenyl-2-((S)-pyrrolidin-2-yl)-2,3-dihydrobenzofuran-4-yl)-5-fluoro-6-(2-hydroxyethoxy)-N-methylnicotinamide) at room temperature.

FIG. 38 is a the differential scanning calorimetry (DSC) thermogram of the 3,4-dihydroxybenzoate salt of Compound A (4-((2S,4S)-5-Chloro-6-fluoro-2-phenyl-2-((S)-pyrrolidin-2-yl)-2,3-dihydrobenzofuran-4-yl)-5-fluoro-6-(2-hydroxyethoxy)-N-methylnicotinamide). Differential scanning calorimetry was conducted for each crystalline form using a TA Discovery DSC instrument. For each analysis, 1-3 mg of sample was placed in an aluminum T-zero crucible that closed with a pin-hole lid. The heating rate was 10° C. per minute in the temperature range between 0 and 300° C. Temperatures are reported in degrees Celsius (° C.) and enthalpies are reported in Joules per gram (J/g). Plots are showing endothermic peaks as down. The endothermic melt peak (melting point) was evaluated for extrapolated onset temperature. The accuracy of the measured sample temperature with this method is within about ±1° C., and the heat of fusion can be measured within a relative error of about ±5%.

Melting Endotherm: Tₒₙₛₑₜ=29° C. (Dehydration) Tₒₙₛₑₜ=216.5° C. (Melt with Decomposition)

FIG. 39 is a thermogravimetric analysis (TGA) diagram of the 3,4-dihydroxybenzoate salt of Compound A (4-((2S,4S)-5-Chloro-6-fluoro-2-phenyl-2-((S)-pyrrolidin-2-yl)-2,3-dihydrobenzofuran-4-yl)-5-fluoro-6-(2-hydroxyethoxy)-N-methylnicotinamide). TGA curves were obtained using a TA Discovery TGA instrument. For each analysis, 2-10 mg of sample was placed into an aluminum crucible and closed with a pin-hole lid. The TGA curve was measured at a heating rate of 10° C./min between 30-300° C. The LoD (Loss of drying) was calculated between 26° C. and 80° C. The weight loss is plotted against the measured sample temperature. Temperatures are reported in degrees Celsius (° C.) and weight loss in %.

### Loss of Drying: LoD=1.63%

It should be understood that in the X-ray powder diffraction spectra or pattern that there is inherent variability in the values measured in degrees 2θ (°2θ) as a result of, for example, instrumental variation (including differences between instruments). As such, it should be understood that there is a variability of up to ±0.2°2θ in XRPD peak measurements and yet such peak values would still be considered to be representative of a particular solid state form of the crystalline materials described herein. It should also be understood that other measured values from XRPD experiments and DSC/TGA experiments, such as relative intensity and water content, can vary as a result of, for example, sample preparation and/or storage and/or environmental conditions, and yet the measured values will still be considered to be representative of a particular solid state form of the crystalline materials described herein.

### DETAILED DESCRIPTION OF THE INVENTION

There is a need in the art for new polymorphic crystalline forms of 4-((2S,4S)-5-Chloro-6-fluoro-2-phenyl-2-((S)-pyrrolidin-2-yl)-2,3-dihydrobenzofuran-4-yl)-5-fluoro-6-(2-hydroxyethoxy)-N-methylnicotinamide and 2-((2S,3S,4S)-5-Chloro-6-fluoro-3-methyl-2-((methylamino)methyl)-2-phenyl-2,3-dihydrobenzofuran-4-yl)-3-fluoro-4-methoxybenzamide. Such forms may possess desirable physicochemical properties which are particularly advantageous in drug product development, e.g. which exhibit improved stability, hygroscopicity and/or morphology (so as to improve flow properties).

The invention therefore provides the following numbered embodiments:

Embodiment 1. A crystalline form of 2-((2S,3S,4S)-5-Chloro-6-fluoro-3-methyl-2-((methylamino)methyl)-2-phenyl-2,3-dihydrobenzofuran-4-yl)-3-fluoro-4-methoxybenzamide (Compound B) or pharmaceutically acceptable solvate and/or salt thereof.

Embodiment 2. A crystalline form of 4-((2S,4S)-5-Chloro-6-fluoro-2-phenyl-2-((S)-pyrrolidin-2-yl)-2,3-dihydrobenzofuran-4-yl)-5-fluoro-6-(2-hydroxyethoxy)-N-methylnicotinamide (Compound A) or pharmaceutically acceptable solvate and/or salt thereof.

Embodiment 3. The crystalline form according to Embodiment 1, wherein the Compound B is in the form a succinate salt.

Embodiment 4. The crystalline form according to Embodiment 3, characterized by an X-ray powder diffraction pattern comprising peaks at four or more 2θ values (e.g. 5 or more, e.g. 6 or more, e.g. 7 or more, e.g. 8 or more, e.g. 9 or more, e.g. 10 or more, e.g. 11 or more, e.g. 12 or more, e.g. all 13 2θ values) selected from the group consisting of:

Angle (2θ)
11.93° ± 0.2°
13.26° ± 0.2°
13.77° ± 0.2°
15.12° ± 0.2°
16.99° ± 0.2°
17.39° ± 0.2°
19.92° ± 0.2°
20.73° ± 0.2°
21.06° ± 0.2°
23.45° ± 0.2°
24.11° ± 0.2°
26.37° ± 0.2°
26.66° ± 0.2°

wherein the temperature is about room temperature and the radiation used has a wavelength of 1.54060 Å, and preferably wherein the X-ray powder diffraction pattern comprises at least the peaks at 13.26°±0.2°, 16.99°±0.2°, 19.92°±0.2° and 26.66°±0.2°.

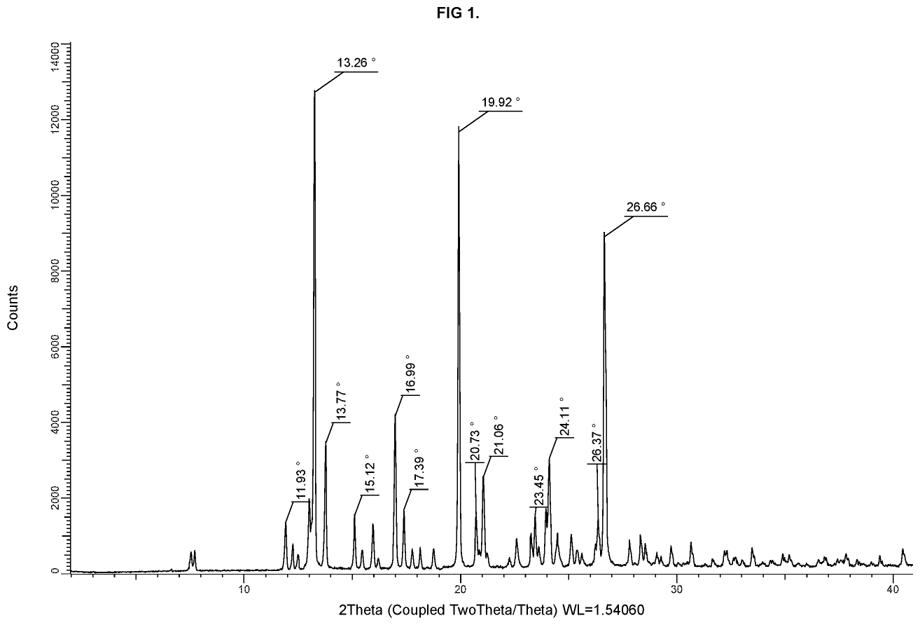

Embodiment 5. The crystalline form according to Embodiment 3 or Embodiment 4, having an X-ray powder diffraction pattern substantially the same as the X-ray powder diffraction spectrum as shown in FIG. 1, at about room temperature wherein the radiation used has a wavelength of 1.54060 Å.

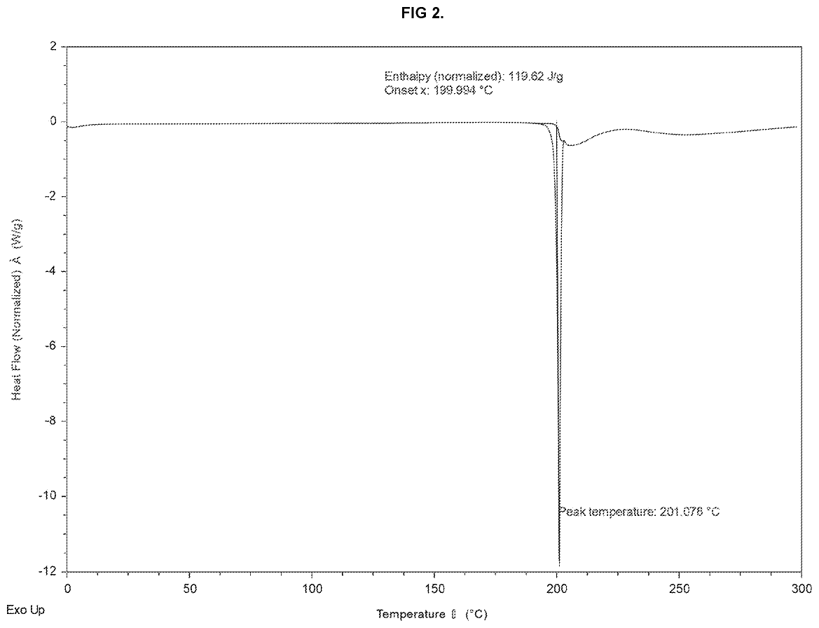

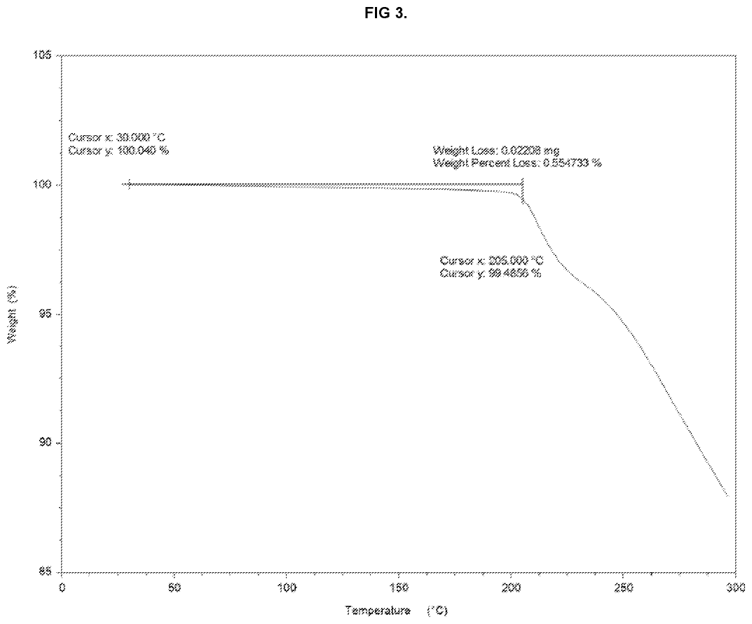

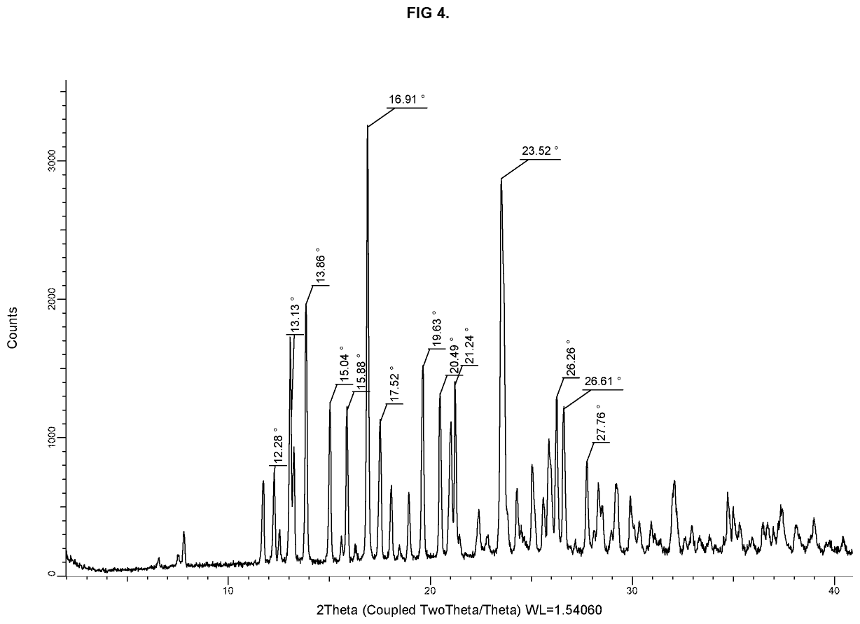

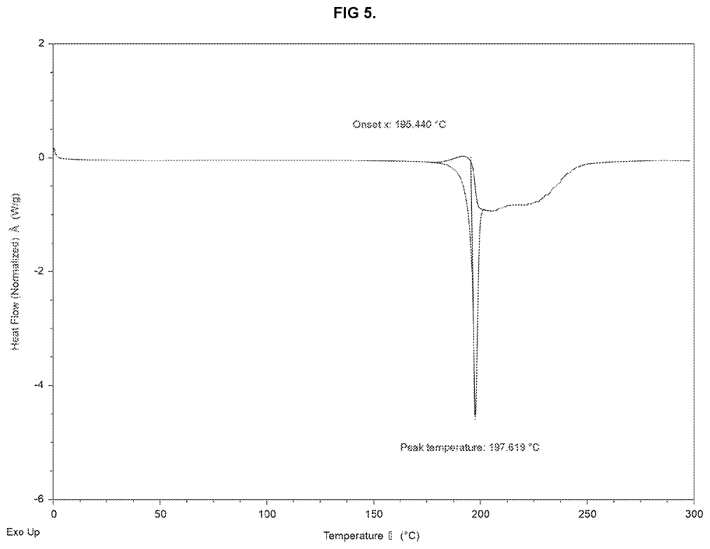

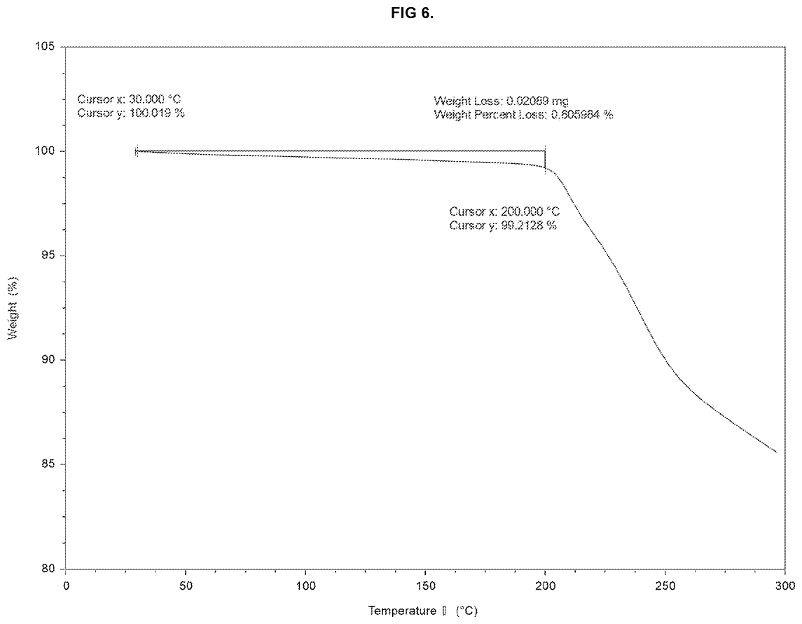

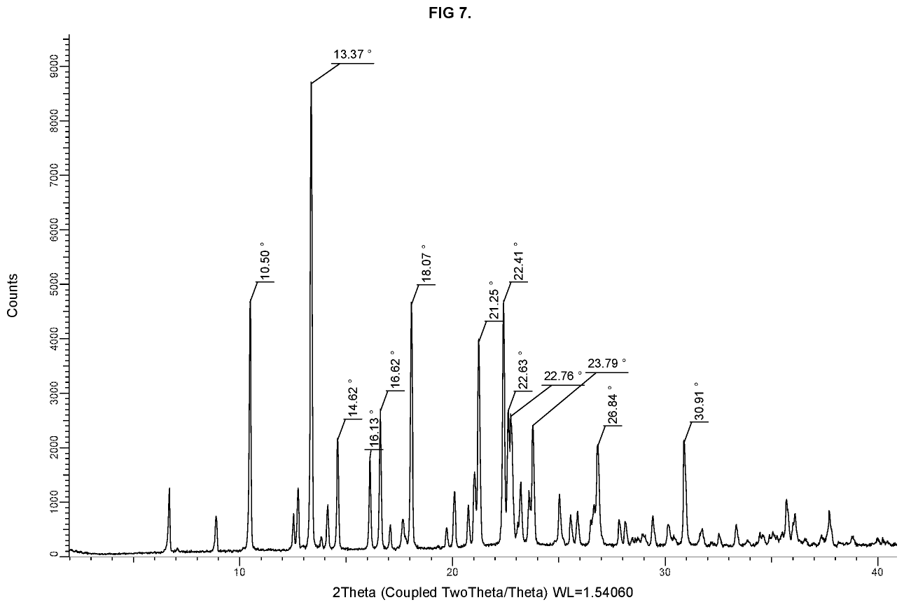

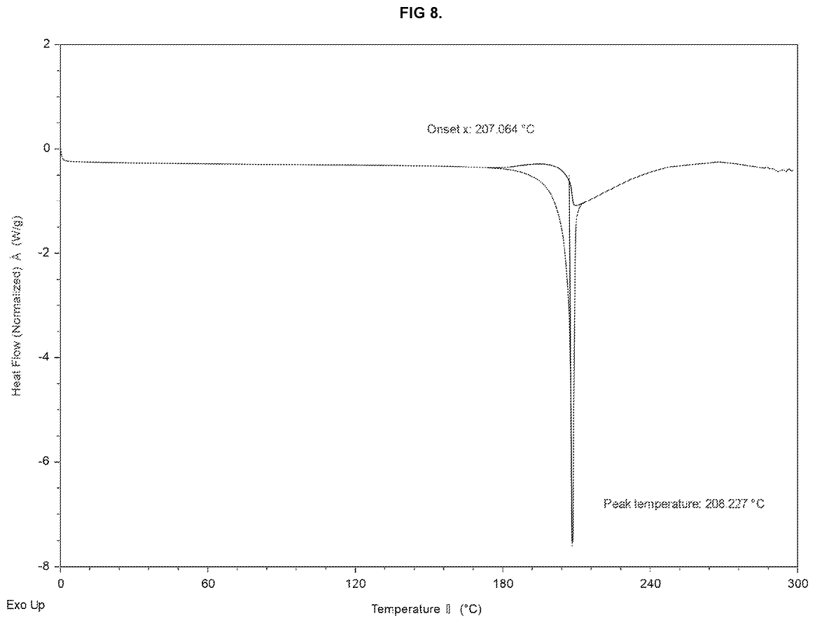

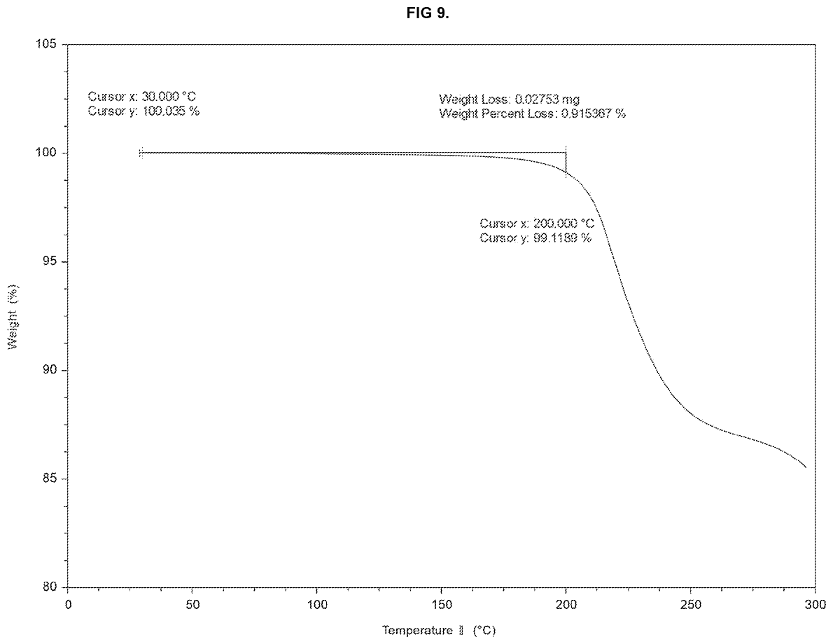

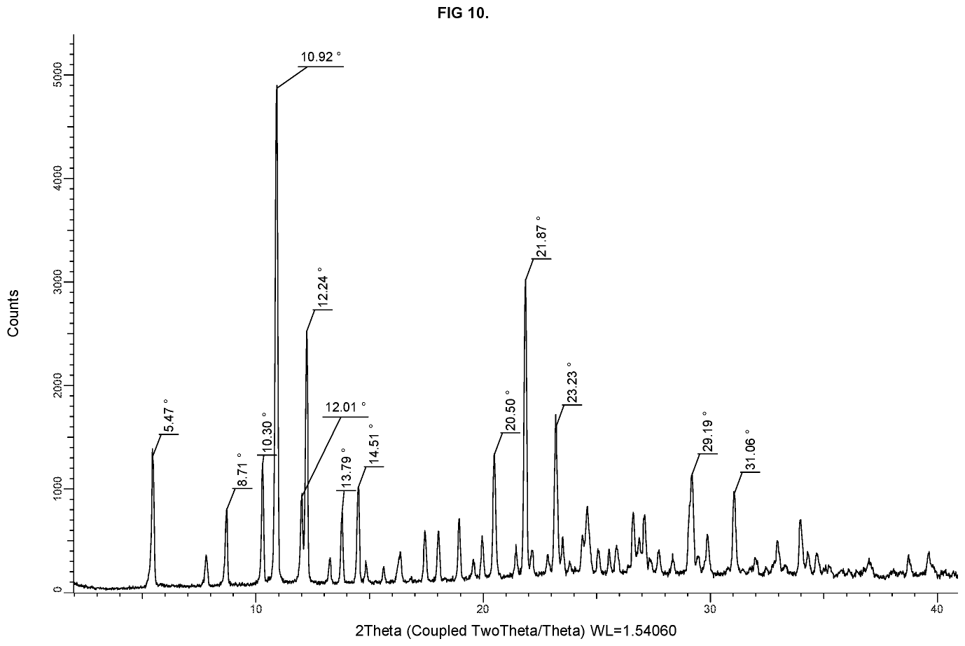

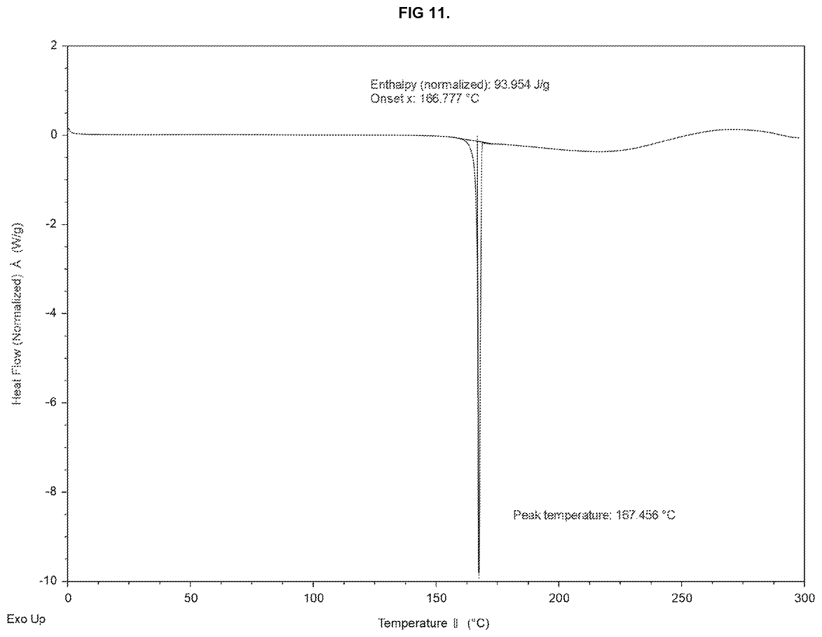

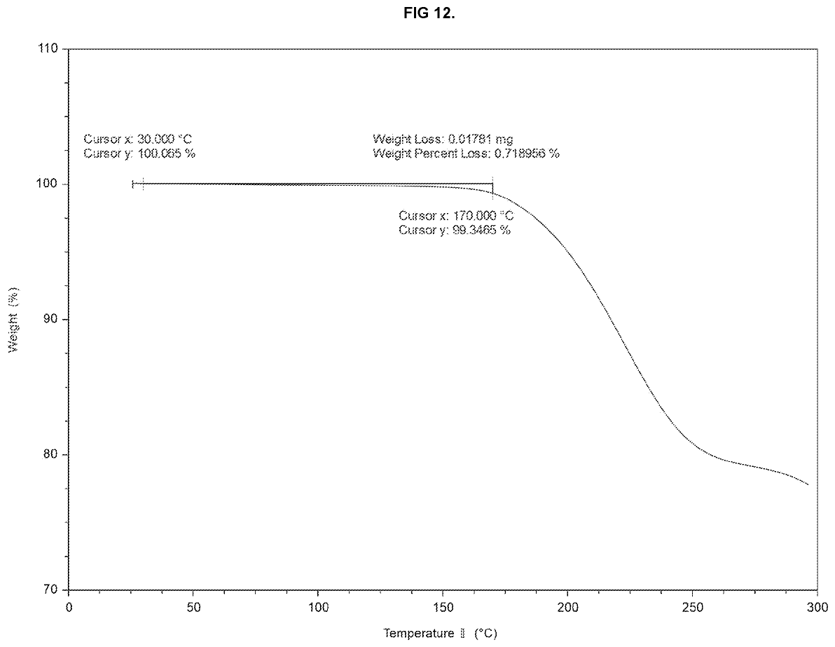

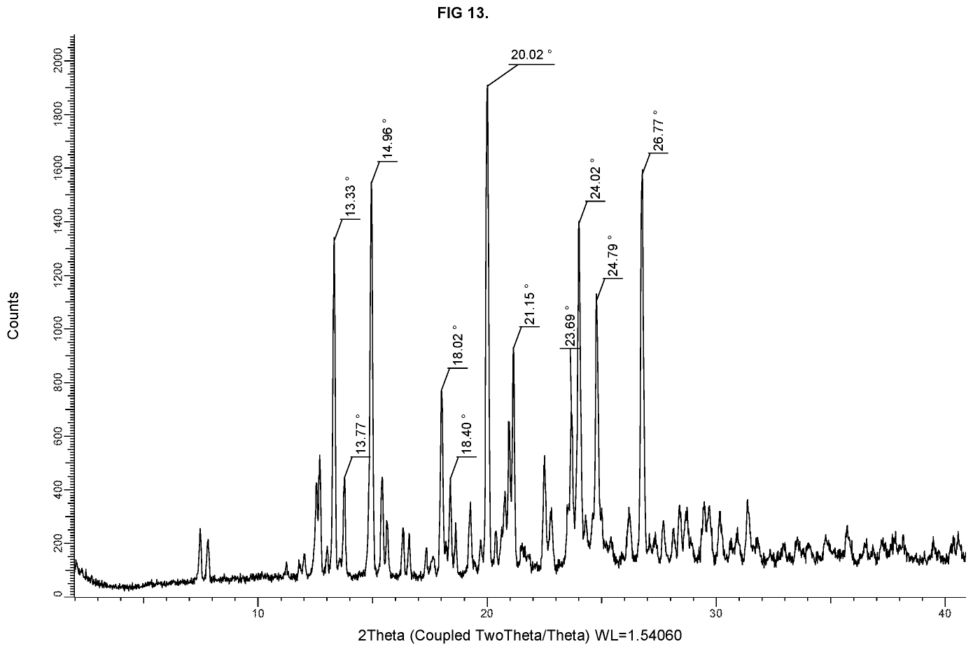

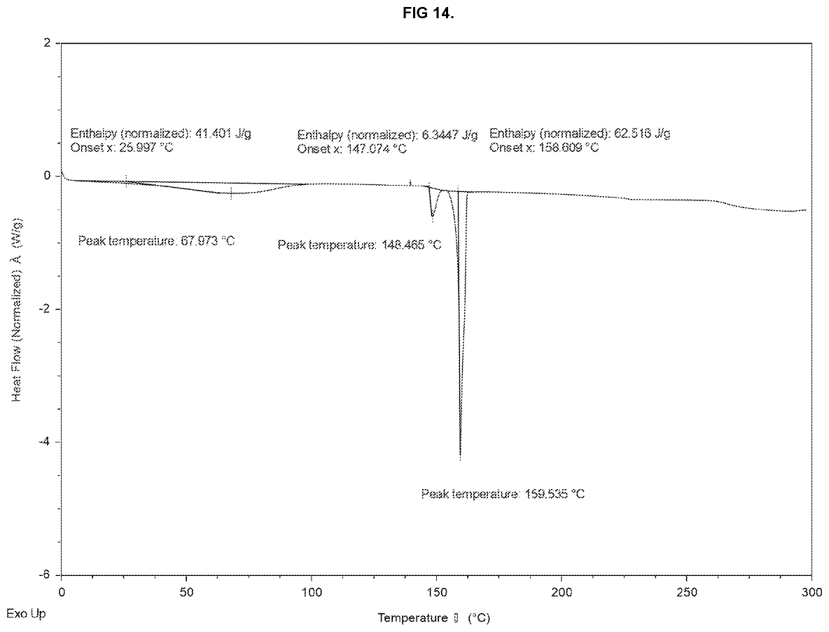

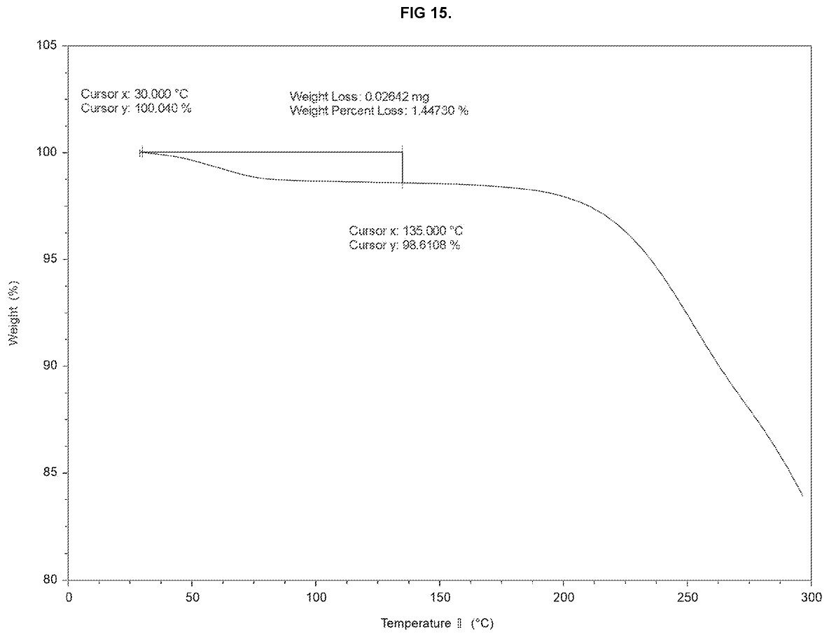

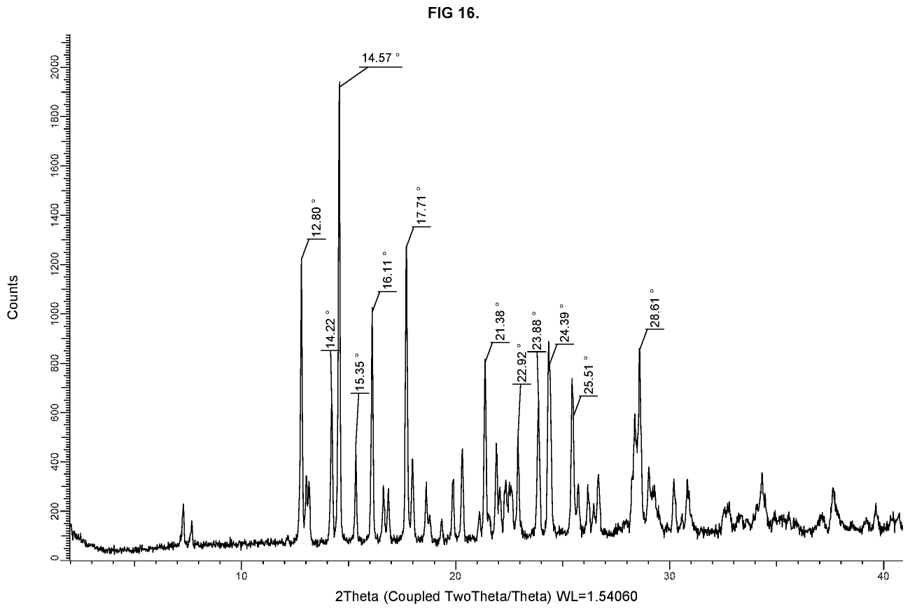

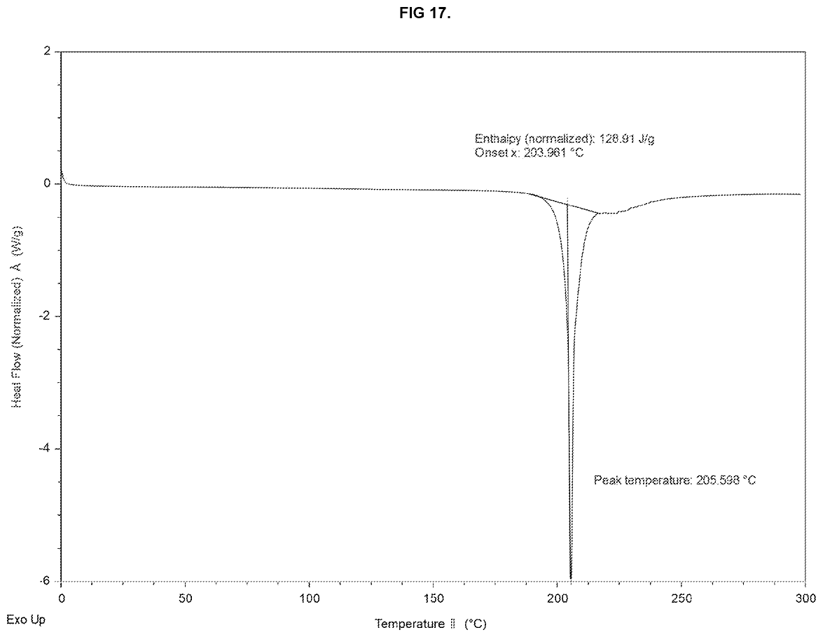

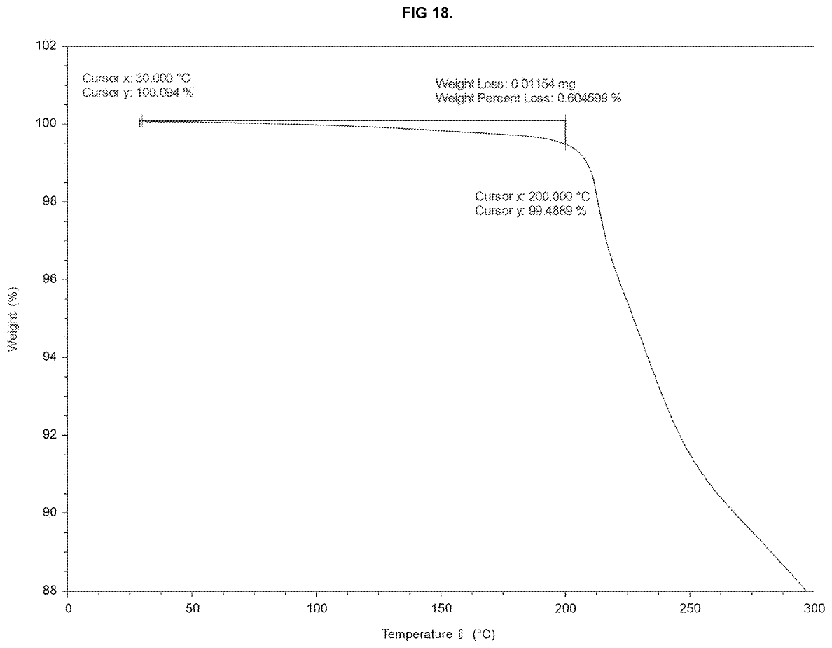

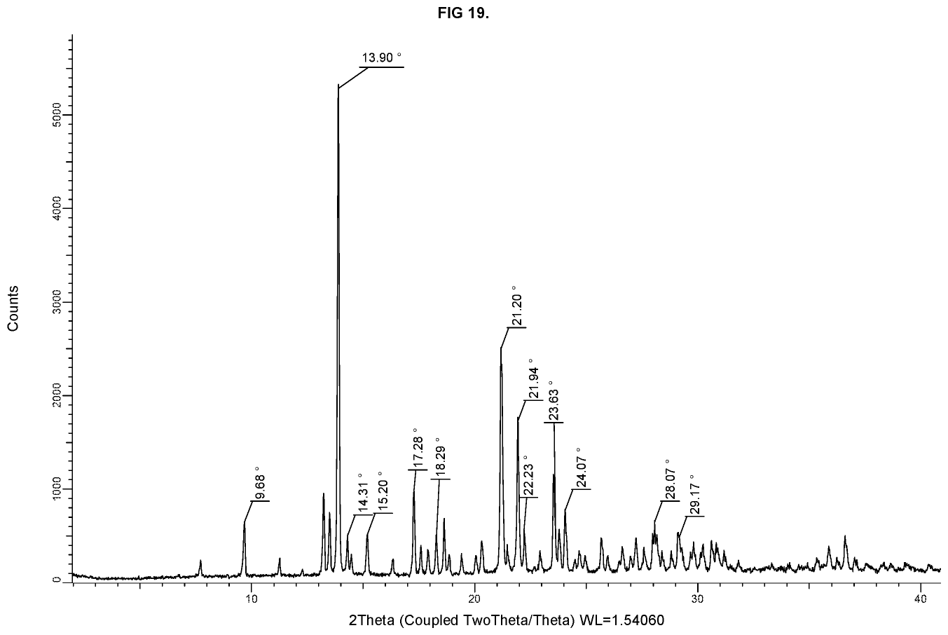

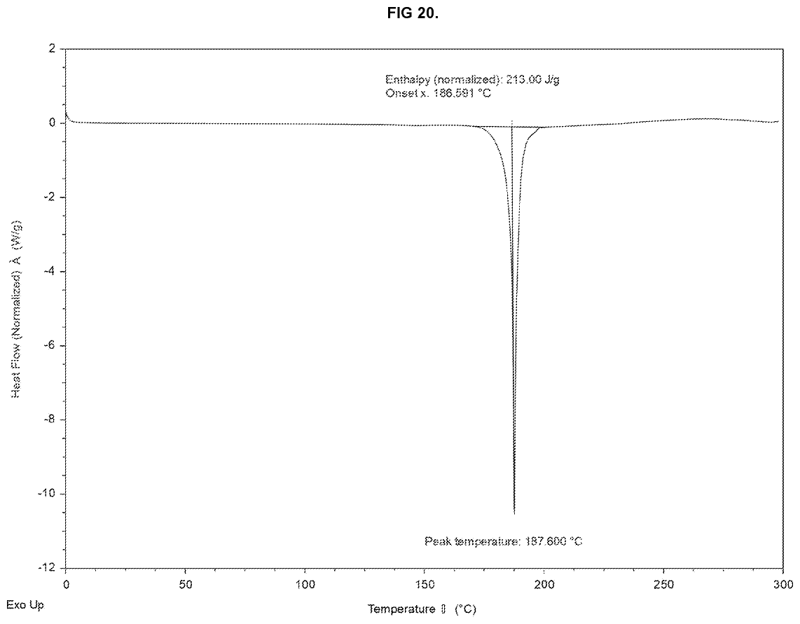

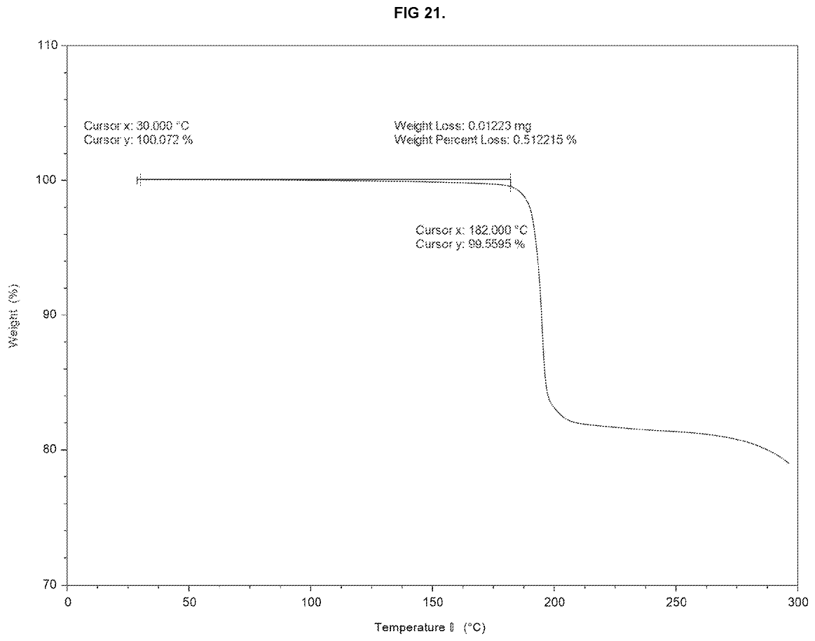

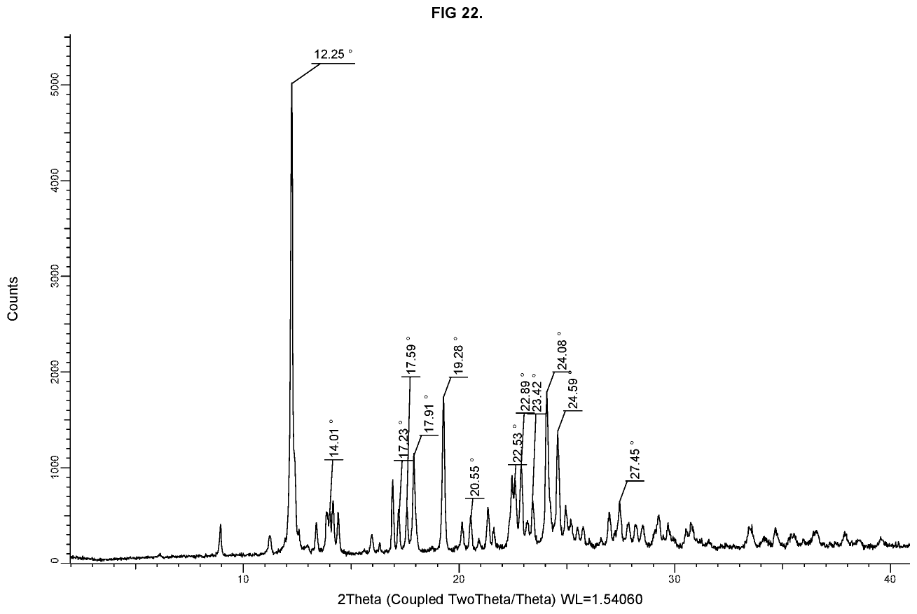

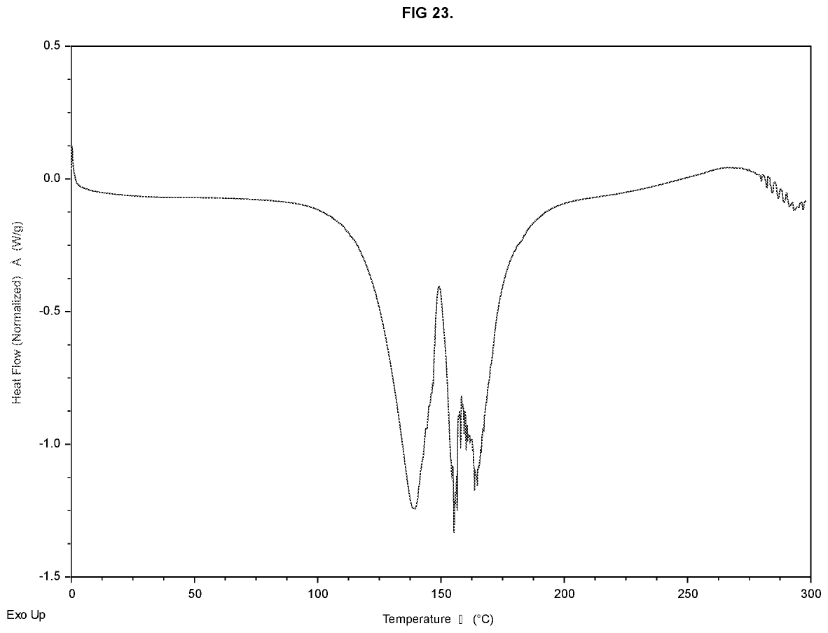

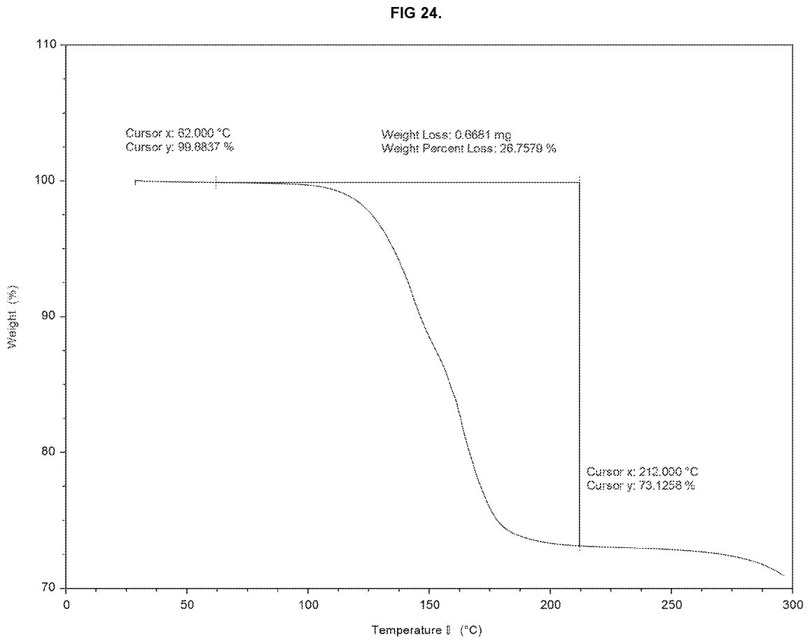

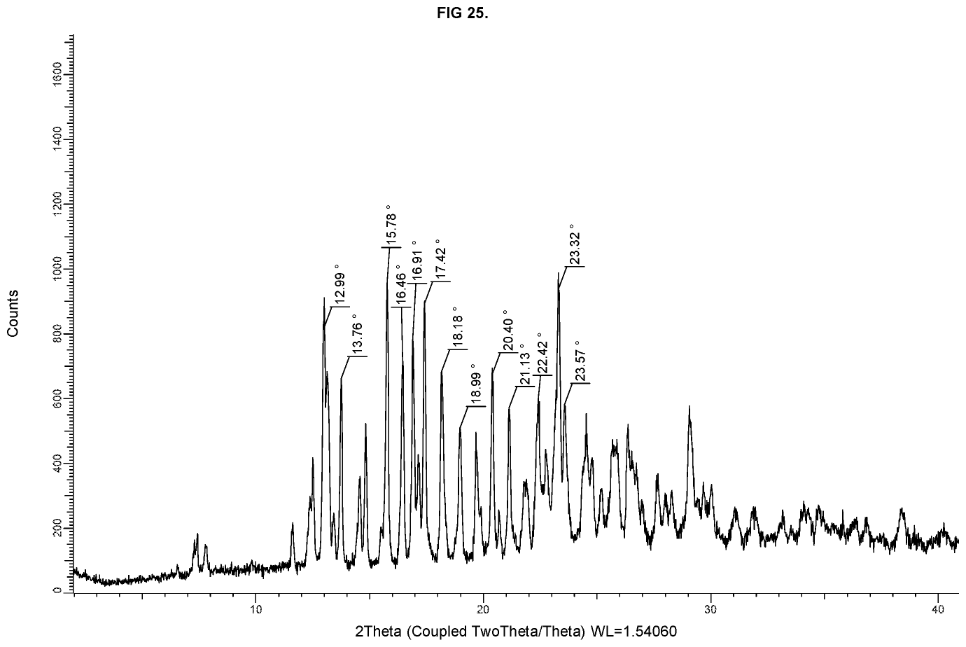

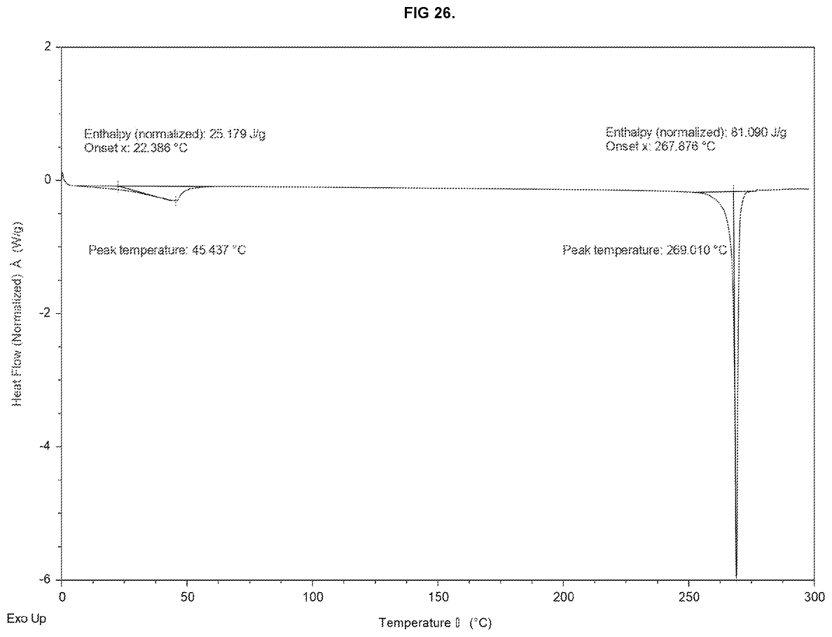

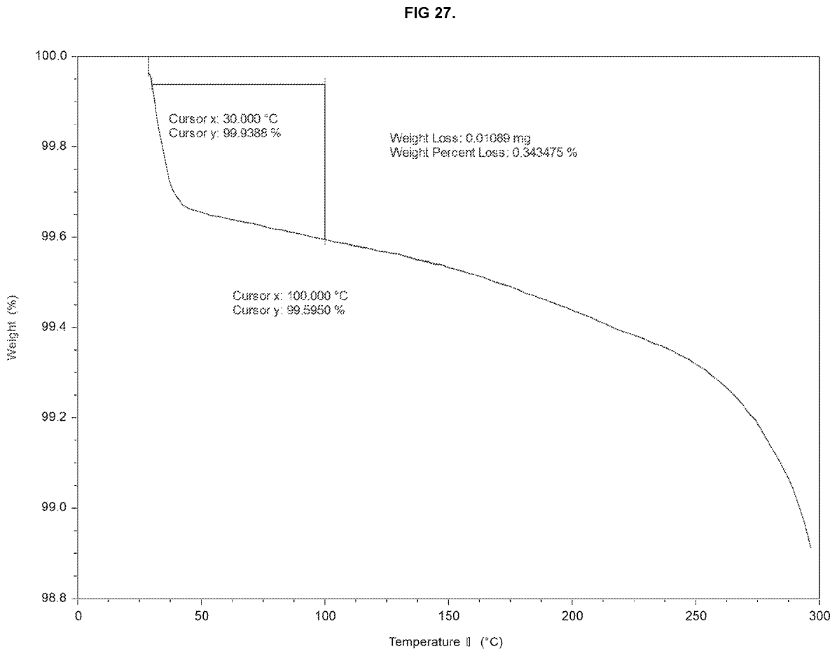

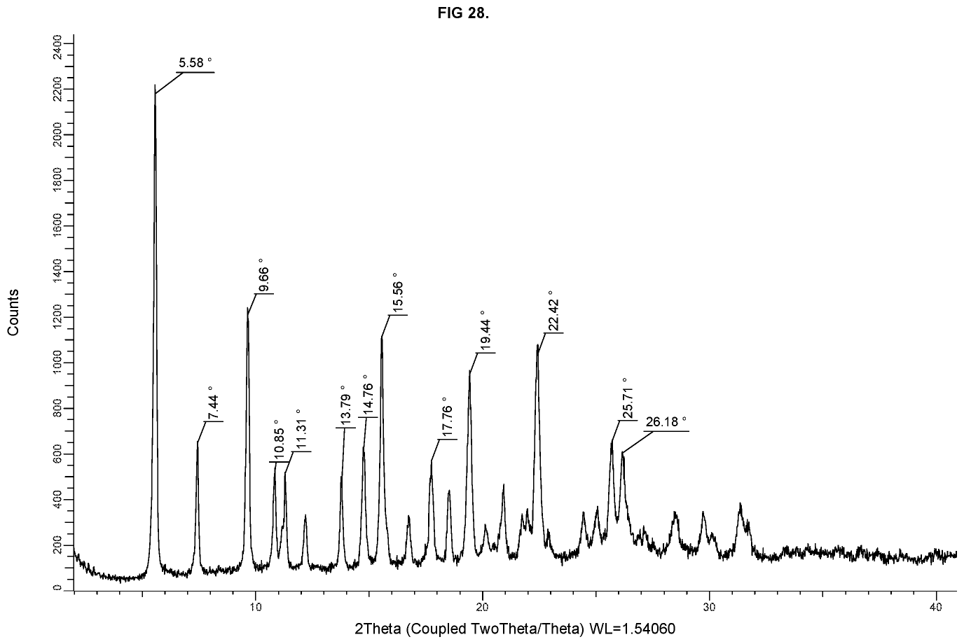

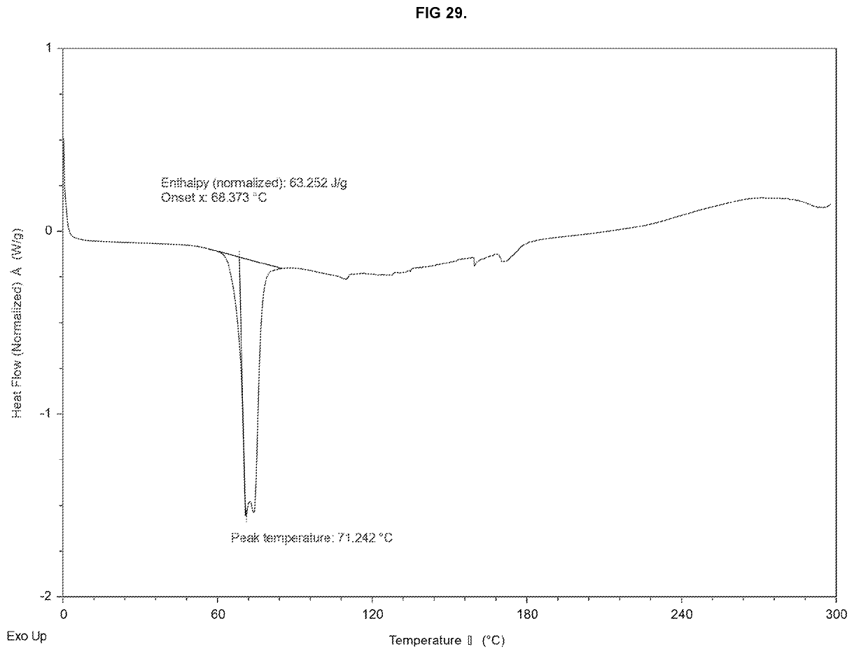

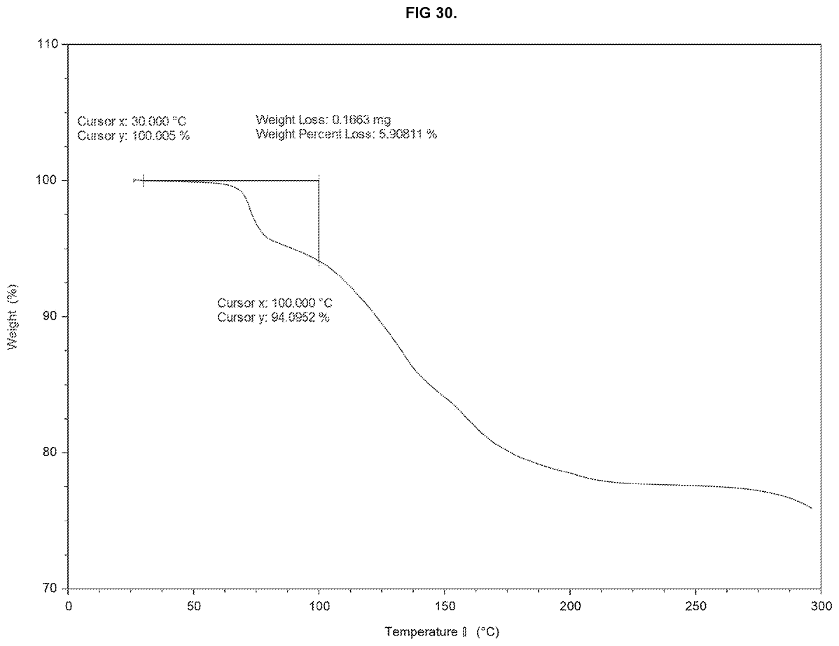

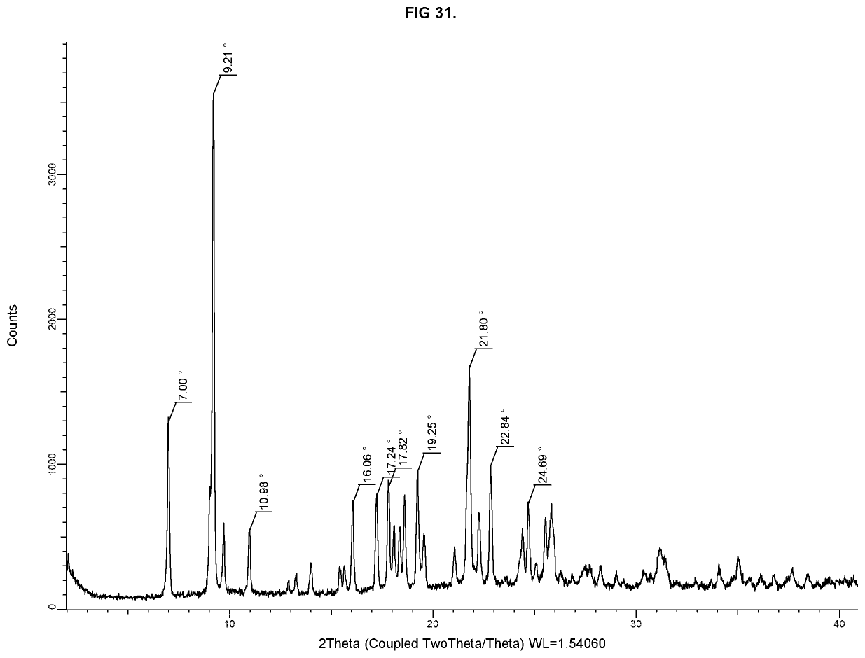

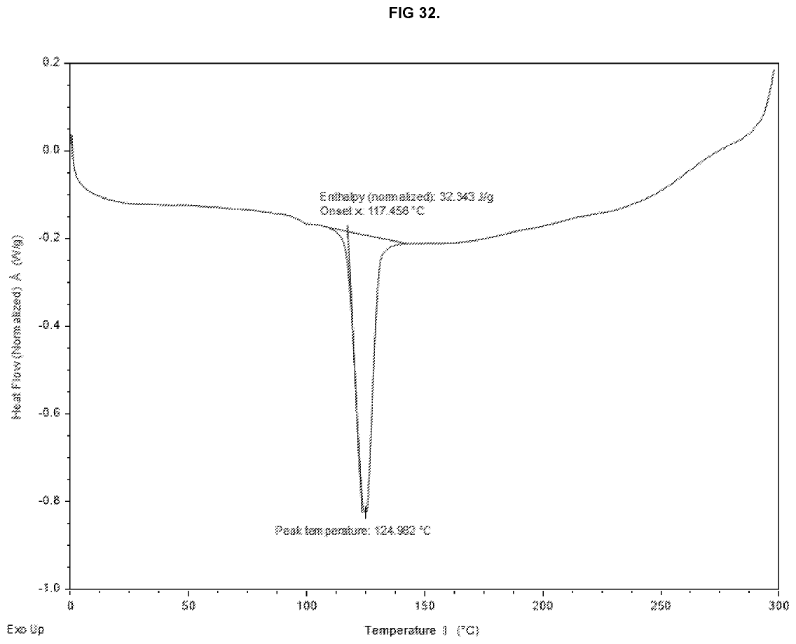

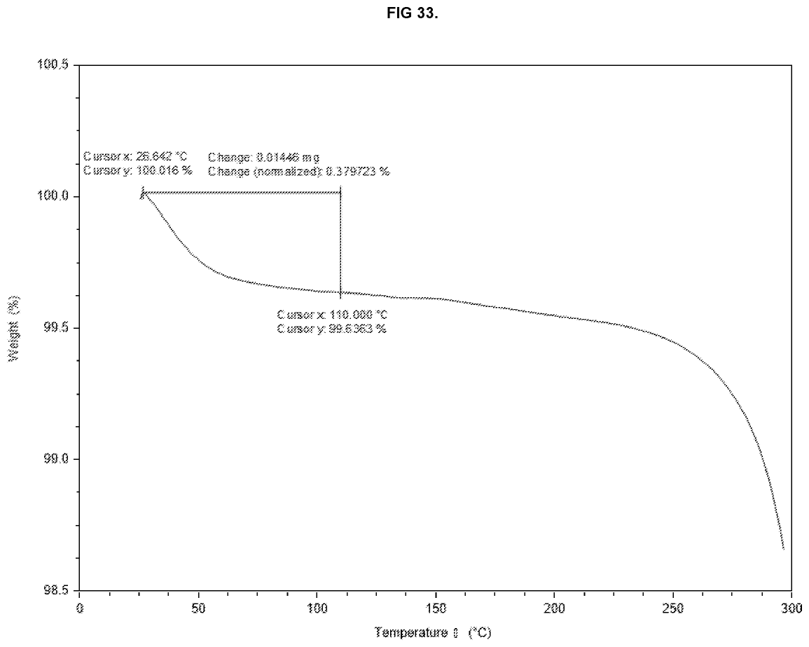

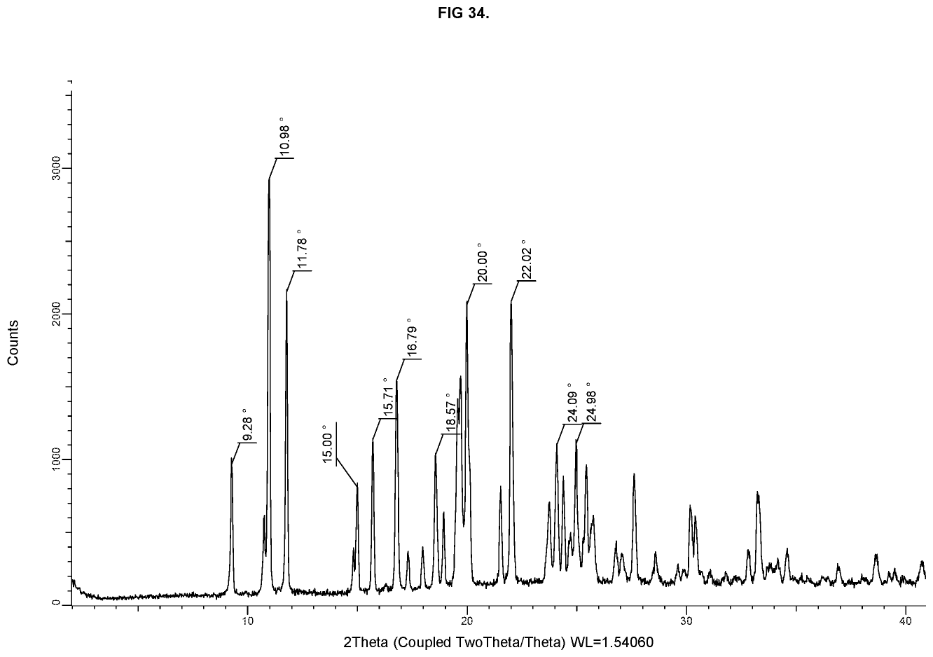

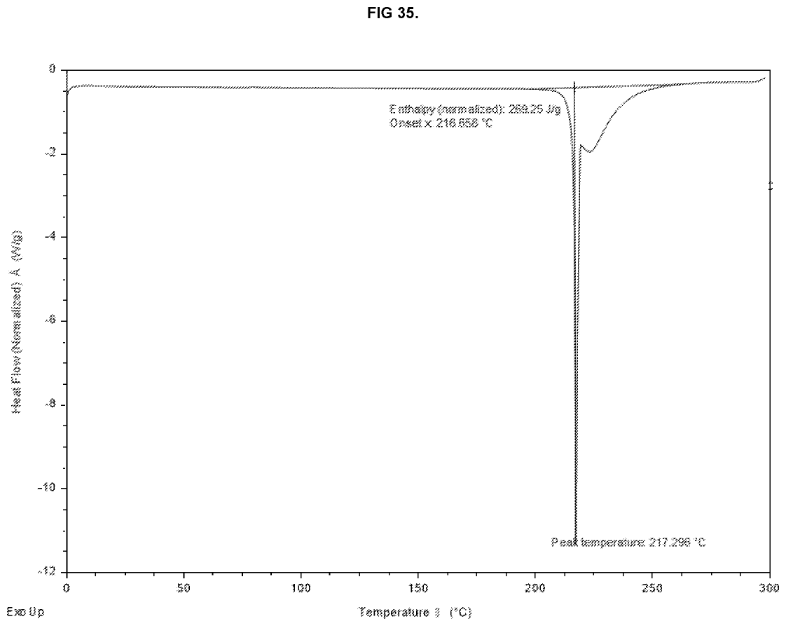

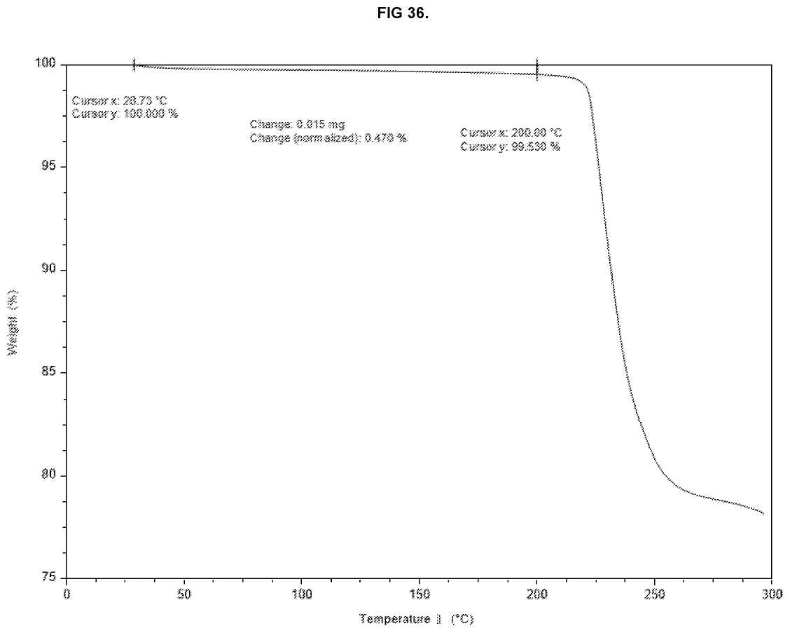

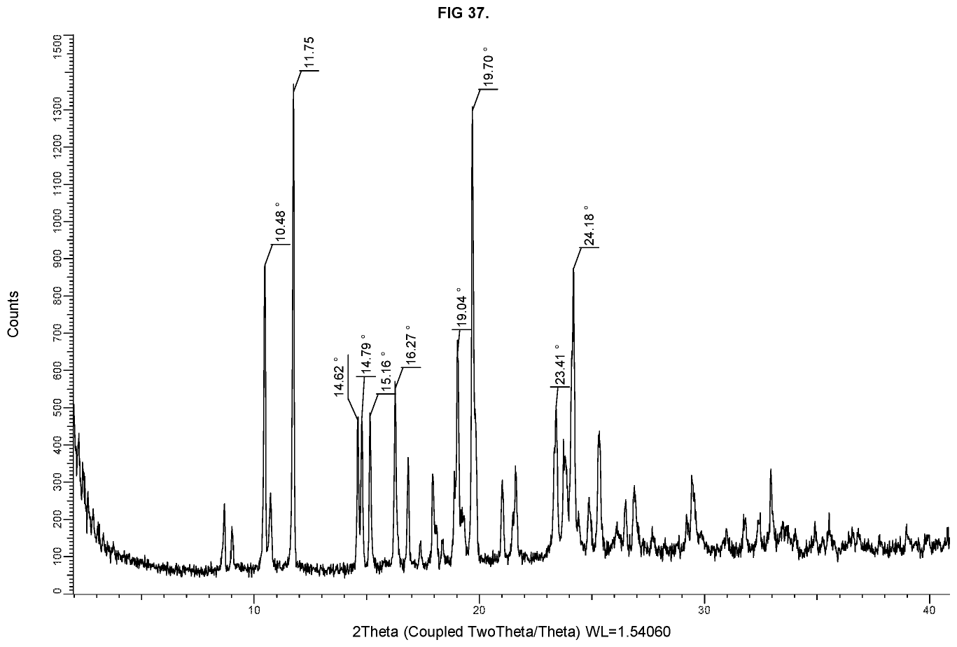

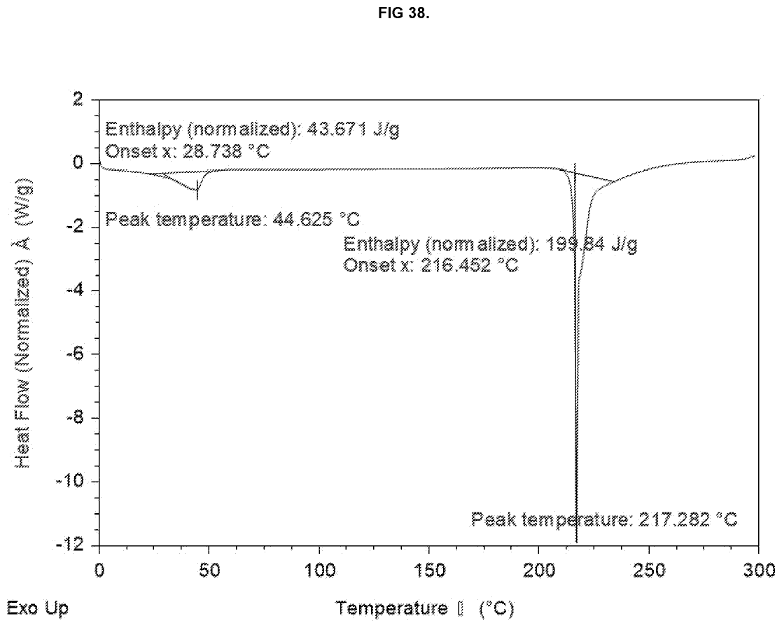

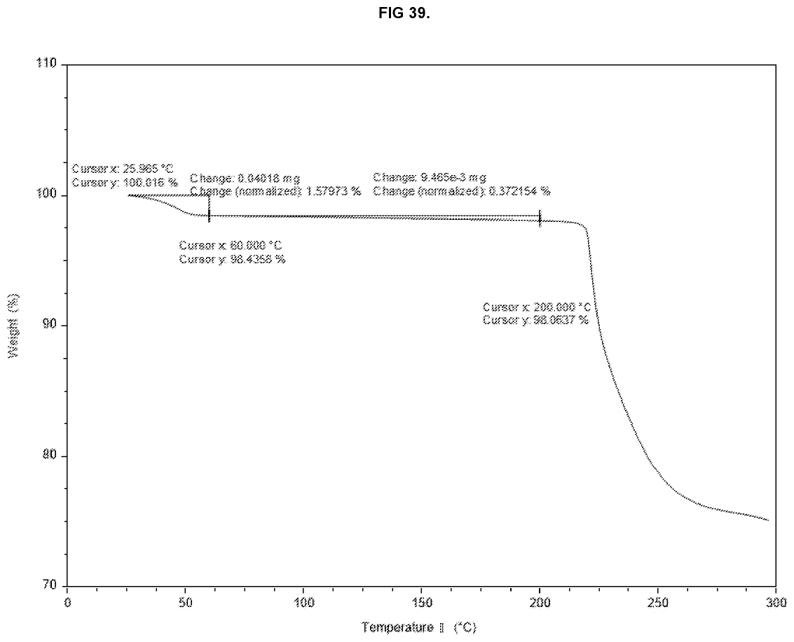

Embodiment 6. The crystalline form according to any one of Embodiments 3 to 5, having a differential scanning calorimetry (DSC) thermogram substantially the same as that shown in shown in FIG. 2.

Embodiment 6a. The crystalline form according to any one of Embodiments 3 to 5, having a melting endotherm with a Tₒₙₛₑₜ of about 200.0° C. when heated from 0 to 300° C. at 10° C. per minute as measured by differential scanning calorimetry using a TA Discovery DSC instrument.

Embodiment 7. The crystalline form according to any one of Embodiments 3 to 6a, having a thermo gravimetric analysis (TGA) diagram substantially the same as that shown in shown in FIG. 3.

Embodiment 7a. The crystalline form according to any one of Embodiments 3 to 6a, having a loss on drying of about 0.45% when heated from 30° C. to 200° C. at a heating rate of 10° C./min, as measured by thermogravimetric analysis using a TA Discovery TGA instrument.

Embodiment 7b. The crystalline form according to any one of Embodiments 3 to 7a, wherein the crystalline form is substantially phase pure.

Embodiment 8. The crystalline form according to Embodiment 1, wherein the Compound B is in the form of a malate salt.

Embodiment 9. The crystalline form according to Embodiment 8, characterized by an X-ray powder diffraction pattern comprising peaks at four or more 2θ values (e.g. 5 or more, e.g. 6 or more, e.g. 7 or more, e.g. 8 or more, e.g. 9 or more, e.g. 10 or more, e.g. 11 or more, e.g. 12 or more, e.g. 13 or more, e.g. all 14 2θ values) selected from the group consisting of:

Angle (2θ)
12.28° ± 0.2°
13.13° ± 0.2°
13.86° ± 0.2°
15.04° ± 0.2°
15.88° ± 0.2°
16.91° ± 0.2°
17.52° ± 0.2°
19.63° ± 0.2°
20.49° ± 0.2°
21.24° ± 0.2°
23.52° ± 0.2°
26.26° ± 0.2°
26.61° ± 0.2°
27.76° ± 0.2°

wherein the temperature is about room temperature and the radiation used has a wavelength of 1.54060 Å, and preferably wherein the X-ray powder diffraction pattern comprises at least the peaks at 13.86°±0.2°, 16.91°±0.2°, 19.63°±0.2° and 23.52°±0.2°.

Embodiment 10. The crystalline form according to Embodiment 8 or Embodiment 9, having an X-ray powder diffraction pattern substantially the same as the X-ray powder diffraction spectrum as shown in FIG. 4, at about room temperature wherein the radiation used has a wavelength of 1.54060 Å.

Embodiment 11. The crystalline form according to any one of Embodiments 8 to 10, having a differential scanning calorimetry (DSC) thermogram substantially the same as that shown in shown in FIG. 5.

Embodiment 11a. The crystalline form according to any one of Embodiments 8 to 10, having a melting endotherm with a Tₒₙₛₑₜ of about 195.4° C. when heated from 0 to 300° C. at 10° C. per minute as measured by differential scanning calorimetry using a TA Discovery DSC instrument.

Embodiment 12. The crystalline form according to any one of Embodiments 8 to 11a, having a thermo gravimetric analysis (TGA) diagram substantially the same as that shown in shown in FIG. 6.

Embodiment 12a. The crystalline form according to any one of Embodiments 8 to 11a, having a loss on drying of about 0.81% when heated from 30° C. to 200° C. at a heating rate of 10° C./min, as measured by thermogravimetric analysis using a TA Discovery TGA instrument.

Embodiment 12b. The crystalline form according to any one of Embodiments 8 to 12a, wherein the crystalline form is substantially phase pure.

Embodiment 13. The crystalline form according to Embodiment 1, wherein the Compound B is in the form of a lactate salt.

Embodiment 14. The crystalline form according to Embodiment 13, characterized by an X-ray powder diffraction pattern comprising peaks at four or more 2θ values (e.g. 5 or more, e.g. 6 or more, e.g. 7 or more, e.g. 8 or more, e.g. 9 or more, e.g. 10 or more, e.g. 11 or more, e.g. 12 or more, e.g. all 13 2θ values) selected from the group consisting of:

Angle (2θ)
10.50° ± 0.2°
13.37° ± 0.2°
14.62° ± 0.2°
16.13° ± 0.2°
16.62° ± 0.2°
18.07° ± 0.2°
21.25° ± 0.2°
22.41° ± 0.2°
22.63° ± 0.2°
22.76° ± 0.2°
23.79° ± 0.2°
26.84° ± 0.2°
30.91° ± 0.2°

wherein the temperature is about room temperature and the radiation used has a wavelength of 1.54060 Å, and preferably wherein the X-ray powder diffraction pattern comprises at least the peaks at 10.50°±0.2°, 13.37°±0.2°, 18.07°±0.2° and 22.41°±0.2°.

Embodiment 15. The crystalline form according to Embodiment 13 or Embodiment 14, having an X-ray powder diffraction pattern substantially the same as the X-ray powder diffraction spectrum as shown in FIG. 7, at about room temperature wherein the radiation used has a wavelength of 1.54060 Å.

Embodiment 16. The crystalline form according to any one of Embodiments 13 to 15, having a differential scanning calorimetry (DSC) thermogram substantially the same as that shown in shown in FIG. 8.

Embodiment 16a. The crystalline form according to any one of Embodiments 13 to 15, having a melting endotherm with a Tₒₙₛₑₜ of about 207.1° C. when heated from 0 to 300° C. at 10° C. per minute as measured by differential scanning calorimetry using a TA Discovery DSC instrument.

Embodiment 17. The crystalline form according to any one of Embodiments 13 to 16a, having a thermo gravimetric analysis (TGA) diagram substantially the same as that shown in shown in FIG. 9.

Embodiment 17a. The crystalline form according to any one of Embodiments 13 to 16a, having a loss on drying of about 0.91% when heated from 30° C. to 200° C. at a heating rate of 10° C./min, as measured by thermogravimetric analysis using a TA Discovery TGA instrument.

Embodiment 17b. The crystalline form according to any one of Embodiments 13 to 17a, wherein the crystalline form is substantially phase pure.

Embodiment 18. The crystalline form according to Embodiment 1, wherein the Compound B is in the form of a benzoate salt.

Embodiment 19. The crystalline form according to Embodiment 18, characterized by an X-ray powder diffraction pattern comprising peaks at four or more 2θ values (e.g. 5 or more, e.g. 6 or more, e.g. 7 or more, e.g. 8 or more, e.g. 9 or more, e.g. 10 or more, e.g. 11 or more, e.g. 12 or more, e.g. all 13 2θ values) selected from the group consisting of:

```
Angle (2θ)
  5.47° ± 0.2°
  8.71° ± 0.2°
10.30° ± 0.2°
10.92° ± 0.2°
12.01° ± 0.2°
12.24° ± 0.2°
13.79° ± 0.2°
14.51° ± 0.2°
20.50° ± 0.2°
21.87° ± 0.2°
23.23° ± 0.2°
29.19° ± 0.2°
31.06° ± 0.2°
```

wherein the temperature is about room temperature and the radiation used has a wavelength of 1.54060 Å, and preferably wherein the X-ray powder diffraction pattern comprises at least the peaks at 5.47°±0.2°, 10.92°±0.2°, 12.24°±0.2° and 21.87°±0.2°.

Embodiment 20. The crystalline form according to Embodiment 18 or Embodiment 19, having an X-ray powder diffraction pattern substantially the same as the X-ray powder diffraction spectrum as shown in FIG. 10, at about room temperature wherein the radiation used has a wavelength of 1.54060 Å.

Embodiment 21. The crystalline form according to any one of Embodiments 18 to 20, having a differential scanning calorimetry (DSC) thermogram substantially the same as that shown in shown in FIG. 11.

Embodiment 21a. The crystalline form according to any one of Embodiments 18 to 20, having a melting endotherm with a Tₒₙₛₑₜ of about 166.8° C. when heated from 0 to 300° C. at 10° C. per minute as measured by differential scanning calorimetry using a TA Discovery DSC instrument.

Embodiment 22. The crystalline form according to any one of Embodiments 18 to 21a, having a thermo gravimetric analysis (TGA) diagram substantially the same as that shown in shown in FIG. 12.

Embodiment 22a. The crystalline form according to any one of Embodiments 18 to 21a, having a loss on drying of about 0.72% when heated from 30° C. to 170° C. at a heating rate of 10° C./min, as measured by thermogravimetric analysis using a TA Discovery TGA instrument.

Embodiment 22b. The crystalline form according to any one of Embodiments 18 to 22a, wherein the crystalline form is substantially phase pure.

Embodiment 23. The crystalline form according to Embodiment 1, wherein the Compound B is in the form of a glutamate salt.

Embodiment 24. The crystalline form according to Embodiment 23, characterized by an X-ray powder diffraction pattern comprising peaks at four or more 2θ values (e.g. 5 or more, e.g. 6 or more, e.g. 7 or more, e.g. 8 or more, e.g. 9 or more, e.g. 10 or more, e.g. all 11 2θ values) selected from the group consisting of:

Angle (2θ)
13.33° ± 0.2°
13.77° ± 0.2°
14.96° ± 0.2°
18.02° ± 0.2°
18.40° ± 0.2°
20.02° ± 0.2°
21.15° ± 0.2°
23.69° ± 0.2°
24.02° ± 0.2°
24.79° ± 0.2°
26.77° ± 0.2°

wherein the temperature is about room temperature and the radiation used has a wavelength of 1.54060 Å, and preferably wherein the X-ray powder diffraction pattern comprises at least the peaks at 13.33°±0.2°, 14.96°±0.2°, 20.02°±0.2° and 26.77°±0.2°.

Embodiment 25. The crystalline form according to Embodiment 23 or Embodiment 24, having an X-ray powder diffraction pattern substantially the same as the X-ray powder diffraction spectrum as shown in FIG. 13, at about room temperature wherein the radiation used has a wavelength of 1.54060 Å.

Embodiment 26. The crystalline form according to any one of Embodiments 23 to 25, having a differential scanning calorimetry (DSC) thermogram substantially the same as that shown in shown in FIG. 14.

Embodiment 26a. The crystalline form according to any one of Embodiments 23 to 25, having melting endotherms with Tₒₙₛₑₜ values of about 26.0° C. and about 158.6° C. when heated from 0 to 300° C. at 10° C. per minute as measured by differential scanning calorimetry using a TA Discovery DSC instrument.

Embodiment 27. The crystalline form according to any one of Embodiments 23 to 26a, having a thermo gravimetric analysis (TGA) diagram substantially the same as that shown in shown in FIG. 15.

Embodiment 27a. The crystalline form according to any one of Embodiments 23 to 26a, having a loss on drying of about 1.45% when heated from 30° C. to 135° C. at a heating rate of 10° C./min, as measured by thermogravimetric analysis using a TA Discovery TGA instrument.

Embodiment 27b. The crystalline form according to any one of Embodiments 23 to 27a, wherein the crystalline form is substantially phase pure.

Embodiment 28. The crystalline form according to Embodiment 1, wherein the Compound B is in the form of a maleate salt.

Embodiment 29. The crystalline form according to Embodiment 28, characterized by an X-ray powder diffraction pattern comprising peaks at four or more 2θ values (e.g. 5 or more, e.g. 6 or more, e.g. 7 or more, e.g. 8 or more, e.g. 9 or more, e.g. 10 or more, e.g. 11 or more, e.g. all 12 2θ values) selected from the group consisting of:

Angle (2θ)
12.80° ± 0.2°
14.22° ± 0.2°
14.57° ± 0.2°
15.35° ± 0.2°
16.11° ± 0.2°
17.71° ± 0.2°
21.38° ± 0.2°
22.92° ± 0.2°
23.88° ± 0.2°
24.39° ± 0.2°
25.51° ± 0.2°
28.61° ± 0.2°

wherein the temperature is about room temperature and the radiation used has a wavelength of 1.54060 Å, and preferably wherein the X-ray powder diffraction pattern comprises at least the peaks at 12.80°±0.2°, 14.57°±0.2°, 16.11°±0.2° and 17.71°±0.2°.

Embodiment 30. The crystalline form according to Embodiment 28 or Embodiment 29, having an X-ray powder diffraction pattern substantially the same as the X-ray powder diffraction spectrum as shown in FIG. 16, at about room temperature wherein the radiation used has a wavelength of 1.54060 Å.

Embodiment 31. The crystalline form according to any one of Embodiments 28 to 30, having a differential scanning calorimetry (DSC) thermogram substantially the same as that shown in shown in FIG. 17.

Embodiment 31a. The crystalline form according to any one of Embodiments 28 to 30, having a melting endotherm with a Tₒₙₛₑₜ of about 204.0° C. when heated from 0 to 300° C. at 10° C. per minute as measured by differential scanning calorimetry using a TA Discovery DSC instrument.

Embodiment 32. The crystalline form according to any one of Embodiments 28 to 31a, having a thermo gravimetric analysis (TGA) diagram substantially the same as that shown in shown in FIG. 18.

Embodiment 32a. The crystalline form according to any one of Embodiments 28 to 31a, having a loss on drying of about 0.60% when heated from 30° C. to 200° C. at a heating rate of 10° C./min, as measured by thermogravimetric analysis using a TA Discovery TGA instrument.

Embodiment 32b. The crystalline form according to any one of Embodiments 28 to 32a, wherein the crystalline form is substantially phase pure.

Embodiment 33. The crystalline form according to Embodiment 1, wherein the Compound B is in the form of a malonate salt.

Embodiment 34. The crystalline form according to Embodiment 33, characterized by an X-ray powder diffraction pattern comprising peaks at four or more 2θ values (e.g. 5 or more, e.g. 6 or more, e.g. 7 or more, e.g. 8 or more, e.g. 9 or more, e.g. 10 or more, e.g. 11 or more, e.g. 12 or more, e.g. all 13 2θ values) selected from the group consisting of:

Angle (2θ)
9.68° ± 0.2°
13.90° ± 0.2°
14.31° ± 0.2°
15.2θ° ± 0.2°
17.28° ± 0.2°
18.29° ± 0.2°
21.2θ° ± 0.2°
21.94° ± 0.2°
22.23° ± 0.2°
23.63° ± 0.2°
24.07° ± 0.2°
28.07° ± 0.2°
29.17° ± 0.2°

wherein the temperature is about room temperature and the radiation used has a wavelength of 1.54060 Å, and preferably wherein the X-ray powder diffraction pattern comprises at least the peaks at 9.68°±0.2°, 13.90°±0.2°, 21.20°±0.2° and 21.94°±0.2°.

Embodiment 35. The crystalline form according to Embodiment 33 or Embodiment 34, having an X-ray powder diffraction pattern substantially the same as the X-ray powder diffraction spectrum as shown in FIG. 19, at about room temperature wherein the radiation used has a wavelength of 1.54060 Å.

Embodiment 36. The crystalline form according to any one of Embodiments 33 to 35, having a differential scanning calorimetry (DSC) thermogram substantially the same as that shown in shown in FIG. 20.

Embodiment 36a. The crystalline form according to any one of Embodiments 33 to 35, having a melting endotherm with a Tₒₙₛₑₜ of about 186.6° C. when heated from 0 to 300° C. at 10° C. per minute as measured by differential scanning calorimetry using a TA Discovery DSC instrument.

Embodiment 37. The crystalline form according to any one of Embodiments 33 to 36a, having a thermo gravimetric analysis (TGA) diagram substantially the same as that shown in shown in FIG. 21.

Embodiment 37a. The crystalline form according to any one of Embodiments 33 to 36a, having a loss on drying of about 0.51% when heated from 30° C. to 182° C. at a heating rate of 10° C./min, as measured by thermogravimetric analysis using a TA Discovery TGA instrument.

Embodiment 37b. The crystalline form according to any one of Embodiments 34 to 37a, wherein the crystalline form is substantially phase pure.

Embodiment 38. The crystalline form according to Embodiment 33, characterized by an X-ray powder diffraction pattern comprising peaks at four or more 2θ values (e.g. 5 or more, e.g. 6 or more, e.g. 7 or more, e.g. 8 or more, e.g. 9 or more, e.g. 10 or more, e.g. 11 or more, e.g. 12 or more, e.g. all 13 2θ values) selected from the group consisting of:

Angle (2θ)
12.25° ± 0.2°
14.01° ± 0.2°
17.23° ± 0.2°
17.59° ± 0.2°
17.91° ± 0.2°
19.28° ± 0.2°
20.55° ± 0.2°
22.53° ± 0.2°
22.89° ± 0.2°
23.42° ± 0.2°
24.08° ± 0.2°
24.59° ± 0.2°
27.45° ± 0.2°

wherein the temperature is about room temperature and the radiation used has a wavelength of 1.54060 Å, and preferably wherein the X-ray powder diffraction pattern comprises at least the peaks at 12.25°±0.2°, 17.91°±0.2°, 19.28°±0.2° and 24.08°±0.2°.

Embodiment 39. The crystalline form according to Embodiment 33 or Embodiment 38, having an X-ray powder diffraction pattern substantially the same as the X-ray powder diffraction spectrum as shown in FIG. 22, at about room temperature wherein the radiation used has a wavelength of 1.54060 Å.

Embodiment 40. The crystalline form according to any one of Embodiments 33, 38 and 39, having a differential scanning calorimetry (DSC) thermogram substantially the same as that shown in shown in FIG. 23.

Embodiment 40a. The crystalline form according to any one of Embodiments 33, 38 and 39, having a melting endotherm with a Tₒₙₛₑₜ of about 122.2° C. when heated from 0 to 300° C. at 10° C. per minute as measured by differential scanning calorimetry using a TA Discovery DSC instrument.

Embodiment 41. The crystalline form according to any one of Embodiments 33 and 38 to 40a, having a thermo gravimetric analysis (TGA) diagram substantially the same as that shown in shown in FIG. 24.

Embodiment 41a. The crystalline form according to any one of Embodiments 33 and 38 to 40a, having a loss on drying of about 26.7% when heated from 30° C. to 200° C. at a heating rate of 10° C./min, as measured by thermogravimetric analysis using a TA Discovery TGA instrument.

Embodiment 41b. The crystalline form according to any one of Embodiments 38 to 41a, wherein the crystalline form is substantially phase pure.

Embodiment 42. The crystalline form according to Embodiment 1, wherein the Compound B is in the form of a mesylate salt.

Embodiment 43. The crystalline form according to Embodiment 42, characterized by an X-ray powder diffraction pattern comprising peaks at four or more 2θ values (e.g. 5 or more, e.g. 6 or more, e.g. 7 or more, e.g. 8 or more, e.g. 9 or more, e.g. 10 or more, e.g. 11 or more, e.g. 12 or more, e.g. all 13 2θ values) selected from the group consisting of:

Angle (2θ)
12.99° ± 0.2°
13.76° ± 0.2°
15.78° ± 0.2°
16.46° ± 0.2°
16.91° ± 0.2°
17.42° ± 0.2°
18.18° ± 0.2°
18.99° ± 0.2°
20.40° ± 0.2°
21.13° ± 0.2°
22.42° ± 0.2°
23.32° ± 0.2°
23.57° ± 0.2°

wherein the temperature is about room temperature and the radiation used has a wavelength of 1.54060 Å, and preferably wherein the X-ray powder diffraction pattern comprises at least the peaks at 13.76°±0.2°, 15.78°±0.2°, 16.46°±0.2° and 18.18°±0.2°.

Embodiment 44. The crystalline form according to Embodiment 42 or Embodiment 43, having an X-ray powder diffraction pattern substantially the same as the X-ray powder diffraction spectrum as shown in FIG. 25, at about room temperature wherein the radiation used has a wavelength of 1.54060 Å.

Embodiment 45. The crystalline form according to any one of Embodiments 42 to 44, having a differential scanning calorimetry (DSC) thermogram substantially the same as that shown in shown in FIG. 26.

Embodiment 45a. The crystalline form according to any one of Embodiments 42 to 44, having melting endotherms with Tₒₙₛₑₜ values of about 22° C. and about 267.9° C. when heated from 0 to 300° C. at 10° C. per minute as measured by differential scanning calorimetry using a TA Discovery DSC instrument.

Embodiment 46. The crystalline form according to any one of Embodiments 42 to 45a, having a thermo gravimetric analysis (TGA) diagram substantially the same as that shown in shown in FIG. 27.

Embodiment 46a. The crystalline form according to any one of Embodiments 42 to 45a, having a loss on drying of about 0.34% when heated from 30° C. to 100° C. at a heating rate of 10° C./min, as measured by thermogravimetric analysis using a TA Discovery TGA instrument.

Embodiment 46b. The crystalline form according to any one of Embodiments 42 to 46a, wherein the crystalline form is substantially phase pure.

Embodiment 47. The crystalline form according to Embodiment 1, wherein the Compound B is in free form.

Embodiment 48. The crystalline form according to Embodiment 47, wherein the Compound B is in the form of Compound B free form 2-methyl-2-butanol solvate

Embodiment 49. The crystalline form according to Embodiment 47 or Embodiment 48, characterized by an X-ray powder diffraction pattern comprising peaks at four or more 2θ values (e.g. 5 or more, e.g. 6 or more, e.g. 7 or more, e.g. 8 or more, e.g. 9 or more, e.g. 10 or more, e.g. 11 or more, e.g. 12 or more, e.g. all 13 2θ values) selected from the group consisting of:

```
Angle (2θ)
  5.58° ± 0.2°
  7.44° ± 0.2°
  9.66° ± 0.2°
10.85° ± 0.2°
11.31° ± 0.2°
13.79° ± 0.2°
14.76° ± 0.2°
15.56° ± 0.2°
17.76° ± 0.2°
19.44º ± 0.2º
22.42° ± 0.2°
25.71° ± 0.2°
26.18° ± 0.2°
```

wherein the temperature is about room temperature and the radiation used has a wavelength of 1.54060 Å, and preferably wherein the X-ray powder diffraction pattern comprises at least the peaks at 5.58°±0.2°, 9.66°±0.2°, 15.56°±0.2° and 19.44°±0.2°.

Embodiment 50. The crystalline form according to any one of Embodiments 47 to 49, having an X-ray powder diffraction pattern substantially the same as the X-ray powder diffraction spectrum as shown in FIG. 28, at about room temperature wherein the radiation used has a wavelength of 1.54060 Å.

Embodiment 51. The crystalline form according to any one of Embodiments 47 to 50, having a differential scanning calorimetry (DSC) thermogram substantially the same as that shown in shown in FIG. 29.

Embodiment 51a. The crystalline form according to any one of Embodiments 47 to 50, having a melting endotherm with a Tₒₙₛₑₜ of about 68° C. when heated from 0 to 300° C. at 10° C. per minute as measured by differential scanning calorimetry using a TA Discovery DSC instrument.

Embodiment 52. The crystalline form according to any one of Embodiments 47 to 51a, having a thermo gravimetric analysis (TGA) diagram substantially the same as that shown in shown in FIG. 30.

Embodiment 52a. The crystalline form according to any one of Embodiments 47 to 51a, having a loss on drying of about 5.91% when heated from 30° C. to 100° C. at a heating rate of 10° C./min, as measured by thermogravimetric analysis using a TA Discovery TGA instrument.

Embodiment 52b. The crystalline form according to any one of Embodiments 47 to 52a, wherein the crystalline form is substantially phase pure.

Embodiment 53. The crystalline form according to Embodiment 2, wherein the Compound A is free form Compound A or a solvate thereof.

Embodiment 54. The crystalline form according to Embodiment 53, characterized by an X-ray powder diffraction pattern comprising four or more 2θ values (e.g. 5 or more, e.g. 6 or more, e.g. 7 or more, e.g. 8 or more, e.g. 9 or more, e.g. all ten 2θ values) selected from the group consisting of:

```
Angle
  7.00° ± 0.2°
  9.21° ± 0.2°
10.98° ± 0.2°
16.06° ± 0.2°
17.24° ± 0.2°
17.82° ± 0.2°
19.25° ± 0.2°
21.80° ± 0.2°
22.84° ± 0.2°
24.69° ± 0.2°
```

wherein the temperature is about room temperature and the radiation used has a wavelength of 1.54060 Å, and preferably wherein the X-ray powder diffraction pattern comprises at least the peaks at 7.00°±0.2°, 9.21°±0.2°, 10.98°±0.2° and 21.80°±0.2°.

Embodiment 55. The crystalline form according to Embodiment 53 or Embodiment 54, having an X-ray powder diffraction pattern substantially the same as the X-ray powder diffraction spectrum as shown in FIG. 31, at about room temperature wherein the radiation used has a wavelength of 1.54060 Å.

Embodiment 56. The crystalline form according to any one of Embodiments 53 to 55, wherein the Compound A is a solvate of free form Compound A.

Embodiment 57. The crystalline form according to any one of Embodiments 53 to 56, having a differential scanning calorimetry (DSC) thermogram substantially the same as that shown in shown in FIG. 32.

Embodiment 57a. The crystalline form according to any one of Embodiments 53 to 56, having a melting endotherm with a Tₒₙₛₑₜ of about 117.5° C. when heated from 0 to 300° C. at 10° C. per minute as measured by differential scanning calorimetry using a TA Discovery DSC instrument.

Embodiment 58. The crystalline form according to any one of Embodiments 53 to 57a, having a thermo gravimetric analysis (TGA) diagram substantially the same as that shown in shown in FIG. 33.

Embodiment 58a. The crystalline form according to any one of Embodiments 53 to 57a, having a loss on drying of about 0.38% when heated from 27° C. to 110° C. at a heating rate of 10° C./min, as measured by thermogravimetric analysis using a TA Discovery TGA instrument.

Embodiment 58b. The crystalline form according to any one of Embodiments 53 to 58a, wherein the crystalline form is substantially phase pure.

Embodiment 59. The crystalline form according to Embodiment 2, wherein the Compound A is in the form of a 4-hydroxybenzoate salt.

Embodiment 60. The crystalline form according to Embodiment 59, characterized by an X-ray powder diffraction pattern comprising four or more 2θ values (e.g. 5 or more, e.g. 6 or more, e.g. 7 or more, e.g. 8 or more, e.g. 9 or more, e.g. 10 or more, e.g. all 11 2θ values) selected from the group consisting of:

Angle
9.28° ± 0.2°
10.98° ± 0.2°
11.78° ± 0.2º
15.00° ± 0.2°
15.71° ± 0.2°
16.79° ± 0.2°
18.57° ± 0.2°
20.00° ± 0.2°
22.02° ± 0.2°
24.09° ± 0.2°
24.98° ± 0.2°

wherein the temperature is about room temperature and the radiation used has a wavelength of 1.54060 Å, and preferably wherein the X-ray powder diffraction pattern comprises at least the peaks at 10.98°±0.2°, 11.78°±0.2°, 16.79°±0.2° and 20.00°±0.2°.

Embodiment 61. The crystalline form according to Embodiment 59 or Embodiment 60, having an X-ray powder diffraction pattern substantially the same as the X-ray powder diffraction spectrum as shown in FIG. 34, at about room temperature wherein the radiation used has a wavelength of 1.54060 Å.

Embodiment 62. The crystalline form according to any one of Embodiments 59 to 61, having a differential scanning calorimetry (DSC) thermogram substantially the same as that shown in shown in FIG. 35.

Embodiment 62a. The crystalline form according to any one of Embodiments 59 to 61, having a melting endotherm with a Tₒₙₛₑₜ of about 216.7° C. when heated from 0 to 300° C. at 10° C. per minute as measured by differential scanning calorimetry using a TA Discovery DSC instrument.

Embodiment 63. The crystalline form according to any one of Embodiments 59 to 62a, having a thermo gravimetric analysis (TGA) diagram substantially the same as that shown in shown in FIG. 36.

Embodiment 63a. The crystalline form according to any one of Embodiments 59 to 62a, having a loss on drying of about 0.46% when heated from 27° C. to 110° C. at a heating rate of 10° C./min, as measured by thermogravimetric analysis using a TA Discovery TGA instrument.

Embodiment 63b. The crystalline form according to any one of Embodiments 59 to 63a, wherein the crystalline form is substantially phase pure.

Embodiment 64. The crystalline form according to Embodiment 2, wherein the Compound A is in the form of a 3,4-dihydroxybenzoate salt.

Embodiment 65. The crystalline form according to Embodiment 64, characterized by an X-ray powder diffraction pattern comprising four or more 2θ values (e.g. 5 or more, e.g. 6 or more, e.g. 7 or more, e.g. 8 or more, e.g. 9 or more, e.g. all 10 2θ values) selected from the group consisting of:

Angle
10.48° ± 0.2°
11.75° ± 0.2°
14.62° ± 0.2°
14.79° ± 0.2°
15.16° ± 0.2°
16.27° ± 0.2°
19.04° ± 0.2°
19.70° ± 0.2°
23.41° ± 0.2°
24.18° ± 0.2°

wherein the temperature is about room temperature and the radiation used has a wavelength of 1.54060 Å, and preferably wherein the X-ray powder diffraction pattern comprises at least the peaks at 10.48°±0.2°, about 11.75°±0.2°, 16.27°±0.2° and 19.70°±0.2°.

Embodiment 66. The crystalline form according to Embodiment 64 or Embodiment 65, having an X-ray powder diffraction pattern substantially the same as the X-ray powder diffraction spectrum as shown in FIG. 37, at about room temperature wherein the radiation used has a wavelength of 1.54060 Å.

Embodiment 67. The crystalline form according to any one of Embodiments 64 to 66, having a differential scanning calorimetry (DSC) thermogram substantially the same as that shown in shown in FIG. 38.

Embodiment 67a. The crystalline form according to any one of Embodiments 64 to 66, having melting endotherms with Tₒₙₛₑₜ values of about 29° C. and about 216.5° C. when heated from 0 to 300° C. at 10° C. per minute as measured by differential scanning calorimetry using a TA Discovery DSC instrument.

Embodiment 68. The crystalline form according to any one of Embodiments 64 to 67a, having a thermo gravimetric analysis (TGA) diagram substantially the same as that shown in shown in FIG. 39.

Embodiment 68a. The crystalline form according to any one of Embodiments 64 to 67a, having a loss on drying of about 1.63% when heated from 26° C. to 80° C. at a heating rate of 10° C./min, as measured by thermogravimetric analysis using a TA Discovery TGA instrument.

Embodiment 68b. The crystalline form according to any one of Embodiments 64 to 68a, wherein the crystalline form is substantially phase pure.

Embodiment 69. A pharmaceutical composition comprising the crystalline form of any one of the preceding Embodiments and a pharmaceutically acceptable carrier.

Embodiment 70. The crystalline form according to any one of Embodiments 1 to 68a, or the pharmaceutical composition according to Embodiment 69 for use as a medicament.

Embodiment 71. A combination comprising a crystalline form of any of Embodiments 1 to 68a and one or more therapeutically active agents.

Embodiment 72. The crystalline form of any one of Embodiments 1 to 68a or the pharmaceutical composition according to Embodiment 69 for use in treating a disease or condition mediated by YAP overexpression and/or YAP amplification and/or YAP/TAZ-TEAD interaction; or for use in treating a cancer or tumor harboring (i) one or more YAP/TAZ fusions; (ii) one or more NF2/LATS1/LATS2 truncating mutations or deletions; or (iii) one or more functional YAP/TAZ fusions.

Embodiment 73. A method of treating a disease or condition mediated by YAP overexpression and/or YAP amplification and/or YAP/TAZ-TEAD interaction, or a method of treating a cancer or tumor harboring (i) one or more YAP/TAZ fusions; (ii) one or more NF2/LATS1/LATS2 truncating mutations or deletions; or (iii) one or more functional YAP/TAZ fusions; said method comprising administering to a subject in need thereof, a therapeutically effective amount of a crystalline form according to any one of Embodiments 1 to 68a; or a pharmaceutical composition according to Embodiment 69; or a combination according to Embodiment 71.

Embodiment 74. The crystalline form according to any one of Embodiments 1 to 68a, or the pharmaceutical composition according to Embodiment 69 for use in the treatment of cancer, preferably wherein the cancer is selected from a cancer or tumor which is selected from mesothelioma (including pleural mesothelioma, malignant pleural mesothelioma, peritoneal mesothelioma, pericardial mesothelioma and mesothelioma of the tunica vaginalis), carcinoma (including cervical squamous cell carcinoma, endometrial carcinoma, esophageal squamous cell carcinoma, esophageal adenocarcinoma, urothelial carcinoma of the bladder and squamous cell carcinoma of the skin), poroma (benign poroma), porocarcinoma (including malignant porocarcinoma), supratentorial ependymoma (including childhood supratentorial ependymoma), epithelioid hemangioendothelioma (EHE), ependymal tumor, a solid tumor, breast cancer (including triple negative breast cancer), lung cancer (including non-small cell lung cancer), ovarian cancer, colorectal cancer (including colorectal carcinoma), melanoma, pancreatic cancer (including pancreatic adenocarcinoma), prostate cancer, gastric cancer, esophageal cancer, liver cancer (including hepatocellular carcinoma, cholangiocarcinoma and hepatoblastoma), neuroblastoma, Schwannoma, kidney cancer, sarcoma (including rhabdomyosarcoma, embryonic rhabdomyosarcoma (ERMS), osteosarcoma, undifferentiated pleomorphic sarcomas (UPS), Kaposi's sarcoma, soft-tissue sarcoma and rare soft-tissue sarcoma), bone cancer, brain cancer, medulloblastoma, glioma, meningioma, and head and neck cancer (including head and neck squamous cell carcinoma).

Embodiment 75. The method according to Embodiment 73, wherein the cancer, tumor, disease or condition is selected from mesothelioma (including pleural mesothelioma, malignant pleural mesothelioma, peritoneal mesothelioma, pericardial mesothelioma and mesothelioma of the tunica vaginalis), carcinoma (including cervical squamous cell carcinoma, endometrial carcinoma, esophageal squamous cell carcinoma, esophageal adenocarcinoma, urothelial carcinoma of the bladder and squamous cell carcinoma of the skin), poroma (benign poroma), porocarcinoma (including malignant porocarcinoma), supratentorial ependymoma (including childhood supratentorial ependymoma), epithelioid hemangioendothelioma (EHE), ependymal tumor, a solid tumor, breast cancer (including triple negative breast cancer), lung cancer (including non-small cell lung cancer), ovarian cancer, colorectal cancer (including colorectal carcinoma), melanoma, pancreatic cancer (including pancreatic adenocarcinoma), prostate cancer, gastric cancer, esophageal cancer, liver cancer (including hepatocellular carcinoma, cholangiocarcinoma and hepatoblastoma), neuroblastoma, Schwannoma, kidney cancer, sarcoma (including rhabdomyosarcoma, embryonic rhabdomyosarcoma (ERMS), osteosarcoma, undifferentiated pleomorphic sarcomas (UPS), Kaposi's sarcoma, soft-tissue sarcoma and rare soft-tissue sarcoma), bone cancer, brain cancer, medulloblastoma, glioma, meningioma, and head and neck cancer (including head and 1 neck squamous cell carcinoma).

Embodiment 76. The method according to Embodiment 73, wherein the disease or condition is selected from mesothelioma (including pleural mesothelioma, malignant pleural mesothelioma, peritoneal mesothelioma, pericardial mesothelioma and mesothelioma of the tunica vaginalis), and solid tumors with NF2/LATS1/LATS2 mutations.

Embodiment 77. Compound B in the form of a succinate salt.

Embodiment 78. Compound B in the form of a malate salt.

Embodiment 79. Compound B in the form of a lactate salt.

Embodiment 80. Compound B in the form of a benzoate salt.

Embodiment 81. Compound B in the form of a glutamate salt.

Embodiment 82. Compound B in the form of a maleate salt.

Embodiment 83. Compound B in the form of a malonate salt.

Embodiment 84. Compound B in the form of a mesylate salt.

Embodiment 85. Compound B in the form of Compound B free form, e.g. Compound B free form 2-methyl-2-butanol solvate.

Embodiment 86. Compound A in the form of Compound A free form.

Embodiment 87. Compound A in the form of a 4-hydroxybenzoate salt.

Embodiment 88. Compound A in the form of a 3,4-dihydroxybenzoate salt.

### Definitions

As used herein “polymorph” or “crystalline modification(s)” or “crystalline form” refers to crystalline forms having the same chemical composition but different spatial arrangements of the molecules, atoms, and/or ions forming the crystal.

As used herein “solvate” refers to a crystalline form of a molecule, atom, and/or ions that further comprises molecules of a solvent or solvents incorporated into the crystalline lattice structure. The solvent molecules in the solvate may be present in a regular arrangement and/or a non-ordered arrangement. The solvate may comprise either a stoichiometric or nonstoichiometric amount of the solvent molecules. For example, a solvate with a nonstoichiometric amount of solvent molecules may result from partial loss of solvent from the solvate. Solvates may occur as dimers or oligomers comprising more than one molecule or Compound ABC within the crystalline lattice structure. The solvent may be water, in which case the solvent may be referred to as a hydrate.

As used herein, the term “free form” of a given compound refers to a solid state form where the only component present which is solid at ambient conditions (e.g. 20° C., 1 atm) is the said compound. Thus, as used herein, the term “free form” encompasses both unsolvated/unhydrated forms, and solvated/hydrated forms, but excludes salts and co-crystals where the coformer is solid at ambient conditions.

As used herein, the terms “salt” or “salts” refers to an acid addition or base addition salt of a compound of the present invention. “Salts” include in particular “pharmaceutical acceptable salts”. The term “pharmaceutically acceptable salts” refers to salts that retain the biological effectiveness and properties of the compounds of this invention and, which typically are not biologically or otherwise undesirable. In many cases, the compounds of the present invention are capable of forming acid and/or base salts by virtue of the presence of amino and/or carboxyl groups or groups similar thereto. When both a basic group and an acid group are present in the same molecule, the compounds of the present invention may also form internal salts, e.g., zwitterionic molecules.

Pharmaceutically acceptable acid addition salts can be formed with inorganic acids and organic acids.

Inorganic acids from which salts can be derived include, for example, hydrochloric acid, hydrobromic acid, sulfuric acid, nitric acid, phosphoric acid, and the like.

Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like.

Pharmaceutically acceptable base addition salts can be formed with inorganic and organic bases.

Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

As used herein “amorphous” refers to a solid form of a molecule, atom, and/or ions that is not crystalline. An amorphous solid does not display a definitive X-ray diffraction pattern.

As used herein, the term “substantially phase pure” with reference to a particular polymorphic form means that the polymorphic form includes less than 10%, preferably less than 5%, more preferably less than 3%, most preferably less than 1% by weight of any other phases (polymorphs) of the same compound.

The term “essentially the same” with reference to X-ray diffraction peak positions means that typical peak position and intensity variability are taken into account. For example, one skilled in the art will appreciate that the peak positions (28) will show some inter-apparatus variability, typically as much as 0.2°. Further, one skilled in the art will appreciate that relative peak intensities will show inter-apparatus variability as well as variability due to degree of crystallinity, preferred orientation, prepared sample surface, and other factors known to those skilled in the art, and should be taken as qualitative measure only. The person skilled in the art of X-ray powder diffraction is readily able to determine whether a given sample comes from the same polymorph as a reference sample.

As used herein, the terms “about” and “substantially” indicate with respect to features such as endotherms, endothermic peak, exotherms, baseline shifts, etc., that their values can vary. With reference to X-ray diffraction peak positions, “about” or “substantially” means that typical peak position and intensity variability are taken into account. For example, one skilled in the art will appreciate that the peak positions (2θ) will show some inter-apparatus variability, typically as much as 0.2°. Occasionally, the variability could be higher than 0.2° depending on apparatus calibration differences. Further, one skilled in the art will appreciate that relative peak intensities will show inter-apparatus variability as well as variability due to degree of crystallinity, preferred orientation, prepared sample surface, and other factors known to those skilled in the art, and should be taken as qualitative measure only. For DSC, variation in the temperatures observed will depend upon the rate of temperature change as well as sample preparation technique and the particular instrument employed. Thus, the endotherm/melting point values reported herein relating to DSC/TGA thermograms can vary ±5° C. (and still be considered to be characteristic of the particular crystalline form described herein). When used in the context of other features, such as, for example, percent by weight (% by weight), reaction temperatures, the term “about” indicates a variance of ±5%.

The term “a therapeutically effective amount” of a crystalline form of the present invention refers to an amount of the crystalline form of the present invention that will elicit the biological or medical response of a subject, for example, reduction or inhibition of an enzyme or a protein activity, or ameliorate symptoms, alleviate conditions, slow or delay disease progression, or prevent a disease, etc. In one non-limiting embodiment, the term “a therapeutically effective amount” refers to the amount of the compound of the present invention that, when administered to a subject, is effective to (1) at least partially alleviating, inhibiting, preventing and/or ameliorating a condition, or a disorder or a disease associated with (i) hyperactivation of the YAP/TAZ-TEAD complex (ii) mediated by YAP overexpression and/or YAP amplification, or (iii) associated with YAP activity, or (iv) characterized by activity (normal or abnormal) of YAP; or (2) reducing or inhibiting the interaction of YAP and/or TAZ with TEAD. In another non-limiting embodiment, the term “a therapeutically effective amount” refers to the amount of the crystalline form of the present invention that, when administered to a cell, or a tissue, or a non-cellular biological material, or a medium, is effective to at least partially reducing or inhibiting the interaction of YAP and/or TAZ with TEAD.

As used herein, the term “a,” “an,” “the” and similar terms used in the context of the present invention (especially in the context of the claims) are to be construed to cover both the singular and plural unless otherwise indicated herein or clearly contradicted by the context.

All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed.

As used herein, the term “inhibit”, “inhibition” or “inhibiting” refers to the reduction or suppression of a given condition, symptom, or disorder, or disease, or a significant decrease in the baseline activity of a biological activity or process.

As used herein, the terms “treat,” “treating,” or “treatment” of any disease or disorder refers in one embodiment, to ameliorating the disease or disorder (i.e., slowing or arresting or reducing the development of the disease or at least one of the clinical symptoms thereof). In another embodiment, “treat,” “treating,” or “treatment” refers to alleviating or ameliorating at least one physical parameter including those which may not be discernible by the patient. In yet another embodiment, “treat,” “treating,” or “treatment” refers to modulating the disease or disorder, either physically, (e.g., stabilization of a discernible symptom), physiologically, (e.g., stabilization of a physical parameter), or both. In one embodiment, “treat” or “treating” refers to delaying the progression of the disease or disorder.

As used herein, the term “prevent”, “preventing” or “prevention” of any disease or disorder refers to the prophylactic treatment of the disease or disorder; or delaying the onset of the disease or disorder.

As used herein, the term “subject” refers to an animal. Preferably, the animal is a mammal. A subject refers to for example, primates (e.g. humans), cows, sheep, goats, horses, dogs, cats, rabbits, rats, mice, fish, birds and the like. In a preferred embodiment, the subject is a human.

As used herein, a subject is “in need of” or “in need thereof” a treatment if such subject would benefit biologically, medically or in quality of life from such treatment.

The term “comprising” encompasses “including” as well as “consisting”; e.g., a composition comprising X may consist exclusively of X or may include additional, e.g. X and Y.

The crystalline form of the present invention may be administered either simultaneously with, or before or after, one or more other therapeutic agent. The crystalline form of the present invention may be administered separately, by the same or different route of administration, or together in the same pharmaceutical composition as the other agents. A therapeutic agent is, for example, a chemical compound, peptide, antibody, antibody fragment or nucleic acid, which is therapeutically active or enhances the therapeutic activity when administered to a patient in combination with a compound of the present invention.

In the combination therapies of the invention, the crystalline form of the present invention and the other therapeutic agent may be manufactured and/or formulated by the same or different manufacturers. Moreover, the crystalline form of the present invention and the other therapeutic may be brought together into a combination therapy: (i) prior to release of the combination product to physicians (e.g. in the case of a kit comprising the crystalline form of the present invention and the other therapeutic agent); (ii) by the physician themselves (or under the guidance of the physician) shortly before administration; (iii) in the patient themselves, e.g. during sequential administration of the crystalline form of the present invention and the other therapeutic agent.

Synthesis of the compounds of the invention was originally described in PCT/IB2021/052136 (WO2021/186324), the contents of which are incorporated by reference.

### Solid State Chemistry of Compound B

### XRPD Method

X-ray powder diffraction (XRPD) patterns were obtained using a Bruker Advance D8 in reflection geometry. Powders were analyzed using a zero background Si flat sample holder. The radiation used was Cu Kα (A=1.5418 Å). Patterns were measured between 2° and 40° 2theta.
Sample amount: 5-10 mg
Sample holder: zero background Si flat sample holder

### XRPD Parameter

| | |
|---|---|
| Instrument | Bruker D8 Advance |
| Detector | LYNXEYE (1D mode), open angle: 2.948°, |
| | scan mode: continuos scan |
| Radiation | Cu Kα (0.15418 nm) |
| Monochromator | Nickel filter |
| X-ray generator power | 40 KV, 40 mA |
| Goniometer radius | 280 mm |
| Step size | 0.0164°(2-theta value) |
| Time per step | 0.3 second per step |
| Scan range | 2° to 40° (2-theta value) |
| Scan time | About 768 seconds |
| Slits | Primary: fixed illuminated sample size 10 mm; |
| | secondary: open angle 2.2°, axial soller: 2.5° |

The most characteristic peaks in XRPD of each form are highlighted in red and marked as A, B, C, D

### 1. Basic Characterization of Compound B Crystalline Forms and Preparation Examples

### 1) Characterization of Compound B Succinate Salt

a. XRPD Pattern of Compound B Succinate Salt

(See FIG. 1 for XRPD pattern, strongest peaks are shown below)

| Index | Angle | d Value | Rel. Intensity | Intensity |
|---|---|---|---|---|
| 1 | 11.93 | 7.41 | 9.9% | |
| 2-A | 13.26 | 6.67 | 100.0% | Strong |
| 3 | 13.77 | 6.42 | 26.2% | |
| 4 | 15.12 | 5.86 | 11.1% | |
| 5-B | 16.99 | 5.22 | 32.0% | Medium |
| 6 | 17.39 | 5.09 | 12.2% | |
| 7-C | 19.92 | 4.45 | 91.5% | Strong |
| 8 | 20.73 | 4.28 | 10.6% | |
| 9 | 21.06 | 4.21 | 19.0% | |
| 10 | 23.45 | 3.79 | 10.5% | |
| 11 | 24.11 | 3.69 | 22.7% | |
| 12 | 26.37 | 3.38 | 9.9% | |
| 13-D | 26.66 | 3.34 | 68.9% | Strong |

b. Unit Cell of Compound B Succinate Salt

A preliminary experimental crystal structure (BDI35A) of Compound B Modification A was determined at 100 K. The structure of BDI35A contains 1 API cation and 1 hydrogen succinate anion in the asymmetric unit (Z′=1) with a space group of P2₁. The crystal structure information is listed in the Table below.

| Parameter | Value |
|---|---|
| Crystal system | Monoclinic |
| Space group | P2₁ |
| Cell lengths (Å) | a 12.7585, b 8.298, c 14.715 |
| Cell angles (°) | α 90.0, β 114.804, γ 90.0 |
| Cell volume (Å³) | 1414.16 |

c. DSC Thermogram of Compound B Succinate Salt

(See FIG. 2)

d. TGA Thermogram of Compound B Succinate Salt

(See FIG. 3)

e. Preparation Method of Compound B Succinate Salt

Example 1: About 70 mg Compound B and 19 mg succinic acid was weighed in a vial, then 1 mL ethyl acetate was added. The sample was stirred at 50° C. for around 2-4 hours, then stirred at r.t. overnight. The solid was collected by centrifuge filtration and dried at 40° C. for 2 h.

Example 2: About 70 mg Compound B and 19 mg succinic acid was weighed in a vial, then 1 mL THF was added. The sample was stirred at 50° C. for around 2-4 hours, then stirred at r.t. overnight. The solid was collected by centrifuge filtration and dried at 40° C. for 2h.

Example 3: About 70 mg Compound B and 19 mg succinic acid was weighed in a vial, then 1 mL acetonitrile/water (95/5, v/v) was added. The sample was stirred at 50° C. for around 2-4 hours, then stirred at r.t. overnight. The solid was collected by centrifuge filtration and dried at 40° C. for 2h.

Example 4: About 20 mg Compound B and 5.5 mg succinic acid was weighed in a vial, then 0.15 mL methanol was added. Then 1.35 mL IPA was slowly added to the solution. The sample was shaken at r.t. overnight. The solid was collected by centrifuge filtration.

Example 5: About 20 mg Compound B and 5.5 mg succinic acid was weighed in a vial, then 0.15 mL methanol was added. Then 1.35 mL MIBK was slowly added to the solution. The sample was shaken at r.t. overnight. The solid was collected by centrifuge filtration.

Example 6: About 20 mg Compound B and 5.5 mg succinic acid was weighed in a vial, then 0.15 mL methanol was added. Then 1.35 mL EA was slowly added to the solution. The sample was shaken at r.t. overnight. The solid was collected by centrifuge filtration.

Example 7: About 20 mg Compound B and 5.5 mg succinic acid was weighed in a vial, then 0.15 mL methanol was added. Then 1.35 mL MTBE was slowly added to the solution. The sample was shaken at r.t. overnight. The solid was collected by centrifuge filtration.

Example 8: Weigh 1.0 g free form (Compound B) and 275 mg succinic acid into an reactor, add 5.5 ml methanol to dissolve the solid at RT. Add 50 ml IPA as antisolvent with 300 rpm stirring in 1h at RT, then stir overnight at RT. Collect the solid by vacuum filtration and dry under 50° C. overnight. 0.95 g succinate salt was obtained with a yield of 75%.

Example 9: Weigh 8.0 g free form (Compound B) and 2.2 g succinic acid into an reactor, then add 62.7 mL of MeOH/IPA (9/2,v/v) and stir with a peddle under 300 rpm at 55° C. to get a clear solution. Add 10.6 mL IPA, and then add 50 mg seeds (0.5% w/w). Cool to 45° C. in 30 mins. Then add 184 mL IPA in around 5h. Cool to 5° C. in the rate of 0.2K/min and stir at 5° C. overnight. Collect the solid by filtration under vacuum and dry at 50 C for 2h. 8.72 g succinate salt was obtained with a yield of 86%.

### 2) Characterization of Compound B L-Malate Salt

a. XRPD Pattern of Compound B L-Malate Salt

(See FIG. 4 for XRPD pattern, strongest peaks are shown below)

| Index | Angle | d Value | Rel. Intensity | Intensity |
|---|---|---|---|---|
| 1 | 12.28° | 7.20 Å | 20.3% | |
| 2 | 13.13° | 6.74 Å | 33.6% | |
| 3-A | 13.86° | 6.38 Å | 59.6% | Medium |
| 4 | 15.04° | 5.89 Å | 36.6% | |
| 5 | 15.88° | 5.58 Å | 34.6% | |
| 6-B | 16.91° | 5.24 Å | 100.0% | Strong |
| 7 | 17.52° | 5.06 Å | 31.4% | |
| 8-C | 19.63° | 4.52 Å | 43.9% | Medium |
| 9 | 20.49° | 4.33 Å | 37.4% | |
| 10 | 21.24° | 4.18 Å | 39.6% | |
| 11-D | 23.52° | 3.78 Å | 86.4% | Strong |
| 12 | 26.26° | 3.39 Å | 35.6% | |
| 13 | 26.61° | 3.35 Å | 33.0% | |
| 14 | 27.76° | 3.21 Å | 21.0% | |

b. DSC Thermogram of Compound B L-Malate Salt

(See FIG. 5)

c. TGA Thermogram of Compound B L-Malate Salt

(See FIG. 6)

d. Preparation Method for Compound B L-Malate Salt

Example 1: About 70 mg Compound B and 22 mg L-malic acid was weighed in a vial, then 1 mL ethyl acetate was added. The sample was stirred at 50° C. for around 2-4 hours, then stirred at r.t. overnight. The solid was collected by centrifuge filtration and dried at 40° C. for 2h.

Example 2: About 20 mg Compound B and 6.2 mg L-malic acid was weighed in a vial, then 0.15 mL methanol was added. Then 1.35 mL MIBK was slowly added to the solution. The sample was shaken at r.t. for 3 days. The solid was collected by centrifuge filtration.

Example 3: About 20 mg Compound B and 6.2 mg L-malic acid was weighed in a vial, then 0.15 mL methanol was added. Then 1.35 mL EA was slowly added to the solution. The sample was shaken at r.t. for 3 days. The solid was collected by centrifuge filtration.

Example 4: About 20 mg Compound B and 6.2 mg L-malic acid was weighed in a vial, then 0.15 mL methanol was added. Then 1.35 mL MTBE was slowly added to the solution. The sample was shaken at r.t. overnight. The solid was collected by centrifuge filtration.

Example 5: Weigh 2.1 g free form (Compound B) and dissolve in 21 mL EA solvent at 60° C., cloudy solution appeared. Weigh 663.6 mg L-malic acid and dissolve in 21 mL EA solvent at 60° C., clear solution was observed. The dissolved L-malic acid solution was dropped to free from solution at 60° C. with an adding rate of 0.2 mL/min by using a peristaltic pump. Gel was appeared in the system after adding L-malic acid solution. Seeds were added to the mixture and the following temperature profile was applied. Cool down from 60° C. to 20° C. in 4h (i.e. a cooling rate of 0.17° C./min), heat from 20° C. to 50° C. in 3h, cool down from 50° C. to 0° C. in 5 h, the temperature profile was repeated and finally kept at 0° C. overnight with a stirring rate of 300 rpm. The precipitated solids were filtrated and dried at 40° C. for 3 h, 2.35 g material was obtained (yield=87.2%).

Example 6: Weigh 1.0 g free form (Compound B) and 283 mg L-malic acid in a vial, add 3.5 mL MEOH/EA (4/6, v/v) and stir at 25 under 300 rpm to get a clear solution. Add 1.05 mL EA to the clear solution. Add 5.1 mg (0.5%) seeds and stir for another 10 min. Then add 9.45 mL EA in the rate of 0.1 mL/min. Cool to 5° C. in the rate of 0.2K/min and stir at 5° C. overnight. Collect the solid by vacuum filtration and dry at 40° C. under vacuum for 2h. 1.07 g L-malate salt was obtained with a yield of 83.4%.

### 3) Characterization of Compound B L-Lactate Salt

a. XRPD Pattern of Compound B L-Lactate Salt

(See FIG. 7 for XRPD pattern, strongest peaks are shown below)

| Index | Angle | d Value | Rel. Intensity | Intensity |
|---|---|---|---|---|
| 1-A | 10.50° | 8.42 Å | 53.4% | Medium |
| 2-B | 13.37° | 6.61 Å | 100.0% | Strong |
| 3 | 14.62° | 6.06 Å | 23.5% | |
| 4 | 16.13° | 5.49 Å | 19.3% | |
| 5 | 16.62º | 5.33 Å | 29.6% | |
| 6-C | 18.07° | 4.90 Å | 52.3% | Medium |
| 7 | 21.25° | 4.18 Å | 44.0% | |
| 8-D | 22.41º | 3.96 Å | 52.1% | Medium |
| 9 | 22.63° | 3.93 Å | 28.6% | |
| 10 | 22.76° | 3.90 Å | 27.5% | |
| 11 | 23.79° | 3.74 Å | 25.5% | |
| 12 | 26.84° | 3.32 Å | 21.2% | |
| 13 | 30.91° | 2.89 Å | 22.1% | |

b. DSC Thermogram of Compound B L-Lactate Salt

(See FIG. 8)

c. TGA Thermogram of Compound B L-Lactate Salt

(See FIG. 9)

d. Preparation Method for Compound B L-Lactate Salt

Example 1: About 70 mg Compound B and 15 mg L-lactic acid was weighed in a vial, then 1 mL ethyl acetate was added. The sample was stirred at 50° C. for around 2-4 hours, then stirred at r.t. overnight. The solid was collected by centrifuge filtration and dried at 40° C. for 2h.

Example 2: About 70 mg Compound B and 15 mg L-lactic acid was weighed in a vial, then 1 mL THF was added. The sample was stirred at 50° C. for around 2-4 hours, then stirred at r.t. overnight. The solid was collected by centrifuge filtration and dried at 40° C. for 2h.

Example 3: About 70 mg Compound B and 15 mg L-lactic acid was weighed in a vial, then 1 mL acetonitrile/water (95/5, v/v) was added. The sample was stirred at 50° C. for around 2-4 hours, then stirred at r.t. overnight. A clear solution was obtained and evaporated to dryness at r.t. The solid was collected and dried at 40° C. for 2h.

Example 4: Weigh 2.1 g free form (Compound B) and dissolve in 21 mL EA solvent at 60° C., cloudy solution appeared. Weigh 445.8 mg L-lactic acid and dissolve in 21 mL EA solvent at 60° C., clear solution was observed. Free form solution was dropped to the dissolved L-lactic acid solution at 60° C. A suspension appeared after adding L-lactic acid solution. Seeds were added to the mixture and the following temperature profile was applied. Cool down from 60° C. to 20° C. in 4h (i.e. a cooling rate of 0.17° C./min), heat from 20° C. to 50° C. in 3h, cool down from 50° C. to 0° C. in 5 h, the temperature profile was repeated and finally kept at 0° C. overnight with a stirring rate of 300 rpm. The precipitated solids were filtrated and dried at 40° C. for 3 h, 2.15 g material was obtained (yield=86%).

### 4) Characterization of Compound B Benzoate Salt

a. XRPD Pattern of Benzoate Salt

(See FIG. 10 for XRPD pattern, strongest peaks are shown below)

| Index | Angle | d Value | Rel. Intensity | Intensity |
|---|---|---|---|---|
| 1-A | 5.47° | 16.15 Å | 26.5% | Medium |
| 2 | 8.71° | 10.15 Å | 15.2% | |
| 3 | 10.30° | 8.58 Å | 24.0% | |
| 4-B | 10.92° | 8.09 Å | 100.0% | Strong |
| 5 | 12.01° | 7.36 Å | 17.3% | |
| 6-C | 12.24° | 7.23 Å | 50.8% | Medium |
| 7 | 13.79º | 6.41 Å | 14.2% | |
| 8 | 14.51° | 6.10 Å | 19.0% | |
| 9 | 20.50° | 4.33 Å | 25.0% | |
| 10-D | 21.87° | 4.06 Å | 59.6% | Medium |
| 11 | 23.23° | 3.83 Å | 29.6% | |
| 12 | 29.19° | 3.06 Å | 19.8% | |
| 13 | 31.06° | 2.88 Å | 16.1% | |

b. DSC Thermogram of Compound B Benzoate Salt

(See FIG. 11)

c. TGA Thermogram of Compound B Benzoate Salt

(See FIG. 12)

d. Preparation Method of Compound B Benzoate Salt

Example 1: About 70 mg Compound B and 20 mg benzoic acid was weighed in a vial, then 1 mL ethyl acetate was added. The sample was stirred at 50° C. for around 2-4 hours, then stirred at r.t. overnight. The solid was collected by centrifuge filtration and dried at 40° C. for 2h.

Example 2: About 70 mg Compound B and 20 mg benzoic acid was weighed in a vial, then 1 mL acetonitrile/water (95/5, v/v) was added. The sample was stirred at 50° C. for around 2-4 hours, then stirred at r.t. overnight. The solid was collected by centrifuge filtration and dried at 40° C. for 2h.

### 5) Characterization of Compound B Glutamate Salt

a. XRPD Pattern of Compound B Glutamate Salt

(See FIG. 13 for XRPD pattern, strongest peaks are shown below)

| Index | Angle | d Value | Rel. Intensity | Intensity |
|---|---|---|---|---|
| 1-A | 13.33° | 6.64 Å | 69.2% | Strong |
| 2 | 13.77° | 6.42 Å | 20.2% | |
| 3-B | 14.96º | 5.92 Å | 81.2% | Strong |
| 4 | 18.02° | 4.92 Å | 38.4% | |
| 5 | 18.40° | 4.82 Å | 20.0% | |
| 6-C | 20.02° | 4.43 Å | 100.0% | Strong |
| 7 | 21.15° | 4.20 Å | 45.3% | |
| 8 | 23.69° | 3.75 Å | 30.2% | |
| 9 | 24.02° | 3.70 Å | 70.3% | Strong |
| 10 | 24.79° | 3.59 Å | 54.2% | |
| 11-D | 26.77° | 3.33 Å | 79.8% | Strong |

b. DSC Thermogram of Compound B Glutamate Salt

(See FIG. 14)

c. TGA Thermogram of Compound B Glutamate Salt

(See FIG. 15)

d. Preparation Method of Compound B Glutamate Salt

Example 1: About 70 mg Compound B and 22 mg glutamic acid was weighed in a vial, then 1 mL ethyl acetate was added. The sample was stirred at 50° C. for around 2-4 hours, then stirred at r.t. overnight. The solid was collected by centrifuge filtration and dried at 40° C. for 2h.

Example 2: About 70 mg Compound B and 22 mg glutamic acid was weighed in a vial, then 1 mL acetonitrile/water (95/5, v/v) was added. The sample was stirred at 50° C. for around 2-4 hours, then stirred at r.t. overnight. A clear solution was obtained and evaporated to dryness at r.t. The solid was collected and dried at 40° C. for 2h.

### 6) Characterization of Compound B Maleate Salt

a. XRPD Pattern of Compound B Maleate Salt

(See FIG. 16 for XRPD pattern, strongest peaks are shown below)

| Index | Angle | d Value | Rel. Intensity | Intensity |
|---|---|---|---|---|
| 1-A | 12.80° | 6.91 Å | 62.4% | Strong |
| 2 | 14.22° | 6.22 Å | 30.5% | |
| 3-B | 14.57° | 6.07 Å | 100.0% | Strong |
| 4 | 15.35° | 5.77 Å | 20.9% | |
| 5-C | 16.11° | 5.50 Å | 50.4% | Medium |
| 6-D | 17.71° | 5.00 Å | 64.5% | Strong |
| 7 | 21.38° | 4.15 Å | 38.4% | |
| 8 | 22.92° | 3.88 Å | 21.5% | |
| 9 | 23.88° | 3.72 Å | 28.4% | |
| 10 | 24.39° | 3.65 Å | 37.1% | |
| 11 | 25.51° | 3.49 Å | 25.5% | |
| 12 | 28.61° | 3.12 Å | 39.7% | |

b. DSC Thermogram of Compound B Maleate

(See FIG. 17)

c. TGA Thermogram of Compound B Maleate

(See FIG. 18)

d. Preparation Method of Compound B Maleate Salt

Example 1: About 70 mg Compound B and 19 mg maleic acid was weighed in a vial, then 1 mL EA was added. The sample was stirred at 50° C. for around 2-4 hours, then stirred at r.t. overnight. The solid was collected by centrifuge filtration and dried at 40° C. for 2h.

Example 2: About 70 mg Compound B and 19 mg maleic acid was weighed in a vial, then 1 mL THF was added. The sample was stirred at 50° C. for around 2-4 hours, then stirred at r.t. overnight. The solid was collected by centrifuge filtration and dried at 40° C. for 2h.

Example 3: About 70 mg Compound B and 19 mg maleic acid was weighed in a vial, then 1 mL acetonitrile/water (95/5, v/v) was added. The sample was stirred at 50° C. for around 2-4 hours, then stirred at r.t. overnight. A clear solution was obtained and evaporated to dryness at r.t. The solid was collected and dried at 40° C. for 2h.

### 7) Characterization of Compound B Malonate Salt Type I

a. XRPD Pattern of Malonate Salt Type I

(See FIG. 19 for XRPD pattern, strongest peaks are shown below)

| Index | Angle | d Value | Rel. Intensity | Intensity |
|---|---|---|---|---|
| 1-A | 9.68° | 9.13 Å | 11.1% | Medium |
| 2-B | 13.90° | 6.37 Å | 100.0% | Strong |
| 3 | 14.31° | 6.18 Å | 7.9% | |
| 4 | 15.20° | 5.83 Å | 8.3% | |
| 5 | 17.28° | 5.13 Å | 17.4% | |
| 6 | 18.29° | 4.85 Å | 8.2% | |
| 7-C | 21.20° | 4.19 Å | 45.8% | Strong |
| 8-D | 21.94° | 4.05 Å | 30.7% | Medium |
| 9 | 22.23° | 4.00 Å | 8.4% | |
| 10 | 23.63° | 3.76 Å | 7.0% | |
| 11 | 24.07° | 3.70 Å | 12.3% | |
| 12 | 28.07° | 3.18 Å | 9.8% | |
| 13 | 29.17° | 3.06 Å | 6.6% | |

b. DSC Thermogram of Compound B Malonate Salt Type I

(See FIG. 20)

c. TGA Thermogram of Compound B Malonate Salt Type I

(See FIG. 21)

d. Preparation Method of Compound B Malonate Salt Type 1

Example 1: About 70 mg Compound B and 17 mg malonic acid was weighed in a vial, then 1 mL EA was added. The sample was stirred at 50° C. for around 2-4 hours, then stirred at r.t. overnight. The solid was collected by centrifuge filtration and dried at 40° C. for 2h.

### 8) Characterization of Compound B Malonate Salt Type II

a. XRPD Pattern of Malonate Salt Type II

(See FIG. 22 for XRPD pattern, strongest peaks are shown below)

| Index | Angle | d Value | Rel. Intensity | Intensity |
|---|---|---|---|---|
| 1-A | 12.25° | 7.22 Å | 100.0% | Strong |
| 2 | 14.01° | 6.32 Å | 8.2% | |
| 3 | 17.23° | 5.14 Å | 7.8% | |
| 4 | 17.59° | 5.04 Å | 9.0% | |
| 5-B | 17.91° | 4.95 Å | 20.3% | Medium |
| 6-C | 19.28° | 4.60 Å | 32.7% | Medium |
| 7 | 20.55° | 4.32 Å | 6.5% | |
| 8 | 22.53° | 3.94 Å | 10.6% | |
| 9 | 22.89° | 3.88 Å | 16.8% | |
| 10 | 23.42° | 3.80 Å | 9.3% | |
| 11-D | 24.08° | 3.69 Å | 32.4% | Medium |
| 12 | 24.59° | 3.62 Å | 24.1% | |
| 13 | 27.45° | 3.25 Å | 9.4% | |

b. DSC thermogram of Compound B malonate salt type II

(See FIG. 23)

c. TGA Thermogram of Compound B Malonate Salt Type II

(See FIG. 24)

d. Preparation Method of Compound B Malonate Salt Type II

Example 1: About 70 mg Compound B and 17 mg malonic acid was weighed in a vial, then 1 mL THF was added. The sample was stirred at 50° C. for around 2-4 hours, then stirred at r.t. overnight. The solid was collected by centrifuge filtration and dried at 40° C. for 2h.

### 9) Characterization of Compound B Mesylate Salt

a. XRPD Pattern of Compound B Mesylate Salt

(See FIG. 25 for XRPD pattern, strongest peaks are shown below)

| Index | Angle | d Value | Rel. Intensity | Intensity |
|---|---|---|---|---|
| 1 | 12.99° | 6.81 Å | 84.4% | |
| 2-A | 13.76° | 6.43 Å | 66.6% | Strong |
| 3-B | 15.78º | 5.61 Å | 100.0% | Strong |
| 4-C | 16.46 | 5.38 Å | 69.8% | Strong |
| 5 | 16.91° | 5.24 Å | 77.9% | |
| 6 | 17.42° | 5.09 Å | 90.8% | |
| 7-D | 18.18° | 4.88 Å | 66.4% | Strong |
| 8 | 18.99° | 4.67 Å | 45.8% | |
| 9 | 20.40° | 4.35 Å | 63.9% | |
| 10 | 21.13° | 4.20 Å | 50.9% | |
| 11 | 22.42° | 3.96 Å | 52.2% | |
| 12 | 23.32° | 3.81 Å | 89.6% | |
| 13 | 23.57° | 3.77 Å | 47.7% | |

b. DSC Thermogram of Compound B Mesylate Salt

(See FIG. 26)

c. TGA Thermogram of Compound B Mesylate Salt

(See FIG. 27)

d. Preparation Method of Compound B Mesylate Salt

Example 1: About 70 mg Compound B was weighed in a vial, and 1 mL EA was added. Then, about 11 μL methanesulfonic acid was added. The sample was stirred at 50° C. for around 2-4 hours, then stirred at r.t. overnight. The solid was collected by centrifuge filtration and dried at 40° C. for 2h.

Example 2: About 70 mg Compound B was weighed in a vial, and 1 mL THF was added. Then, about 11 μL methanesulfonic acid was added. The sample was stirred at 50° C. for around 2-4 hours, then stirred at r.t. overnight. The solid was collected by centrifuge filtration and dried at 40° C. for 2h.

Example 3: About 70 mg Compound B was weighed in a vial, and 1 mL acetonitrile/water (95/5, v/v) was added. Then, about 11 μL methanesulfonic acid was added. The sample was stirred at 50° C. for around 2-4 hours, then stirred at r.t. overnight. A clear solution was obtained and evaporated to dryness at r.t. The solid was collected and dried at 40° C. for 2 h.

### 10) Characterization of Compound B Free Form 2-Methyl-2-Butanol Solvate

a. XRPD Pattern of Compound B Free Form 2-Methyl-2-Butanol Solvate

(See FIG. 28 for XRPD pattern, strongest peaks are shown below)

| Index | Angle | d Value | Rel. Intensity | Intensity |
|---|---|---|---|---|
| 1-A | 5.58° | 15.83 Å | 100.0% | Strong |
| 2 | 7.44° | 11.87 Å | 27.1% | |
| 3-B | 9.66° | 9.15 Å | 52.6% | Medium |
| 4 | 10.85° | 8.15 Å | 19.7% | |
| 5 | 11.31° | 7.82 Å | 19.3% | |
| 6 | 13.79° | 6.42 Å | 17.3% | |
| 7 | 14.76° | 6.00 Å | 23.9% | |
| 8-C | 15.56° | 5.69 Å | 47.2% | Medium |
| 9 | 17.76° | 4.99 Å | 20.9% | |
| 10-D | 19.44º | 4.56 Å | 38.0% | |
| 11 | 22.42° | 3.96 Å | 41.7% | Medium |
| 12 | 25.71° | 3.46 Å | 22.8% | |
| 13 | 26.18° | 3.40 Å | 20.5% | |

b. DSC Thermogram of Compound B Free Form 2-Methyl-2-Butanol Solvate

(See FIG. 29)

c. TGA Thermogram of Compound B Free Form 2-Methyl-2-Butanol Solvate

(See FIG. 30)

d. Preparation Method for Compound B 2-Methyl-2-Butanol Solvate

Example 1: Weigh 40 mg of free form (Compound B) in a vial, add 0.2 mL 2-methyl-2-butanol and stirred at 25 C for 4 weeks. The solid was collected by centrifuge filtration and dried at r.t.

Example 2: 1 g of Compound B was weighed and added to 5 mL 2M2B, seeds were added and slurry at RT for 5 hours. Solids were filtered and washed with 5 mL 2M2B, then dried at ambient condition overnight, followed by drying at 40 degree C. for 0.5h.

### Compound B Stability Data

| | Physical Forms | | | | | | | |
|---|---|---|---|---|---|---|---|---|
| | Compound | | | | | | | |
| | Free form, | | Succinate | | L-malate | | L-lactate | |
| | amorphous | | salt | | salt | | salt | |
| | Initial purity (%) | | | | | | | |
| | 99.89 | | 99.92 | | 99.84 | | 99.83 | |
| Test Conditions | DP[%] | CL | DP[%] | CL | DP[%] | CL | DP[%] | CL |

Solid state, 2 weeks 80° C./11% RH

| | | | | | | | | |
|---|---|---|---|---|---|---|---|---|
| Bulk (HPLC) | 13.00 | C | 0.08 | A | 0.46 | A | 0.31 | A |
| Bulk (XRPD) | gel-like | C | No change | A | No change | A | No change | A |

Solid state, 2 weeks 80° C./75% RH

| | | | | | | | | |
|---|---|---|---|---|---|---|---|---|
| Bulk (HPLC) | 14.88 | C | 0.17 | A | 2.84 | A | 2.11 | A |
| Bulk (XRPD) | gel-like | C | No change | A | No change | A | No change | A |

Solid state, 2 weeks 50° C./75% RH

| | | | | | | | | |
|---|---|---|---|---|---|---|---|---|
| Bulk (HPLC) | 4.84 | A | 0.09 | A | 0.33 | A | 0.33 | A |
| Bulk (XRPD) | gel-like | A | No change | A | No change | A | No change | A |

Xenon light (1200 kLuxh, 25° C.)

| | | | | | | | | |
|---|---|---|---|---|---|---|---|---|
| Bulk clear quartz | 2.74 | C | 2.38 | B | 1.53 | B | 0.38 | B |
| vial (HPLC) | | | | | | | | |
| Bulk clear quartz | No change | C | No change | B | No change | B | No change | B |
| vial (XRPD) | | | | | | | | |
| Bulk amber vial | 0.19 | A | 0.13 | A | 0.16 | A | 0.17 | A |
| (HPLC) | | | | | | | | |
| Bulk amber vial | No change | A | No change | A | No change | A | No change | A |
| (XRPD) | | | | | | | | |

Excipient compatibility, 2 weeks 50° C./closed

| | | | | | | | | |
|---|---|---|---|---|---|---|---|---|
| 1% in HPMC powder | 2.02 | A | 0.17 | A | 0.36 | A | 0.33 | A |
| 1% in HGC powder | 1.87 | A | 0.15 | A | 0.24 | A | 0.22 | A |

Excipient compatibility, 2 weeks 50° C./75% RH

| | | | | | | | | |
|---|---|---|---|---|---|---|---|---|
| 1% in HPMC powder | 4.33 | A | 0.99 | A | 2.65 | A | 0.42 | A |
| 1% in HGC powder | 2.50 | A | 0.25 | A | 1.32 | A | 0.44 | A |

Degradation products (DP) and Color (CL)
↓ Suspension
* Clear solution after stress test
− Test not performed
A No change
B Slight discoloration
C Medium discoloration
D Strong discoloration
DPs are analyzed by HPLC. They are calculated as area-% products or against external standards.
HPMC = Hydroxypropyl Methylcellulose
HGC = Hard gelatin capsule

### Compound B Solubility Data

| Physical Forms | | | |
|---|---|---|---|
| Compound | | | |
| Free form, | Succinate | L-malate | L-lactate |
| amorphous | salt | salt | salt |

Solubility (in mg/mL after equilibration at 25° C. for 24 h, target concentration 2 mg/mL, final pH
and XRPD of solid residues, use 0.45 μm PVDF (polyvinylidene difluoride) membrane for
separation if needed, Limit of Quantification = 0.0003 mg/mL)

| | Sol. | | Sol. | | Sol. | | Sol. | |
|---|---|---|---|---|---|---|---|---|
| Parameter | [pH] | XRPD | [pH] | XRPD | [pH] | XRPD | [pH] | XRPD |
| pH 1.2, 100 mM HCl | >2 | — | >2 | — | >2 | — | >2 | — |
| | [1.16] | | [1.17] | | [1.12] | | [1.12] | |
| pH 3.0, 50 mM | >2 | — | >2 | — | >2 | — | >2 | — |
| citrate buffer | [3.08] | | [3.09] | | [3.04] | | [3.06] | |
| pH 4.7, 50 mM | >2 | — | >2 | — | >2 | — | >2 | — |
| acetate buffer | [4.84] | | [4.70] | | [4.62] | | [4.70] | |
| pH 6.8, 50 mM | >2 | — | >2 | — | >2 | — | >2 | — |
| phosphate buffer | [6.90] | | [6.64] | | [6.59] | | [6.79] | |
| pH 6.8, 50 mM | >2 | — | >2 | — | >2 | — | >2 | — |
| phosphate buffer | [6.72] | | [6.43] | | [6.43] | | [6.58] | |
| with 50 mM Na | | | | | | | | |
| taurocholate | | | | | | | | |
| Water | 0.07 | No | >2 | — | >2 | — | >2 | — |
| | [8.78] | change | [4.97] | | [4.34] | | [7.03] | |
| Blank FaSSIF | 1.49 | No | >2 | — | >2 | — | >2 | — |
| | [7.36] | change | [6.06] | | [6.04] | | [6.53] | |
| SGF pH 2.0 | >2 | — | >2 | — | >2 | — | >2 | — |
| | [2.22] | | [2.20] | | [2.17] | | [2.24] | |
| FaSSIF V2 pH 6.5 | 1.15 | No | >2 | — | >2 | — | >2 | — |
| | [7.19] | change | [6.06] | | [5.95] | | [6.48] | |
| FeSSIF V2 pH 5.8 | >2 | — | >2 | — | >2 | — | >2 | — |
| | [6.01] | | [5.72] | | [5.74] | | [5.79] | |

FaSSIF = Fasted state simulated intestinal fluid
FeSSIF = Fed state simulated intestinal fluid

### Other Properties of Compound B

| Parameter | Free form, | Physical form of Compound B | | |
|---|---|---|---|---|
| Compound B | amorphous | Succinate salt | L-malate salt | L-lactate salt |
| Thermal properties | | | | |
| As is | | | | |
| Melting onset/glass | Tg = 82.9 | 200.3 | 195.5 | 207.1 |
| transition [° C.] | | | | |
| XRPD | Amorphous | High | High | High |
| | | crystallinity | crystallinity | crystallinity |
| After heating and cooling | | | | |
| DSC, glass | N.A. | Tg = 115.3 | Tg = 126.6 | Tg = 96.4 |
| transition [° C.] | | | | |
| ΔCₚ [J/(K · g]) | N.A. | 0.38 | 0.41 | 0.44 |
| Hygroscopicity | | | | |
| As is | | | | |
| Loss on drying by | 1.30%@233° C. | 0.56%@205° C. | 0.81%@200° C. | 0.92%@200° C. |
| TGA | | | | |
| After 1 day at 92% RH | | | | |
| Loss on drying by | 2.14%@233° C. | 0.67%@205° C. | 0.83%@200° C. | 1.18%@200° C. |
| TGA | | | | |
| XRPD | — | — | — | No change |
| After 1 day at 80% RH | | | | |
| Loss on drying by | | | | 1.08%@200° C. |
| TGA | — | — | — | |
| XRPD | — | — | — | No change |

—Test not performed

### Brief Summary of the Selection of Succinate Salt

Succinate salt was selected due to its advantage in counter ion, high crystallinity, bulk stability, hygroscopicity, morphology, as well as process feasibility.

### Solid State Chemistry of Compound A

### 1. XRPD Method

X-ray powder diffraction (XRPD) patterns were obtained using a Bruker Advance D8 in reflection geometry. Powders were analyzed using a zero background Si flat sample holder. The radiation was Cu Kα (λ=1.5418 Å). Patterns were measured between 2° and 40° 2theta.
Sample amount: 5-10 mg
Sample holder: zero background Si flat sample holder

### XRPD Parameter

| | |
|---|---|
| Instrument | Bruker D8 Advance |
| Detector | LYNXEYE (1D mode), open angle: 2.948°, |
| | scan mode: continuos scan |
| Radiation | CuKα (0.15418 nm) |
| Monochromator | Nickel filter |
| X-ray generator power | 40 kV, 40 mA |
| Goniometer radius | 280 mm |
| Step size | 0.0164°(2-theta value) |
| Time per step | 0.3 second per step |
| Scan range | 2° to 40° (2-theta value) |
| Scan time | About 768 seconds |
| Slits | Primary: fixed illuminated sample size 10 mm; |
| | secondary: open angle 2.2°, axial soller: 2.5° |

The most characteristic peaks in XRPD of each form are highlighted in red and marked as A, B, C, D.

### 2. Basic Characterization of Compound A Crystalline Forms and Preparation Examples

### 1) Characterization of Compound A Modification A

a. XRPD Pattern of Compound A Modification A

(See FIG. 31 for XRPD pattern, strongest peaks are shown below)

| Index | Angle | d Value | Rel. Intensity | Intensity |
|---|---|---|---|---|
| 1-A | 7.00° | 12.61901 Å | 35.1% | Medium |
| 2-B | 9.21° | 9.59895 Å | 100.0% | Strong |
| 3-C | 10.98° | 8.05391 Å | 12.5% | Medium |
| 4 | 16.06° | 5.51436 Å | 17.6% | |
| 5 | 17.24° | 5.13948 Å | 18.5% | |
| 6 | 17.82° | 4.97295 Å | 20.0% | |
| 7 | 19.25° | 4.60670 Å | 22.9% | |
| 8-D | 21.80° | 4.07353 Å | 43.3% | Medium |
| 9 | 22.84° | 3.88960 Å | 23.7% | |
| 10 | 24.69° | 3.60279 Å | 15.7% | |

b. DSC Thermogram of Compound A Modification A

(See FIG. 32)

c. TGA Thermogram of Compound A Modification A

(See FIG. 33)

d. Preparation Method of Compound A Modification A

Example 1: About 53 mg of Compound A (amorphous) was weighed into a vial, then 0.4 mL of acetone was added and mixed with 450 rpm at room temperature for 1 h. Then the solid was filtrated and dried at 40 degree C. for 2 hours under vacuum

Example 2: About 53 mg of Compound A (amorphous) was weighed into a vial, then 0.4 mL of acetonitrile was added and mixed with 450 rpm at room temperature for 1 h. Then the solid was filtrated and dried at 40 degree C. for 2 hours under vacuum

Example 3: About 3 g of Compound A amorphous free form was added into 200 mL ACN/water=1/1 at 40° C., the mixture was stirred at 600 rpm for about 6 hours, then cooled to 10° C. within 6 hours and kept stirring overnight. The obtained solid was re-equilibrated in 20 mL EtOH/water=1/9 at 50° C. for about 6 hours, then gradually cooled to 10° C. in 6 hours and stirred overnight. The solid was separated by suction filtration and dried at 50° C. under vacuum overnight.

Example 4: About 18 g Compound A amorphous free form was weighed into crystallizer. 200 mL of ACN/water=1/9 (v/v) was added. The mixture was stirred at 150 rpm at 40° C. for about 6 hours. then gradually cooled to room temperature and stirred overnight. The solid was isolated by filtration and subsequently dried at 50° C. under vacuum overnight. About 17.2 g of white solid was obtained.

### 2) Characterization of Compound A 4-Hydroxybenzoate Salt

a. XRPD Pattern of Compound A 4-Hydroxybenzoate Salt

(See FIG. 34 for XRPD pattern, strongest peaks are shown below)

| Index | Angle | d Value | Rel. Intensity | Intensity |
|---|---|---|---|---|
| 1 | 9.28° | 9.52101 Å | 31.8% | |
| 2-A | 10.98° | 8.05431 Å | 100.0% | Strong |
| 3-B | 11.78° | 7.50517 Å | 72.6% | Strong |
| 4 | 15.00° | 5.90230 Å | 25.4% | |
| 5 | 15.71° | 5.63573 Å | 36.6% | |
| 6-C | 16.79° | 5.27514 Å | 50.6% | Medium |
| 7 | 18.57° | 4.77480 Å | 31.7% | |
| 8-D | 20.00° | 4.43664 Å | 67.5% | Medium |
| 9 | 22.02° | 4.03389 Å | 68.1% | |
| 10 | 24.09° | 3.69064 Å | 32.8% | |
| 11 | 24.98° | 3.56219 Å | 32.9% | |

b. DSC Thermogram of Compound A 4-Hydroxybenzoate Salt

(See FIG. 35)

c. TGA Thermogram of Compound A 4-Hydroxybenzoate Salt

(See FIG. 36)

d. Preparation Method for Compound A 4-Hydroxybenzoate Salt

Example 1: About 53 mg of Compound A (amorphous) and 1 equivalent molar mass 4-hydroxybenzoic acid were weighed into a vial, then 0.5 mL ter-Butyl methyl ether was added and mixed at 50° C. for about 2 hours. The samples were then cooled to 25° C. and continued to slurry overnight. The solid was collected and re-suspended in 0.2 mL of tetrahydrofuran, then 0.2 mL of heptane was added and the mixture was slurried at 25° C. for 3 days. The solid was obtained by centrifuge filtration.

Example 2: About 53 mg of Compound A (amorphous) and 1 equivalent molar mass 4-hydroxybenzoic acid were weighed into a vial, then 0.5 mL ethyl acetate/heptane (v/v,1/1) was added and mixed at 50° C. for about 2 hours. The samples were then cooled to 25° C. and continued to slurry overnight. The sample was evaporated to dryness, then 0.2 mL of acetone and 0.5 mL of heptane was added, and the mixture was slurried at 25° C. for 3 days. The solid was obtained by centrifuge filtration.

Example 3: About 212 mg Compound A free form and 1 equivalent molar mass of 4-hydroxybenzoic acid was weighed into a vial. 1 mL of THF was added, and clear solution was obtained. Then 2 mL of heptane was added slowly. Gel-like sample was formed, a little bit of seed was added and slurried overnight. The obtained solid was filtrated and dried at 40° C. for 3 hours under vacuum.

Example 4: About 212 mg Compound A free form and 1 equivalent molar mass of 4-hydroxybenzoic acid was weighed into a vial. 1.2 mL of acetone was added. Clear solution was obtained firstly, then some solid precipitated out after several minutes slurry. The obtained solid was filtrated and dried at 40° C. for 3 hours under vacuum.

Example 5: About 2.12 g Compound A free form and 1 equiv. molar mass of 4-hydroxybenzoic acid was weighed into a vial. 10 mL of acetone was added, and clear solution was obtained firstly, then some solid precipitated out after several minutes slurry. The obtained solid was filtrated and dried at 50° C. overnight under vacuum.

Example 6: About 2.1 g Compound A amorphous free form and 552 mg of 4-hydroxybenzoic acid were weighed and added into crystallizer. Then 15 mL of ethanol was added. Clear solution was obtained at 45° C. And the solution was gradually cooled to 40° C. and seed was added. The mixture was cooled down to 40° C. within 6 hours and stirred overnight. The solids were isolated by filtration and subsequently dried at 50° C. under vacuum for about 4 hours. About 1.2 g of white solid was obtained.

### 3) Characterization of Compound A 3,4-Dihydroxybenzoate Salt

a. XRPD Pattern of Compound A 3,4-Dihydroxybenzoate Salt

(See FIG. 37 for XRPD pattern, strongest peaks are shown below)

| Index | Angle | d Value | Rel. Intensity | Intensity |
|---|---|---|---|---|
| 1-A | 10.48° | 8.43768 Å | 63.7% | Medium |
| 2-B | 11.75° | 7.52656 Å | 100.0% | Strong |
| 3 | 14.62° | 6.05531 Å | 31.3% | |
| 4 | 14.79° | 5.98582 Å | 31.1% | |
| 5 | 15.16° | 5.83971 Å | 32.2% | |
| 6-C | 16.27° | 5.44296 Å | 37.3% | Medium |
| 7 | 19.04° | 4.65631 Å | 44.5% | |
| 8-D | 19.70° | 4.50180 Å | 94.7% | Strong |
| 9 | 23.41° | 3.79688 Å | 30.8% | |
| 10 | 24.18° | 3.67776 Å | 59.4% | |

b. DSC Thermogram of Compound A 3,4-Dihydroxybenzoate Salt

(See FIG. 38)

c. TGA Thermogram of Compound A 3,4-Dihydroxybenzoate Salt

(See FIG. 39)

d. Preparation Method for Compound A 3,4-Dihydroxybenzoate Salt

Example 1: About 53 mg of Compound A (amorphous) and 1 equivalent molar mass 3,4-dihydroxybenzoic acid were weighed into a vial, then 0.5 mL acetone was added and mixed at 50° C. for about 2 hours. The samples were then cooled to 25° C. and continued to slurry overnight. The solid was obtained by centrifuge filtration.

Example 2: About 53 mg of Compound A (amorphous) and 1 equivalent molar mass 3,4-dihydroxybenzoic acid were weighed into a vial, then 0.5 mL acetonitrile was added and mixed at 50° C. for about 2 hours. The samples were then cooled to 25° C. and continued to slurry overnight. The solid was obtained by centrifuge filtration.

Example 3: About 1 g Compound A free form and 1 equiv. molar mass of 3,4-dihydroxybenzoic acid was weighed into 20 ml vial. Then 8 mL of acetone was added and mixed with 450 rpm at 50° C. for nearly 1 hour. Clear solution was obtained at first. Some seed was added into the solution, and some solid precipitate out after 1 hour equilibration. Then the sample was cooled to 25° C. and continue to slurry overnight. About 1 g of white solid was obtained.

### Compound A Stability Data

| | Physical Forms | | | | | |
|---|---|---|---|---|---|---|
| | Free form | | Free form | | 4-hydroxy | |
| | Modification A | | amorphous | | benzoate | |
| | Batch or sample ID | | | | | |
| | Compound A - | | Compound A - | | Compound A -) | |
| | Initial purity (%) | | | | | |
| | 99.3 | white | 99.3 | white | 99.9 | white |
| Test Conditions | DP[%] | CL | DP[%] | CL | DP[%] | CL |

Solid state, 2 weeks 80° C., 11% RH

| | | | | | | |
|---|---|---|---|---|---|---|
| Bulk (HPLC) | 1.4 | A | 5.5 | A | 0.2 | A |

| | | | |
|---|---|---|---|
| Bulk (XRPD) | No change | No change | No change |

Solid state, 2 weeks 80° C./75% RH

| | | | | | | |
|---|---|---|---|---|---|---|
| Bulk (HPLC) | 12.6 | gel | 15.3 | gel | 1.5 | A |

| | | | |
|---|---|---|---|
| Bulk (XRPD) | gel | gel | No change |

Solid state, 2 weeks 50° C./75% RH

| | | | | | | |
|---|---|---|---|---|---|---|
| Bulk (HPLC) | 1.3 | A | 2.1 | A | 0.2 | A |

| | | | |
|---|---|---|---|
| Bulk (XRPD) | No change | No change | No change |

Xenon light (1200 kLuxh, 25° C.)

| Bulk clear vial (HPLC) | 4.1 | B | 7.4 | B | 0.2 | A |
|---|---|---|---|---|---|---|
| Bulk clear vial (XPRD) | No change | | No change | | No change | |
| Bulk amber vial (HPLC) | 1.0 | A | 1.0 | A | 0.2 | A |

| | | | |
|---|---|---|---|
| Bulk amber vial (XRPD) | No change | No change | No change |

Excipient compatibility, 2 weeks 70° C./11% RH

| | | | | | | |
|---|---|---|---|---|---|---|
| 1% in mixture 1 | 1.3 | A | — | — | 0.4 | A |
| 1% in mixture 2 | 1.2 | A | — | — | 0.1 | A |
| 1% in mixture 3 | 1.1 | A | — | — | 0.2 | A |
| HGC capsule powder | 1.2 | A | — | — | 0.3 | A |
| HPMC capsule powder | 1.5 | A | — | — | 0.6 | A |

Excipient compatibility, 2 weeks 70° C./75% RH

| | | | | | | |
|---|---|---|---|---|---|---|
| 1% in mixture 1 | 6.1 | gel | — | — | 3.6 | A |
| 1% in mixture 2 | 7.8 | gel | — | — | 2.3 | A |
| 1% in mixture 3 | 11.8 | gel | — | — | 1.5 | A |
| HPC capsule powder | 3.3 | gel | — | — | 1.3 | A |
| HPMC capsule powder | 6.6 | gel | — | — | 3.1 | A |

Excipient compatibility, 2 weeks 50° C./75% RH

| | | | | | | |
|---|---|---|---|---|---|---|
| 1% in mixture 1 | 1.4 | A | — | — | 0.3 | A |
| 1% in mixture 2 | 2.5 | A | — | — | 0.2 | A |
| 1% in mixture 3 | 1.4 | A | — | — | 0.2 | A |
| HGC capsule powder | 1.2 | A | — | — | 0.3 | A |
| HPMC capsule powder | 1.7 | A | — | — | 0.6 | A |

Degradation products (DP) and Color (CL)
↓ Suspension
* Clear solution after stress test
— Test not performed
A No change
B Slight discoloration
C Medium discoloration
D Strong discoloration
DPs are analyzed by HPLC. They are calculated as area-% products or against external standards.
HPMC = Hydroxypropyl Methylcellulose
HGC = Hard gelatin capsule

### Compound A Solubility Data

| Physical Forms | | |
|---|---|---|
| Free form | Free form | 4- |
| Modification A | amorphous | hydroxybenzoate |
| Batch or sample ID | | |
| Compound A - | Compound A - | Compound A |

Solubility (in mg/mL after equilibration at 25° C. for 24 h, target concentration 20 mg/10 mL, final
pH and XRPD of solid residues, use 0.45 μm PVDF membrane for separation if needed)

| | Sol. | | Sol. | | Sol. | |
|---|---|---|---|---|---|---|
| Parameter | (pH) | XRPD | (pH) | XRPD | (pH) | XRPD |
| pH 1.2, 100 mM HCl | >2, | // | >2 | // | >2, | // |
| | (1.16) | | (1.19) | | (1.18) | |
| pH 3.0, 50 mM | >2, | // | >2, | // | >2, | // |
| citrate buffer | (3.10) | | (3.13) | | (3.12) | |
| pH 4.7, 50 mM | >2, | // | >2, | // | >2, | // |
| acetate buffer | (4.86) | | (4.89) | | (4.78) | |
| pH 6.8, 50 mM | >2, | // | >2, | // | >2, | // |
| phosphate buffer | (6.95) | | (6.97) | | (6.75) | |
| pH 6.8, 50 mM | >2, | // | >2, | // | >2, | // |
| phosphate buffer | (6.91) | | (6.87) | | (6.72) | |
| with 50 mM Na | | | | | | |
| taurocholate | | | | | | |
| SGF pH 2.0 | >2, | // | >2, | // | >2, | // |
| | (2.39) | | (2.37) | | (2.36) | |
| FaSSIF V2 | ~2, | // | >2, | // | >2, | // |
| pH 6.5 | (8.07) | | (7.98) | | (6.55) | |
| FeSSIF V2 | >2, | // | >2 | // | >2, | // |
| pH 5.8 | (5.98) | | (5.93) | | (5.86) | |
| Water | 0.27, | No | 0.28, | No | >2, | // |
| | (9.12) | change | (9.26) | change | (7.66) | |

Explanation
“—”: no carried out
“//”: not carried out because substance too soluble in the medium

### Properties of Compound A

| | Physical form of Compound A | | |
|---|---|---|---|
| | Free form | Free form | 4- |
| Parameter | Modification A | amorphous | hydroxybenzoate |
| Thermal properties | | | |
| XRPD (crystallinity) | High | Amorphous | High |
| After heating and | | | |
| cooling- | | | |
| DSC | Tg = 95.6° C. | — | Melt/ |
| | | | decomposition |
| Hygroscopicity | | | |
| Loss on drying by TGA | 0.4% (110° C.) | 1.1% (119° C.) | 0.5% (200° C.) |
| [%] | | | |
| After 1 day at 92% RH | | | |
| Loss on drying by TGA | 1.0% (110° C.) | 2.5% (119° C.) | 0.7% (200° C.) |
| [%] | | | |
| XRPD | No change | No change | No change |
| After 1 day at 80% RH | | | |
| Loss on drying by TGA | 0.7% (110° C.) | 2.0% (119° C.) | 0.6% (200° C.) |
| [%] | | | |
| XRPD | No change | No change | No change |

—Test not performed

### Brief Summary of the Selection of Free Form

The free form (also known as “Modification A”) was chosen for development because it shows excellent properties in the aspects of crystallinity, solubility, stability and polymorphic behavior. In addition, because no counter ion is present there is no safety concerns relating to the choice of counter ion.

The 4-hydroxybenzoate salt also had excellent properties in the aspects of crystallinity, solubility, stability and polymorphic behavior. However, it was not chosen for development because there is insufficient safety data relating to the counter ion.

1. A crystalline form of 2-((2S,3S,4S)-5-Chloro-6-fluoro-3-methyl-2-((methylamino)methyl)-2-phenyl-2,3-dihydrobenzofuran-4-yl)-3-fluoro-4-methoxybenzamide (Compound B) or pharmaceutically acceptable solvate and/or salt thereof.

2. A crystalline form of 4-((2S,4S)-5-Chloro-6-fluoro-2-phenyl-2-((S)-pyrrolidin-2-yl)-2,3-dihydrobenzofuran-4-yl)-5-fluoro-6-(2-hydroxyethoxy)-N-methylnicotinamide (Compound A) or pharmaceutically acceptable solvate and/or salt thereof.

3. The crystalline form according to claim 1, wherein the Compound B is in the form of a succinate salt.

4. The crystalline form according to claim 3, characterized by an X-ray powder diffraction pattern comprising peaks at four or more 2θ values (e.g. 5 or more, e.g. 6 or more, e.g. 7 or more, e.g. 8 or more, e.g. 9 or more, e.g. 10 or more, e.g. 11 or more, e.g. 12 or more, e.g. all 13 2θ values) selected from the group consisting of:
Angle (2θ)
11.93° ± 0.2°
13.26° ± 0.2°
13.77° ± 0.2°
15.12° ± 0.2°
16.99° ± 0.2°
17.39° ± 0.2°
19.92° ± 0.2°
20.73° ± 0.2°
21.06° ± 0.2°
23.45° ± 0.2°
24.11° ± 0.2°
26.37° ± 0.2°
26.66° ± 0.2°
wherein the temperature is about room temperature and the radiation used has a wavelength of 1.54060 Å, and preferably wherein the X-ray powder diffraction pattern comprises at least the peaks at 13.26°±0.2°, 16.99°±0.2°, 19.92°±0.2° and 26.66°±0.2°.

5-7. (canceled)

8. The crystalline form according to claim 1, wherein the Compound B is in the form of a malate salt.

9-12. (canceled)

13. The crystalline form according to claim 1, wherein the Compound B is in the form of a lactate salt.

14-17. (canceled)

18. The crystalline form according to claim 1, wherein the Compound B is in the form of a benzoate salt.

19-22. (canceled)

23. The crystalline form according to claim 1, wherein the Compound B is in the form of a glutamate salt.

24-27. (canceled)

28. The crystalline form according to claim 1, wherein the Compound B is in the form of a maleate salt.

29-32. (canceled)

33. The crystalline form according to claim 1, wherein the Compound B is in the form of a malonate salt.

34-41. (canceled)

42. The crystalline form according to claim 1, wherein the Compound B is in the form of a mesylate salt.

43-46. (canceled)

47. The crystalline form according to claim 1, wherein the Compound B is in free form.

48. The crystalline form according to claim 47, wherein the Compound B is in the form of Compound B free form 2-methyl-2-butanol solvate

49-52. (canceled)

53. The crystalline form according to claim 2, wherein the Compound A is free form Compound A or a solvate thereof.

54. The crystalline form according to claim 53, characterized by an X-ray powder diffraction pattern comprising four or more 2θ values (e.g. 5 or more, e.g. 6 or more, e.g. 7 or more, e.g. 8 or more, e.g. 9 or more, e.g. all ten 2θ values) selected from the group consisting of:
```
Angle
  7.00° ± 0.2°
  9.21° ± 0.2°
10.98° ± 0.2°
16.06° ± 0.2°
17.24° ± 0.2°
17.82° ± 0.2°
19.25° ± 0.2°
21.80° ± 0.2°
22.84° ± 0.2°
24.69° ± 0.2°
```
wherein the temperature is about room temperature and the radiation used has a wavelength of 1.54060 Å, and preferably wherein the X-ray powder diffraction pattern comprises at least the peaks at 7.00°±0.2°, 9.21°±0.2°, 10.98°±0.2° and 21.80°±0.2°.

55-58. (canceled)

59. The crystalline form according to claim 2, wherein the Compound A is in the form of a 4-hydroxybenzoate salt or a 3,4-dihydroxybenzoate salt.

60. The crystalline form according to claim 59, characterized by an X-ray powder diffraction pattern comprising four or more 2θ values (e.g. 5 or more, e.g. 6 or more, e.g. 7 or more, e.g. 8 or more, e.g. 9 or more, e.g. 10 or more, e.g. all 11 2θ values) selected from the group consisting of:
Angle
9.28° ± 0.2°
10.98° ± 0.2°
11.78° ± 0.2°
15.00° ± 0.2°
15.71° ± 0.2°
16.79° ± 0.2°
18.57° ± 0.2°
20.00° ± 0.2°
22.02° ± 0.2°
24.09° ± 0.2°
24.98° ± 0.2°
wherein the temperature is about room temperature and the radiation used has a wavelength of 1.54060 Å, and preferably wherein the X-ray powder diffraction pattern comprises at least the peaks at 10.98°±0.2°, 11.78°+0.2°, 16.79°±0.2° and 20.00°±0.2°.

61-68. (canceled)

69. A pharmaceutical composition comprising the crystalline form of claim 1 and a pharmaceutically acceptable carrier.

70-72. (canceled)

73. A method of treating a disease or condition mediated by YAP overexpression and/or YAP amplification and/or YAP/TAZ-TEAD interaction, or a method of treating a cancer or tumor harboring (i) one or more YAP/TAZ fusions; (ii) one or more NF2/LATS1/LATS2 truncating mutations or deletions; or (iii) one or more functional YAP/TAZ fusions; said method comprising administering to a subject in need thereof, a therapeutically effective amount of a crystalline form according to claim 1.

74. (canceled)

75. The method according to claim 73, wherein the cancer, tumor disease or condition is selected from mesothelioma (including pleural mesothelioma, malignant pleural mesothelioma, peritoneal mesothelioma, pericardial mesothelioma and mesothelioma of the tunica vaginalis), carcinoma (including cervical squamous cell carcinoma, endometrial carcinoma, esophageal squamous cell carcinoma, esophageal adenocarcinoma, urothelial carcinoma of the bladder and squamous cell carcinoma of the skin), poroma (benign poroma), porocarcinoma (including malignant porocarcinoma), supratentorial ependymoma (including childhood supratentorial ependymoma), epithelioid hemangioendothelioma (EHE), ependymal tumor, a solid tumor, breast cancer (including triple negative breast cancer), lung cancer (including non-small cell lung cancer), ovarian cancer, colorectal cancer (including colorectal carcinoma), melanoma, pancreatic cancer (including pancreatic adenocarcinoma), prostate cancer, gastric cancer, esophageal cancer, liver cancer (including hepatocellular carcinoma, cholangiocarcinoma and hepatoblastoma), neuroblastoma, Schwannoma, kidney cancer, sarcoma (including rhabdomyosarcoma, embryonic rhabdomyosarcoma (ERMS), osteosarcoma, undifferentiated pleomorphic sarcomas (UPS), Kaposi's sarcoma, soft-tissue sarcoma and rare soft-tissue sarcoma), bone cancer, brain cancer, medulloblastoma, glioma, meningioma, and head and neck cancer (including head and neck squamous cell carcinoma).
